(19) United States
(12) Patent Application Publication
(10) Pub. No.: US 2015/0150960 A1
(43) Pub. Date: Jun. 4, 2015

(54) PROTECTION AGAINST DENGUE VIRUS AND PREVENTION OF SEVERE DENGUE DISEASE

(71) Applicant: Sujan Shresta, San Diego, CA (US)

(72) Inventor: Sujan Shresta, San Diego, CA (US)

(73) Assignee: LA JOLLA INSTITUTE FOR ALLERGY AND IMMUNOLOGY, San Diego, CA (US)

(21) Appl. No.: 14/128,268

(22) PCT Filed: Jun. 25, 2012
(86) PCT No.: PCT/US2012/044071

§ 371 Date: Apr. 7, 2014

Related U.S. Application Data
(60) Provisional application No. 61/391,882, filed on Oct. 11, 2010.
(60) Provisional application No. 61/358,142, filed on Jun. 24, 2010.

(51) Int. Cl. A61K 39/12; C12N 7/00

(52) U.S. Cl. CPC A61K 39/12; C12N 7/00; C12N 2770/24134; A61K 2039/5252

(57) ABSTRACT

The invention provides uses, methods and compositions for eliciting, stimulating, inducing, promoting, increasing, or enhancing an anti-Dengue virus T cell response in a subject.

### RELATED APPLICATION INFORMATION

This application is a U.S. National Phase of International Application No. PCT/US2012/044071, filed Jun. 25, 2012, which designated the U.S. and that International Application was published under PCT Article 21(2) in English, which is a continuation-in-part of application serial no. PCT/US2011/041889, filed Jun. 24, 2011, and claims priority to U.S. Provisional Application No. 61/391,882, filed Oct. 11, 2010 and U.S. Provisional Application No. 61/358,142, filed Jun. 24, 2010, all of which applications are incorporated herein by reference in their entirety.

### GOVERNMENT SUPPORT

This invention received government support from the National Institutes Health grants AI060989, AI077099, U54 AI057157, U01A082185 and National Institutes of Health Contract HHSN272200900042C. The government has certain rights in the invention.

### FIELD OF INVENTION

The invention relates to Dengue virus proteins, subsequences and portions thereof, including DENV epitopes and modifcations of DENV proteins, subsequences and portions thereof, and uses and methods for eliciting, stimulating, inducing, promoting, increasing, or enhancing an anti-Dengue virus T cell response in a subject without sensitizing the subject to severe dengue disease upon subsequent Dengue virus infection.

### INTRODUCTION

Dengue virus (DENV, or DV) is a mosquito-borne RNA virus in the Flaviviridae family, which also includes West Nile Virus (WNV), Yellow Fever Virus (YFV), and Japanese Encephalitis Virus (JEV). The four serotypes of DENV (DENV1-4) share approximately 65-75% homology at the amino acid level (Fu, et al. Virology 188:953 (1992)). Infections with DENV can be asymptomatic, or cause disease ranging from dengue fever (DF) to dengue hemorrhagic fever (DHF) and dengue shock syndrome (DSS) (WHO, Dengue: Guidelines for diagnosis, treatment, prevention and control (2009)). DF is a self-limiting illness with symptoms that include fever, headache, myalgia, retro-orbital pain, nausea, and vomiting. DHF and DSS are characterized by increased vascular permeability, thrombocytopenia, hemorrhagic manifestations, and in the case of DSS, shock, which can be fatal. The incidence of DENV infections has increased 30-fold in the past 50 years (WHO, Dengue: Guidelines for diagnosis, treatment, prevention and control (2009)). DF and DHF DSS are a significant cause of morbidity and mortality worldwide, and therefore a DENV vaccine is a global public health priority. However, vaccine development has been challenging, as a vaccine should protect against all four DENV serotypes (Whitehead, et al. Nat Rev Microbiol 5:518 (2007)).

Severe dengue disease (DHF/DSS) most often occurs in individuals experiencing a secondary infection with a heterologous DENV serotype, suggesting the immune response contributes to the pathogenesis (Sangkawibha, et al. Am J Epidemiol 120:653 (1984); Guzman, et al. Am J Epidemiol 152:793 (1997)). To explain the occurrence of DHF/DSS with secondary infection, two dominant hypotheses: (i) antibody (Ab) dependent enhancement of infection (ADE) and (ii) original T cell antigenic sin have been postulated. Under the ADE hypothesis, serotype cross-reactive antibodies enhance infection of FcγR⁺ cells during a secondary infection resulting in higher viral loads and more severe disease via a phenomenon known as antibody-dependent enhancement (ADE) (Morens, et al. Clin Infect Dis 19:500 (1994); Halstead, Adv Virus Res 60:421 (2003)). Recent studies have demonstrated DENV-specific antibody can enhance disease in mice (Zellweger, et al. Cell Host Microbe 7: 128 (2010); Balsitis, et al. PLoS Pathog 6:e1000790 (2010)). Under the original T cell antigenic sin hypothesis, it is proposed that serotype cross-reactive memory T cells may respond sub-optimally during secondary infection and contribute to the pathogenesis (Mathew, et al. Immunol Rev 225:300 (2008)). Accordingly, studies have shown serotype cross-reactive T cells can exhibit an altered phenotype in terms of cytokine production and degranulation (Mangada, et al. J Immunol 175:2676 (2005); Mongkolsapaya, et al. Nat Med 9:921 (2003); Mongkolsapaya, et al. J Immunol 176:3821 (2006)). However, another study found the breadth and magnitude of the T cell response during secondary DENV infection was not significantly associated with disease severity (Simmons, et al. J Virol 79:5665 (2005)).

CD4⁺ T cells can contribute to the host response to pathogens in a variety of ways. They produce cytokines and can mediate cytotoxicity. They also help B cell responses by inducing immunoglobulin class switch recombination (CSR), and help prime the CD8⁺ T cell response. CD4⁺ T cells can help the CD8⁺ T cell response indirectly by activating APCs, for example via CD40L/CD40 (Bevan, Nat Rev Immunol 4:595 (2004)). CD40L on CD4⁺ T cells is important in activating B cells as well (Elgueta, et al. Immunol Rev 229:152 (2009)). CD4⁺ T cells can also induce chemokine production that attracts CD8⁺ T cells to sites of infection (Nakanishi, et al. Nature 462:510 (2009)). However, the requirement for CD4⁺ T cell help for antibody and CD8⁺ T cell responses is not absolute, and may be specific to the pathogen and/or experimental system. For instance, it has been shown that CSR can occur in the absence of CD4+ T cells (Stavnezer, et al. Annu Rev Immunol 26:261 (2008)), and the primary CD8⁺ T cell response is CD4-independent under inflammatory conditions (Bevan, Nat Rev Immunol 4:595 (2004)). This suspected dual role of T cells in protection and pathogenesis is difficult to study in humans, since in most donor cohorts the time point and in case of secondary infections the sequence of infection is unknown, and does not allow direct correlations with T cell responses.

Although many studies have investigated the role of T cells in DENV pathogenesis, the role of T cells in protection versus pathogenesis during DENV infections was, prior to the disclosure herein,unknown. In this regard, the lack of an adequate animal model made such studies impossible, as mice are resistant to infection with this human pathogen (Yauch, et al. Antiviral Res 80:87 (2008)). A mouse model, which allows investigation of adaptive immune responses restricted by human histocompatibility complex (MHC) molecules to DENV infection, would shed light on the role of T cells in protection and/or pathogenesis.

Mice transgenic for human leukocyte antigens (HLA) are widely used to study T cell responses restricted by human MHC molecules and studies in other viral systems have shown the valuable impact of HLA transgenic mice in epitope identification (Kotturi, et al. Immunome Res 6:4 (2010); Kotturi, et al. Immunome Res 5:3 (2009); Pasquetto, et al. J Immunol 175:5504 (2005)). Recently, a mouse-passaged DENV2 strain, S221, which does not replicate to detectable levels in wild-type C57BL/6 mice, was reported to replicate in IFN-α/R^{‘}′^{″} mice (Yauch, et al. J Immunol 182:4865 (2009)). Using S221 and IFN-αβR^{-/-} mice, a protective role for CD8⁺ T cells in the response to primary DENV2 infection was reported (Yauch, et al. J Immunol 182:4865 (2009)). The DENV field has been focusing vaccine development efforts towards induction of humoral immunity, because as with other viral vaccines, DENV-specific antibodies (Abs) are assumed to provide the key means of protection against natural infection. However, epidemiologic studies have shown that severe dengue disease is preferentially associated with secondary infections in humans and infants born to DENV-immune mothers. Moreover, recent studies using mouse models have shown DENV-specific Abs can contribute to pathogenesis by mediating antibody-dependent enhancement of infection (ADE). ADE has been demonstrated to enhance viremia and severity of dengue disease in non-human primate (Goncalvez, et al. Proc Natl Acad Sci U S A 104:9422-9427 (2007); Halstead J Infect Dis 140:527-533 (1979); Halstead, et al. J Infect Dis 128:15-22 (1973)) and mouse (Balsitis, et al. PLoS Pathog 6:e1000790 (2010); Zellweger, et al. Cell Host Microbe 7:128-139 (2010)) models, respectively. Despite the potential for ADE, based on a vast number of publications on antibody-mediated protection (reviewed in Innis CAB International, Wallingford, Oxon, UK; New York (1997); Murphy, et al. Annual Rev. of Immunol. 29:587-619 (2011)), the consensus in the field is that induction of protective levels of neutralizing Abs should be the primary objective of dengue vaccination.

Direct evidence linking T cells to increased viremia or pathology has never been shown, although numerous studies have examined T cell responses in the context of Dengue virus (DENV) pathogenesis. Although limited, studies examining T cell-mediated protection against DENV (Calvert, et al. Journal General Virol. 87:339-346 (2006); Kyle, et al. Virology 380:296-303 (2008)) generally assume that T cells play at most a secondary role in protection against DENV reinfection.

### SUMMARY

The invention is based, in part, on the discovery that DENV vaccine-induced antibody response can mediate ADE and enhance (worsen) DENV disease severity. The invention is also based, in part, on the discovery that CD8+ T cell responses dictate the extent of dengue vaccine-mediated protection. The invention is further based, in part, on the discovery that CD8+ T cell responses can provide protection against DENV infection, including protection against heterologous DENV serotypes, even in the presence of enhancing antibodies.

Thus, the invention provides uses, methods and compositions for eliciting, stimulating, inducing, promoting, increasing, or enhancing an anti-Dengue virus T cell response in a subject. In one embodiment, a use or method includes administering to the subject an amount of a Dengue virus protein or subsequence thereof sufficient to elicit an anti-Dengue virus T cell response in the subject. In particular aspects, a use or method elicits, stimulates, induces, promotes, increases, or enhances an anti-Dengue virus T cell response in a subject without sensitizing the subject to severe dengue disease (e.g., ADE) upon a secondary or subsequent Dengue virus exposure or infection.

In another embodiment, a use or a method of vaccinating a subject against or providing a subject with protection against a Dengue virus infection includes administering to the subject an amount of a Dengue virus protein or subsequence thereof sufficient to vaccinate the subject against or protect the subject against the Dengue virus infection. In a particular, aspect, the use or method does not sensitize the subject to severe dengue disease upon a secondary or subsequent Dengue virus exposure or infection.

In a further embodiment, a use or method of treating a subject for a Dengue virus infection includes administering to the subject an amount of a Dengue virus protein or subsequence thereof sufficient to treat the subject for the Dengue virus infection. In a particular, aspect, the use or method does not sensitize the subject to severe dengue disease upon a secondary or subsequent Dengue virus exposure or infection.

The invention also provides compositions including an amount of a Dengue virus protein or subsequence or portion or modification thereof. In various embodiments, these compositions are for use in: eliciting, stimulating, inducing, promoting, increasing, or enhancing an anti-Dengue virus T cell response in a subject, optionally without elicting or sensitizing the subject to severe dengue disease upon a secondary or subsequent Dengue virus infection or exposure; in providing a subject with protection against a Dengue virus infection or pathology, or one or more physiological disorders, illness, diseases or symptoms caused by or associated with Dengue virus infection or pathology, optionally without elicting or sensitizing the subject to severe dengue disease upon a secondary or subsequent Dengue virus infection; in vaccinating a subject against a Dengue virus infection without elicting or sensitizing the subject to severe dengue disease upon a secondary or subsequent Dengue virus infection or exposure; and in treating a subject for a Dengue virus infection, optionally without elicting or sensitizing the subject to severe dengue disease upon a secondary or subsequent Dengue virus infection or exposure.

In additional particular embodiments, the uses, methods and compositions are useful for eliciting, stimulating, inducing, promoting, increasing, or enhancing an anti-Dengue virus CD8⁺ T cell response, optionally without elicting or sensitizing the subject to severe dengue disease upon a secondary or subsequent Dengue virus infection or exposure. In certain embodiments , anti-Dengue virus CD8+ T cell response is directed and/or protective against a plurality of different Dengue virus serotypes. In particular embodiments, the anti-Dengue virus CD8+ T cell response is directed and/or protective against at least two Dengue virus serotypes selected from DENV1, DENV2, DENV3 and DENV4.

In different embodiments of the uses, methods and compositions, the protein comprises or consists of a Dengue virus serotype 1, 2, 3 or 4 protein.

In certain embodiments, a Dengue virus protein is a non-structural protein such as, for example, NS1, NS2A, NS2B, NS3, NS4A, NS4B or NS5. In other embodiments, a Dengue virus protein is a structural protein such as, for example, Dengue virus envelope (E) protein, membrane (M) protein or core protein.

In uses, methods and compositions of the invention include those that do not substantially sensitize a subject to severe dengue disease (e.g., via ADE), or elicit, induce, stimulate or promote severe dengue disease, upon a secondary or subsequent Dengue virus infection or exposure. In certain embodiments, severe dengue disease is mediated by antibody dependent enhancement (ADE). In certain embodiment, the severe Dengue virus disease comprises antibody-dependent enhancement of infection.

In certain embodiments of the uses, methods and compositions, the protein administered consists of a single Dengue virus serotype. In other embodiments of the uses, methods and compositions, protein administered comprises a plurality of single Dengue virus serotype proteins administered. In still further embodiments of the uses, methods and compositions, protein administered comprises or consists of one or more Dengue virus serotype 1, 2, 3 or 4 proteins. In particular different embodiments of the uses, methods and compositions, protein administered comprises or consists of one or more Dengue virus serotype 1 proteins, and not a Dengue virus serotype 2, 3 or 4 protein; protein administered comprises or consists of one or more Dengue virus serotype 2 proteins, and not a Dengue virus serotype 1, 3 or 4 protein; protein administered comprises or consists of one or more Dengue virus serotype 3 proteins, and not a Dengue virus serotype 1, 2 or 4 protein; or protein administered comprises or consists of one or more Dengue virus serotype 4 proteins, and not a Dengue virus serotype 1, 2 or 3 protein.

In certain embodiments of the uses, methods and compositions, administration of a protein of a first Dengue virus serotype is effective to vaccinate or provide the subject with protection against one or more Dengue virus serotypes distinct from the first Dengue virus serotype. In particular different embodiments of the uses, methods and compositions, administration of a Dengue virus serotype 1 protein is effective to vaccinate or provide the subject with protection against one or more of Dengue virus serotypes 2, 3 or 4; administration of a Dengue virus serotype 2 protein is effective to vaccinate or provide the subject with protection against one or more of Dengue virus serotypes 1, 3 or 4; administration of a Dengue virus serotype 3 protein is effective to vaccinate or provide the subject with protection against one or more of Dengue virus serotypes 1, 2 or 4; or administration of a Dengue virus serotype 4 protein is effective to vaccinate or provide the subject with protection against one or more of Dengue virus serotypes 1, 2 or 3.

In other embodiments of the uses, methods and compositions, administration of a protein of a first Dengue virus serotype is effective to treat the subject for infection with one or more Dengue virus serotypes distinct from the first Dengue virus serotype. In particular different embodiments of the uses, methods and compositions, administration of a Dengue virus serotype 1 protein is effective to treat the subject for infection with one or more of Dengue virus serotypes 2, 3 or 4; administration of a Dengue virus serotype 2 protein is effective to treat the subject for infection with one or more of Dengue virus serotypes 1, 3 or 4; administration of a Dengue virus serotype 3 protein is effective to treat the subject for infection with one or more of Dengue virus serotypes 1, 2 or 4; or administration of a Dengue virus serotype 4 protein is effective to treat the subject for infection with one or more of Dengue virus serotypes 1, 2 or 3.

In certain embodiments, uses, methods and compositions reduce Dengue virus titer, increasing or stimulating Dengue virus clearance, reduce or inhibit Dengue virus proliferation, reduce or inhibit increases in Dengue virus titer or Dengue virus proliferation, reduce the amount of a Dengue virus protein or the amount of a Dengue virus nucleic acids, or reduce or inhibit synthesis of a Dengue virus protein or a Dengue virus nucleic acid. In other particular embodiments, uses, methods and compositions prevent, reduce, improve or inhibit one or more adverse physiological conditions, disorders, illnesses, diseases, symptoms or complications caused by or associated with Dengue virus infection or pathology. In still further particular embodiments, uses, methods and compositions reduce or inhibit susceptibility to Dengue virus infection or pathology or protect a subject from adverse physiological conditions, disorders, illnesses, diseases, symptoms or complications caused by or associated with an antibody response to a Dengue virus infection.

In other embodiments, invention uses, methods and compositions may be performed or administered prior to exposure to or infection of the subject with the Dengue virus, or substantially contemporaneously with exposure to or infection of the subject with the Dengue virus, or following exposure to or infection of the subject with the Dengue virus. Such exposure or infection includes secondary or subsequent DENV infections (e.g., reinfection).

In further embodiments, invention uses and methods include administering a Dengue virus protein or subsequence or portion or modification thereof in combination with a T-cell stimulatory molecule. In still further embodiments, a composition includes a combination of a Dengue virus protein or portion or modification thereof and a T-cell stimulatory molecule. In particular aspects a T-cell stimulatory molecule is OX40 or CD27.

In particular embodiments of the uses, methods and compositions, the subject is a mammal, for example, a human.

In certain embodiments of the uses, methods and compositions, a subject has not previously been infected with Dengue virus. In other embodiments of the uses, methods and compositions, a subject, prior to administration of the Dengue virus protein, produces antibodies against one or more Dengue virus serotypes. In still further embodiments of the uses, methods and compositions, a subject has previously been infected with Dengue virus.

As disclosed herein, candidate MHC class II (I-A^{b})-binding peptides from the entire proteome of DENV2, which is approximately 3390 amino acids and encodes three structural (core (C), envelope (E), and membrane (M)), and seven non-structural (NS) (NS1, NS2A, NS2B, NS3, NS4A, NS4B, NS5) proteins, were identified. Numerous CD4⁺ T cell and CD8+ T cell epitopes from the structural and non-structural (NS) proteins are also disclosed herein (e.g., Tables 1-4). Immunization with T cell epitopes, such as CD8⁺ or CD4⁺ T cell epitopes, before DENV infection resulted in significantly lower viral loads. While CD4⁺ T cells do not appear to be required for controlling primary DENV infection, immunization contributes to viral clearance.

By way of example, 42 epitopes derived from 9 of the 10 DENV proteins were identified. 80% of the epitopes identified were able to elicit a T cell response in human donors, previously exposed to DENV. The mouse model described herein also reflected response patterns observed in humans. These findings indicate that inducing anti-DENV CD4+ T and/or CD8+ T cell responses by immunization/vaccination will be an effective prophylactic or therapeutic treatment for DENV infection and/or pathology.

In accordance with the invention, there are provided DENV proteins, methods and uses, in which the proteins include or consist of a subsequence, portion, or an amino acid modification of Dengue virus (DV) structural or non-structural (NS) polypeptide sequence from any of DENV serotypes 1, 2, 3 or 4, and the protein elicits, stimulates, induces, promotes, increases, or enhances an anti-DV CD8⁺ T cell response or an anti-DV CD4⁺ T cell response. In one embodiment, a protein includes or consists of a subsequence, portion, or an amino acid modification of Dengue virus (DV) structural core (C), membrane (M) or envelope (E) polypeptide sequence, for example, based upon or derived from a DENV1, DENV2, DENV3 or DENV4 serotype. In another embodiment, a protein includes or consists of a subsequence, portion, or an amino acid modification of Dengue virus (DV) NS1, NS2A, NS2B, NS3, NS4A, NS4B or NS5 polypeptide sequence, for example, based upon or derived from a DENV1, DENV2, DENV3 or DENV4 serotype.

In particular aspects, a protein includes or consists of a structural or non-structural (NS) polypeptide sequence from a DENV serotype 1, 2, 3 or 4. In additional particular aspects, a protein includes or consists of a sequence set forth in Tables 1-4, or a subsequence thereof or a modification thereof. Exemplary modifications include 1, 2, 3, 4, 5 or 6, 7, 8, 9, 10 or more conservative, non-conservative, or conservative and non-conservative amino acid substitutions.

In certain embodiments, a protein, subsequence, portion, or a modification thereof elicits an anti-DV response. In particular aspects, an anti-DV response includes a CD8⁺ T cell response and/or a CD4⁺ T cell response. Such responses can be ascertained, for example, by increased IFN-gamma, TNF-alpha, IL-1alpha, IL-6 or IL-8 production by CD8⁺ T cells in the presence of the protein; and/or increased CD4⁺ T cell production of IFN-gamma, TNF, IL-2, or CD40L in the presence of the protein, or killing of peptide-pulsed target cells.

The invention also provides compositions including the proteins, subsequences, portions, or modifications thereof (e.g., T cell epitopes), such as pharmaceutical compositions. Compositions can include one or more proteins, subsequences, portions, or modifications thereof, such as peptides selected from Tables 1-4, or a subsequence or portion thereof, or a modification thereof, as well as optionally adjuvants.

Proteins, subsequences, portions, and modifications thereof (e.g., T cell epitopes) can be used for stimulating, inducing, promoting, increasing, or enhancing an immune response against Dengue virus (DV) in a subject. In one embodiment, a method includes administering to a subject an amount of a DENV protein, subsequence, portion, or a modification thereof sufficient to stimulate, induce, promote, increase, or enhance an immune response against Dengue virus (DV) in the subject, and/or provide the subject with protection against a Dengue virus (DV) infection or pathology, or one or more physiological conditions, disorders, illness, diseases or symptoms caused by or associated with DV infection or pathology.

DENV proteins, subsequences, portions, and modifications thereof (e.g., T cell epitopes) can also be used for treating a subject for a Dengue virus (DV) infection. In one embodiment, a method includes administering to a subject an amount of a DENV protein, subsequence, portion, or a modification thereof sufficient to treat the subject for the Dengue virus (DV) infection.

Exemplary responses, in vitro, ex vivo or in vivo, elicited by proteins, subsequences, portions, or modifications thereof, such as T cell epitopes include, stimulating, inducing, promoting, increasing, or enhancing an anti-DV CD8⁺ T cell response or an anti-DV CD4⁺ T cell response. In particular aspects, CD8⁺ T cells produce IFN-gamma, TNF-alpha, IL-1alpha, IL-6 or IL-8 in response to T cell epitope, and/or CD4⁺ T cells produce IFN-gamma, TNF, IL-2 or CD40L, or kill peptide-pulsed target cells in response to a T cell epitope. Accordingly, proteins, subsequences, portions, and modifications thereof (e.g., T cell epitopes) can also be used for inducing, increasing, promoting or stimulating anti-Dengue virus (DV) activity of CD8⁺ T cells or CD4+ T cells in a subject.

In various embodiments, multiple proteins, subsequences, portions, or modifications thereof, for example, multiple Dengue virus (DV) proteins, such as T cell epitopes are employed in the methods and uses of the invention. In particular aspects, a Dengue virus (DV) protein, such as a T cell epitope, includes or consists of one or more sequences set forth in Tables 1-4, or a subsequence or portion thereof, or a modification thereof.

### DESCRIPTION OF DRAWINGS

FIG. 1 shows a schematic of an immunization protocol.

FIGS. 2A-2B show levels of viral RNA in the liver of AG129 mice that were immunized with UV-inactivated DENV2 in alum and then challenged with DENV2. A) AG129 mice were immunized s.c. (black circles) or i.p. (black diamonds) with UV-inactivated DENV2 strain S221 (1011 GE) in alum on days −14 and −5, followed by challenge with 5×10⁸ GE of 5221 i.v. on day 0. The control groups represent non-immunized AG129 mice that were treated i.p. with 15 μg of 2H2 (ADE, black squares) or C1.18 (baseline, white squares) 1 hour before viral challenge. B) Serum of AG129 mice immunized as in panel A (200 μl) was transferred i.v. into naïve AG129 mice 1 day before challenge with 5×10⁸ GE of S221 i.v. Levels of viral RNA in the liver were measured 72 hours after infection by qRT-PCR. Each symbol represents an individual animal.

FIGS. 3A-3B show levels of viral RNA in the liver (A) and survival (B) of AG129 mice that were immunized with VRP-DENV2E and then challenged with DENV2. AG129 mice were immunized with VRP-DENV2E (10⁶ GE) via i.f. (IF vaccinated, black circles) or i.p. (IP vaccinated, black triangles) route on days −14 and −5, followed by challenge with 5×10⁸ GE of S221 i.v. on day 0. The control groups represent non-immunized mice that were treated i.p. with 15 μg of 2H2 (ADE, black squares) or C1.18 (baseline, white squares) 1 hour before viral challenge. A) Levels of viral RNA in the liver were measured 72 hours after infection by qRT-PCR. Each symbol represents an individual animal. B) Survival of mice following viral challenge. N =4 mice per group.

FIG. 4 shows levels of viral RNA in the liver of AG129 mice that were immunized with VRP-GFP or VRP-DENV2E and then challenged with DENV2. AG129 mice were immunized i.f. with 10⁶ GE of VRP-GFP (white triangles) or VRP-DENV2E (black triangles) on days −14 and −5, followed by challenge with 5×10⁸ GE of S221 i.v. on day 0. The control groups represent non-immunized AG129 mice that were treated i.p. with 15 μg of 2H2 (ADE, black squares) or C1.18 (baseline, white squares) 1 hour before viral challenge. DENV RNA levels in the liver were measured 72 hours after infection by qRT-PCR. Each symbol represents a mouse.

FIG. 5 shows data indicating that DENV2E provides protection against ADE-DENV challenge. AG129 mice were immunized i.p. with 10⁶ GE of VRP-DENV2 (VRP2) on days −14 and −5, followed by challenge with 5×10⁸ GE of S221 i.v. on day 0 in the presence of isotype control mAb C1.18 (baseline, white circles) or anti-DENV mAb 2H2 (ADE, black circles). Control groups represent non-immunzed AG129 mice that were treated i.p. with 15 μg of 2H2 (ADE, black squares) or C1.18 (baseline, white squares) 1 hour before viral challenge. DENV RNA levels in the liver were measured 72 hours after infection by qRT-PCR. Each symbol represents a mouse.

FIGS. 6A-6B show a comparison of antibody (Ab) responses induced by UV-inactivated DENV2 plus alum versus VRP-DENV2E. AG129 mice were immunized i.p. with 10¹¹ GE of UV-inactivated S221 in alum (diamonds) or DENV2E (triangles) on days −14 and −5, followed by harvest of serum on day −1, as per our standard immunization protocol. A) DENV2-reactive IgG in the sera harvested from the immunized mice was measured by ELISA on plates coated with sucrose gradient purified S221. B) Neutralization activity of the sera used in A was examined by measuring their ability to reduce infection of C6/36 cells by S221.

FIG. 7 shows a schematic of T cell depletion from immunized mice.

FIGS. 8A-8C show the role of T cells in DENV2E vaccine-mediated protection. AG129 mice were immunized i.p. with 10⁶ GE of VRP-DEN2E on days −14 and −5, followed by challenge with 5×10⁸ GE of S221 i.v. on day 0. Separate groups of immunized mice were depleted of CD4+ and/or CD8+ T cells prior to infection, as previously published (Yauch et al., J. Immunol 185:5405 (2010); Yauch et al., J. Immunol. 182:4865 (2009)). Control groups represent non-immunized AG129 mice that were treated i.p. with 15 μg of 2H2 (ADE, black squares) or C1.18 (baseline, white squares) 1 hour before infection. Each symbol represents a mouse. A) DENV RNA levels in the liver of immunized mice that were undepleted (black triangles) or depleted of both CD4+ and CD8+ T cells (white triangles). B) DENV RNA levels in the liver of immunized mice that were undepleted (black triangles) or depleted of either CD4+ (black circles) or CD8+ T cells (black diamonds). For both panels A and B, DENV RNA levels were measured 72 hours after infection by qRT-PCR. C) Serum cytokine levels at 72 hours after infection in the immunized mice that were undepleted (black triangles) or depleted of CD4+ T cells alone (black circles), CD8+ T cells alone (black diamonds), or both CD4+ and CD8+ T cells (white triangles) were measured by multi-plex ELISA.

FIG. 9 shows RNA levels in the liver of AG129 mice adoptively transferred with homologous or heterologous T cells and then challenged with DENV. A129 mice were infected with 10¹⁰ GE of S221 or DENV4 strain H421 (Philippino clinical isolate). 6 weeks later, total T cells from spleens of the DENV-immune mice were isolated by negative selection (Miltenyi MACS system) and transferred i.v. into AG129 mice 1 day before challenge with 5×10⁸ GE of S221 i.v. Liver DENV2 RNA levels on day 3 after infection were measured by qRT-PCR.

FIG. 10 shows viral RNA levels in the liver of CD8+ T cell-sufficient or -depleted AG129 mice with heterologous secondary DENV infection. AG129 mice were infected with 5×10¹⁰ GE of DENV3 strain UNC3001 (Sri Lankan clinical isolate). 21 days later, DENV3-immune mice were depleted (or not) of T cells by injecting i.p. with 250 μg of SFR3 (isotype control) or 2.43 (anti-CD8) in PBS 3 days and 1 day before infection with 5×10⁸ GE of S221 i.v. Liver DENV2 RNA levels on day 3 after infection were measured by qRT-PCR.

FIG. 11 shows a schematic of the basic immunization protocol using the AB6 mouse model of DENV2 infection.

FIG. 12 shows a schematic for varying the immunization protocol.

FIG. 13 shows that adoptively transferred wild-type T cells protect against DENV in AG129 mice.

FIGS. 14A-14D show that DENV2 infection results in CD4+ T cell activation and expansion in IFN-α/βR−/− mice. A) The numbers of splenic CD4+ T cells in naïive IFN-α/βR−/− mice (n=6) and IFN-α/βR−/− mice infected with 10¹⁰ genomic equivalents (GE) of DENV2 (n=11) are shown. ***p<0.001 for naïive versus infected mice. B) The percentage of CD62L^{lo}CD44^{hi} cells (gated on CD4+ cells) is shown for naïve (n=4) and IFN-α/βR−/− mice infected with 1010 GE of DENV2 (n=8). **p<0.01 for naïve versus infected mice. C) Blood lymphocytes were obtained from IFN-α/βR −/− mice on days 3, 5, 7, 10, and 14 after infection with 10¹⁰ GE of DENV2. The percentage of CD44^{hi}CD62L^{lo} cells (gated on CD4+ T cells)±SEM (n=6) is shown. D) The percentage and number of splenic Foxp3+ cells (gated on CD4+ cells) are shown for naïve (n=4) and infected IFN-α/βR−/− mice (n=4).

FIGS. 15A-15B show the identification of DENV2-derived epitopes recognized by CD4⁺ T cells. A) Splenocytes were obtained from IFN-α/βR^{-/-} mice 7 days after infection with 10¹⁰ GE of DENV2 and re-stimulated in vitro with DENV2-derived 15-mer peptides predicted to bind I-A^{b}. Cells were then stained for surface CD4 and intracellular IFN-γ and analyzed by flow cytometry. The 4 positive peptides identified are shown. In the dot plots, the percentage of CD4+ T cells producing IFN-γ is indicated. The responses of individual mice as well as the mean and SEM are also shown (n=7-11). The response of unstimulated cells was subtracted from the response to each DENV2 peptide, and the net percentage and number of splenic CD4⁺ T cells producing IFN-γ are indicated. B) Splenocytes were obtained from wild-type C57BL/6 mice 7 days after infection with 10¹⁰ GE of DENV2 and stimulated and stained as in A (n=6).

FIG. 16 shows that DENV2-specific CD4⁺ T cells are polyfunctional. Splenocytes were obtained from IFN-α/βR^{-/-} mice 7 days after infection with 10¹⁰ GE of DENV2 and stimulated in vitro with individual peptides. Cells were then stained for surface CD4, and intracellular IFN-γ, TNF, IL-2, and CD40L, and analyzed by flow cytometry. The response of unstimulated cells was subtracted from the response to each DENV2 peptide, and the net percentages of the CD4⁺ T cells that are expressing at least one molecule are indicated. The mean and SEM of 3 mice is shown.

FIG. 17 shows that depletion of CD4⁺ T cells prior to DENV2 infection does not affect viral RNA levels. IFN-α/βR^{-/-} mice were depleted of CD4⁺ or CD8⁺ cells, or both, by administration of GK1.5 or 2.43 Ab, respectively, (or given an isotype control Ab) 2 days before and 1 day after infection with 10¹⁰ GE of DENV2. Mice were sacrificed 5 days later, and DENV2 RNA levels in the serum, spleen, small intestine, brain, and kidney were quantified by real-time RT-PCR. Data are expressed as DENV2 copies per ml of sera, or DENV2 units normalized to 18S rRNA levels for the organs. Each symbol represents one mouse, the bar represents the geometric mean, and the dashed line is the limit of detection. *p<0.05, **p<0.01, and ***p<0.001 for viral RNA levels comparing T cell-depleted mice with control mice.

FIGS. 18A-18C show that CD4+ T cells are not required for the anti-DENV2 antibody response. IFN-α/βR^{-/-} mice (control or CD4-depleted) were infected with 10¹⁰ GE of DENV2. A) IgM and IgG titers in the sera at day 7 were measured by ELISA (n=5 control and 6 CD4-depleted mice). Data are combined from two independent studies. B) Neutralizing activity of sera from naïve (n=4) and control (n=6) or CD4-depleted mice (n=6) obtained 7 days after infection was determined by measuring the ability of the sera to reduce DENV2 infection of C6/36 cells. C) The percentage of germinal center B cells (GL7⁺Fas⁺, gated on B220⁺ cells) in the spleen 7 days after infection is shown. The plots are representative of 5 control and 5 CD4-depleted mice.

FIGS. 19A-19C show that CD4⁺ T cells are not required for the primary DENV2-specific CD8⁺ T cell response. A) Splenocytes were obtained from IFN-α/βR^{-/-} mice (control or CD4-depleted) 7 days after infection with 10¹⁰ GE of DENV2, and stimulated in vitro with immunodominant DENV2-derived H-2^{b}-restricted CD8⁺ T cell epitopes. Cells were then stained for CD8 and IFN-g and analyzed by flow cytometry, and the number of CD8⁺ T cells producing IFN-g is shown. Results are expressed as the mean±SEM of 4 mice per group. **p<0.01. B) Splenocytes were obtained as in A and stimulated with NS4B₉₉₋₁₀₇in the presence of an anti-CD107 Ab, and then stained for CD8, IFN-g, TNF, and IL-2. The response of unstimulated cells was subtracted from the response to each DENV2 peptide, and the net percentages of the CD8⁺ T cells that are expressing at least one molecule are indicated. The mean and SEM of 3 mice is shown. C) CD8⁺ T cell-mediated killing. IFN-α/βR^{-/-} mice (control or CD4-depleted) infected 7 days previously with 10¹⁰ GE of DENV2 were injected i.v. with CFSE-labeled target cells pulsed with a pool of DENV2-derived immunodominant H-2^{b}-restricted peptides (C₅₁₋₅₉, NS2A₈₋₁₅, NS4B₉₉₋₁₀₇, and NS5₂₃₇₋₂₄₅) at the indicated concentrations (n=3-6 mice per group). After 4 h, splenocytes were harvested, analyzed by flow cytometry, and the percentage killing was calculated.

FIG. 20 shows cytotoxicity mediated by DENV2-specific CD4⁺ T cells. In vivo killing of DENV2-derived I-A^{b}-restricted peptide-pulsed cells. IFN-α/βR^{-/-} mice (control, CD4-depleted, or CD8-depleted) infected 7 days previously with 10¹⁰ GE of DENV2 were injected i.v. with CFSE-labeled target cells pulsed with the three epitopes that contain only CD4⁺ T cell epitopes (NS2B₁₀₈₋₁₂₂, NS3₁₉₈₋₂₁₂, and NS3₂₃₇₋₅₁) (n=6 control, 3 CD4-depleted, and 3 CD8-depleted mice). After 16 h, splenocytes were harvested, analyzed by flow cytometry, and the percentage killing was calculated.

FIG. 21 shows that peptide immunization with CD4⁺ T cell epitopes results in enhanced DENV2 clearance. IFN-α/βR^{-/-} mice were immunized s.c. with 50 μg each of the three DENV peptides that contain only CD4⁺ T cell epitopes (NS2B₁₀₈₋₁₂₂, NS3₁₉₈₋₂₁₂, NS3₂₃₇₋₅₁) in CFA, or mock-immunized with DMSO in CFA. Mice were boosted 11 days later with peptide in IFA, then challenged with 10¹¹ GE of DENV2 13 days later, and sacrificed 4 days after infection. Separate groups of peptide-immunized mice were depleted of CD4⁺ or CD8⁺ T cells prior to infection. DENV2 RNA levels in the tissues were quantified by real-time RT-PCR and are expressed as DENV2 units normalized to 18S rRNA. Each symbol represents one mouse and the bar represents the geometric mean. *p<0.05, **p<0.01.

FIG. 22A-22D show identification of DENV-derived epitopes recognized by CD8⁺ T cells. DENV specific epitope identification was performed in four different HLA transgenic mouse strains (A) A*0201; (B) A*1101; (C) A*0101; and (D) B*0702. For all strains tested, IFNγ ELISPOT was performed using splenic T cells isolated from HLA transgenic IFN-α/βR^{-/-} mice (black bars) and HLA transgenic IFN-α/βR^{+/+} mice (white bars). Mice were infected i.v. retro-orbitally with 1×10¹⁰ GE of DENV2 (S221) in 100 μl PBS. Seven days post-infection, CD8⁺ T cells were purified and tested against a panel of S221 predicted peptides. The data are expressed as mean number of SFC/10⁶ CD8⁺ T cells of two independent studies. Error bars represent SEM. Responses against peptides were considered positive if the stimulation index (SI) exceeded double the mean negative control wells (effector cells plus APCs without peptide) and net spots were above the threshold of 20 SFCs/10⁶ CD8⁺ T cells in two independent studies. Asterisks indicate peptides, which were able to elicit a significant IFNγ response in each individual study, according to the criteria described above.

FIG. 23 shows identification of DENV-derived epitopes recognized by CD4⁺ T cells. IFNγ ELISPOT was performed using CD4+ T cells isolated from DRB1*0101 transgenic IFN-α/βR^{-/-} (black bars) and IFN-α/βR^{+/+} (white bars) mice. Mice were infected i.v. retro-orbitally with 1×10¹⁰ GE of DENV2 (S221) in 100 μl PBS. Seven days postinfection, CD4⁺ T cells were purified and tested against a panel of S221 predicted peptides. The data are expressed as mean number of SFC/10⁶ CD4+ T cells of two independent studies. Error bars represent SEM. Responses against peptides were considered positive if the stimulation index (SI) exceeded double the mean negative control wells (effector cells plus APCs without peptide) and net spots were above the threshold of 20 SFCs/10⁶ CD4⁺ T cells in two individual studies. Asterisks indicate peptides, which were able to elicit a significant IFNγ response, according to the criteria described above.

FIGS. 24A-24B show the determination of optimal epitope studies. To determine the dominant epitope, HLA-transgenic IFN-α/βR^{-/-} mice were infected with 1×10¹⁰ GE of DENV2 (S221) and spleens harvested 7 days post infection. CD8⁺ T cells were purified and incubated for 24 hours with ascending concentrations of nested peptides. A) shows pairs of peptides where the 9-mer and the 10 mer were able to elicit a significant T cell response; B) shows the 3 B*0702 restricted peptides which did show an IC₅₀>1000 nM in the respective binding assay. Peptides were retested in parallel with their corresponding 8-, 10- and 11-mers. The peptides, which were able to elicit stronger IFNγ responses at various concentrations, were then considered the dominant epitope.

FIGS. 25A-25B show MHC-restriction of identified epitopes. HLA A*0201 (A) and HLA A*1101 (B) transfected 0.221 cells, as well as the non-transfected cell line as a control, were used as antigen presenting cells in titration studies to determine MHC restriction. Purified CD8⁺ T cells from DENV2 (S221) infected HLA A*A0201 and HLA A*110 IFN-α/βR^{-/-} mice were incubated with increasing concentrations of peptides and tested for IFNγ production in an ELISPOT assay. Representative graphs of CD8⁺ T cell responses are shown, when incubated with HLA transfected cell lines (A and B; black lines) and non-transfected cell lines (A and B, grey lines) are shown. The dotted line indicates the 25 net SFCs/10⁶cells threshold used to define positivity.

FIGS. 26A-26F show antigenicity of identified epitopes in human donors. Epitopes (1 μg/ml individual peptide for 7 days) identified in the HLA-transgenic IFN-α/βR^{-/-} mice were validated by their capacity to stimulate PBMC (2×10⁶ PBMC/ml) from human donors and then tested in an IFNγELISPOT assay. A-E) show IFNγ responses/10⁶ PBMC after stimulation with A*0101, A*0201, A*1101, B*0702 and DRB1*0101 restricted peptides, respectively. Donors, seropositive for DENV, were grouped in HLA matched and non-HLA matched cohorts, as shown in panels 1 and 2 of each figure. All epitopes identified were further tested in DENV seronegative individuals. The average IFNγ responses elicited by PBMC from DENV seropositive non-HLA matched and DENV seronegative donors plus 3 times the standard deviation (SD) was set as a threshold for positivity, as indicated by the dashed line. F) shows the mean IFNγ response /10⁶ T cells from HLA transgenic mice (black bars) and HLA matched donors (white bars) grouped by HLA restriction of the epitopes tested.

FIG. 27 shows subprotein location of identified epitopes from Table 2. All identified epitopes were grouped according to the DENV subprotein they are derived from. Black bars show the total IFNγ response all epitopes of a certain protein could elicit. Numbers in parenthesis indicate the number of epitopes that have been detected for this protein.

### DETAILED DESCRIPTION

As disclosed herein, T cells contribute towards protection against primary Dengue virus (DENV) infection in clinically relevant mouse models of Dengue virus (Yauch, et al. J Immunol 185:5405-5416 (2010); Yauch, et al. J Immunol 182:4865-4873 (2009)). The studies disclosed herein demonstrate that CD8+ T cells play a critical role in vaccine-mediated protection against DENV infection. Thus, the findings disclosed herein reveal that CD8+ T cell immunity is required for vaccine-mediated protection against DENV, which is contrary to the general consensus in the field that antibodies are essential for immunization or vaccination against Dengue virus.

Furthermore, the studies disclosed herein demonstrate that the responsive CD8+ T cells after administration of a particular DENV serotype can provide the animal with protection against other distinct (heterologous) DENV serotypes. Thus, the studies disclosed herein reveal that a protein or subsequence of a given DENV serotype can be used to provide protection against other distinct DENV serotypes in vaccination and immunization methods and uses. For example, a DENV3 serotype protein or subsequence or portion can be administered to provide a subject with protection against a DENV1, DENV2 and/or DENV4 serotype infection. Moreover, CD8+ T cells that provide protection against distinct DENV serotypes can also provide protection against other distinct DENV serotypes, even in the presence of enhancing antibodies. Thus, the studies disclosed herein also reveal that a protein or subsequence of a given DENV serotype can be used to provide (broad spectrum) protection in subjects who already have developed antibodies against DENV, as a consequence of a prior DENV infection or exposure to DENV (e.g., vaccination or immunization), for example.

In accordance with the invention, there are provide methods and uses for vaccination and immunization to protect against dengue virus infection, and methods and uses for treatment of a Dengue virus infection. Such methods and uses are applicable to providing a subject with protection from Dengue virus infection, and also are applicable to providing treatment to a subject having a Dengue virus infection, particularly subjects that are at risk of severe dengue disease (e.g., ADE mediated DHF or DSS), such as subjects having Dengue virus antibodies, either produced by their own body due to a prior DENV infection or exposure, or through transfer (e.g., maternal transfer or passive immunization or vaccination with against Dengue virus).

In one embodiment, a use or method for eliciting, stimulating, inducing, promoting, increasing, or enhancing an anti-Dengue virus T cell response in a subject without sensitizing the subject to severe dengue disease upon subsequent Dengue virus infection includes administering to the subject an amount of a Dengue virus protein or subsequence thereof sufficient to elicit, stimulate, induce, promote, increase or enhance an anti-Dengue virus T cell response in the subject.

In another embodiment, a use or method for vaccinating or providing a subject with protection against a Dengue virus infection without eliciting or sensitizing the subject to severe dengue disease upon a secondary or subsequent Dengue virus infection, includes administering to the subject an amount of a Dengue virus protein or subsequence thereof sufficient to vaccinate or provide the subject with protection against the Dengue virus infection.

In another embodiment, a use or method for treating a subject for a Dengue virus infection without eliciting or sensitizing the subject to severe dengue disease (e.g., ADE mediated DHF or DSS) upon a secondary or subsequent Dengue virus infection, includes administering to the subject an amount of a Dengue virus protein or subsequence thereof sufficient to treat the subject for the Dengue virus infection.

As used herein, “sensitize” or “sensitizing” refers to causing a subject to acquire or develop a condition, disease or disorder or the symptoms or complications caused by or associated with the condition, disease or disorder, or to be susceptible to acquiring or developing a condition, disease or disorder or the symptoms or complications cause by or associated with the condition, disease or disorder. In addition, “sensitize” or “sensitizing” may refer to increasing the susceptibility of a subject to acquiring or developing a condition, disease or disorder or the symptoms or complications cause by or associated with the condition, disease or disorder. For example, sensitizing a subject to severe dengue disease upon a secondary or subsequent Dengue virus infection may refer to causing the subject to acquire or develop severe dengue disease or the symptoms or complications caused by or associated with severe dengue disease upon subsequent Dengue virus infection. Sensitizing a subject to severe dengue disease may also refer to causing the subject to be susceptible to acquiring or developing severe dengue disease or one or more other symptoms or complications caused by or associated with severe dengue disease upon a secondary or subsequent Dengue virus infection. In addition, sensitizing a subject to severe dengue disease may also refer to increasing the susceptibility of the subject to acquiring or developing severe dengue disease, one or more other symptoms or complications of severe dengue disease, or more severe symptoms or complications of severe dengue disease, caused by or associated with severe dengue.

A “severe dengue disease” refers to conditions, disease and disorders caused by or associated with Dengue virus infection, including but not limited to dengue hemorrhagic fever (DHF), dengue shock syndrome (DSS) and any symptoms or complications cause by or associated with DHF and DSS including but not limited to increased vascular permeability, thrombocytopenia, hemorrhagic manifestions and death. In certain embodiments, the development of severe dengue disease may be mediated by antibody dependant enhancement (ADE).

As used herein, the term antibody (Ab) dependent enhancement of infection (ADE) refers to a phenomenon in which a subject who has antibodies against Dengue virus, due to a previous Dengue virus infection or exposure to Dengue virus or antigen (e.g., vaccination, immunization, receipt of maternal anti-Dengue virus antibodies, etc.), suffers from enhanced or a more severe illness after a secondary or subsequent infection with a Dengue virus, or after a Dengue virus vaccination or immunization. Typically, the more severe symptoms include one or more of hemorrhagic fever/Dengue shock syndrome, increased viral load, increased vascular permeability, increased hemorrhagic manifestations, thrombocytopenia, and shock, compared to the acute self-limited illness typically caused by Dengue virus in subjects who have not been vaccinated, immunized or previously infected with Dengue virus. Although not wishing to be bound by any theory, ADE is believed to be a consequence of the presence of serotype cross-reactive antibodies enhancing viral infection of FcγR⁺ cells resulting in higher Dengue viral loads and a more severe illness upon subsequent exposure or infection of the subject to a Dengue virus or antigen. Methods and uses of the invention therefore include methods and uses that do not substantially or detectably cause, elicit or stimulate one or more symptoms characteristic of ADE, or more broadly ADE, in a subject.

In addition to ADE, there may be other adverse symptoms that result from, or be enhanced or more severe, when a subject who has antibodies against Dengue virus (e.g., due to a prior infection, exposure, vaccination, immunization, maternal antibodies etc.) becomes infected with Dengue virus, or receives a Dengue virus vaccination or immunization, as compared to a subject that has not been vaccinated, immunized or previously infected with a Dengue virus. Such adverse symptoms that may result from, or may be enhanced or more severe include, for example, fever, headache, rash, liver damage, diarrhea, nausea, vomiting or abdominal pain. It is intended that the methods and uses of the invention therefore also include methods and uses that do not substantially elicit, enhance or worsen one or more such other adverse symptoms that may be elicted, enhanced or be more severe in a subject who has antibodies against a Dengue virus, as compared to a subject that does not have antibodies against a Dengue virus.

A Dengue virus protein of the uses, methods and compositions may be a non-structural or structural Dengue virus protein, subsequence or portion or modification thereof. In certain embodiments, the Dengue virus protein is a non-structural Dengue virus protein, for example, NS1, NS2A, NS2B, NS3, NS4A, NS4B or NS5. In particular embodiments the Dengue virus protein is a NS3, NS4B or NS5 protein, subsequence or portion or modification thereof. In other embodiments, the Dengue virus protein is a structural Dengue virus protein, for example, Dengue virus envelope protein, membrane protein or core protein, subsequence or portion or modification thereof.

As disclosed herein, a DENV protein, subsequence, portion or modification thereof elicits a cellular or humoral immune response. In particular embodiments, a DENV protein, subsequence, portion or modification thereof, elicits, stimulates, promotes or induces a CD8+ T cell and/or CD4+ T cell response. Such responses can provide protection against (e.g., prophylaxis) an initial DENV infection, or a secondary or subsequent DENV infection. Such T cell responses can also be effective in treatment (e.g., therapeutic) of an initial DENV infection, or a secondary or subsequent DENV infection. Such T cell responses can occur without detectably or substantially eliciting, inducing or promoting severe dengue disease (e.g., ADE mediated DHF or DSS) in a subject having anti-DENV antibodies, or detectably or substantially sensitizing a subject to developing severe dengue disease (e.g., ADE mediated DHF or DSS) upon a subsequent DENV infection.

A DENV protein, subsequence, or portion thereof may be derived from or based upon any sequence from any DENV strain or serotype, such as wild-type. Exemplary serotypes are DENV1, DENV2, DENV3 and DENV4. Thus, in various embodiments, a DENV protein, subsequence, portion or modification thereof is derived from or based upon a DENV1, DENV2, DENV3 or DENV4 sequence. More particularly, a protein, subsequence, portion or modification thereof van be derived from or is based upon West Pacific 74 strain (DENV1), UNC 1017 strain (DENV1), UNC 2005 strain (DENV2), S16803 strain (DENV2), UNC 3001 strain (DENV3), UNC 3043 (DENV3, strain 059.AP-2, Philippines), UNC 3009 strain (DENV3, D2863, Sri Lanka), UNC3066 (DENV3, strain 1342 from Puerto Rico 1977), CH 53489 strain (DENV3), TVP-360 (DENV4), or UNC 4019 strain (DENV4). A DENV protein, subsequence, or portion thereof may also be a modified or variant form (hereinafter referred to as a “modification”). Such modified forms, such as amino acid deletions, additions and substitutions, can also be used in the invention uses, methods and compositions for eliciting, inducing, promoting, increasing or enhancing a T cell response, protecting, vaccinating or immunizing a subject, or treatment of a subject, as set forth herein.

As used herein, a subsequence of a Dengue virus protein includes or consists of one or more amino acids less than the full length Dengue virus protein. The term “subsequence” means a fragment or part of the full length molecule. A subsequence of a Dengue virus protein has one or more amino acids less than the full length Dengue virus protein (e.g. one or more internal or terminal amino acid deletions from either amino or carboxy-termini). Subsequences therefore can be any length up to the full length native molecule, provided said length is at least one amino acid less than full length native molecule.

Subsequences can vary in size, for example, from a polypeptide as small as an epitope capable of binding an antibody (i.e., about five amino acids) up to a polypeptide that is one amino acid less than the entire length of a reference polypeptide such as a Dengue virus protein

In various embodiments, a dengue virus protein subsequence is characterized as including or consisting of a NS1 sequence with less than 380 amino acids in length identical to NS1, a NS2A sequence with less than 159 amino acids in length identical to NS2A, a NS2B sequence with less than 130 amino acids in length identical to NS2B, a NS3 sequence with less than 618 amino acids in length identical to NS3, a NS4A sequence with less than 127 amino acids in length identical to NS4A, a NS4B sequence with less than 248 amino acids in length identical to NS4B, a NS5 sequence with less than 900 amino acids in length identical to NS5, a dengue virus envelope protein sequence with less than 495 amino acids in length identical to dengue virus envelope protein, a dengue virus membrane protein sequence with less than 166 amino acids in length identical to dengue virus membrane protein, a dengue virus core protein sequence with less than 96 amino acids in length identical to dengue virus core protein.

Non-limiting exemplary subsequences less than full length NS 1 sequence include, for example, a subsequence from about 5 to 10, 10 to 20, 20 to 30, 30 to 50, 50 to 100, 100 to 150, 150 to 200, 200 to 300, or 300 to 380 amino acids in length. Non-limiting exemplary subsequences less than full length NS2A sequence include, for example, a subsequence from about 5 to 10, 10 to 20, 20 to 30, 30 to 50, 50 to 100, 100 to 159 amino acids in length. Non-limiting exemplary subsequences less than full length NS2B sequence include, for example, a subsequence from about 5 to 10, 10 to 20, 20 to 30, 30 to 50, 50 to 100, 100 to 130 amino acids in length. Non-limiting exemplary subsequences less than full length NS3 sequence include, for example, a subsequence from about 5 to 10, 10 to 20, 20 to 30, 30 to 50, 50 to 100, 100 to 150, 150 to 200, 200 to 300, 300 to 400, 400 to 500, 500 to 618 amino acids in length. Non-limiting exemplary subsequences less than full length NS4A sequence include, for example, a subsequence from about 5 to 10, 10 to 20, 20 to 30, 30 to 50, 50 to 100, 100 to 127 amino acids in length. Non-limiting exemplary subsequences less than full length NS4B sequence include, for example, a subsequence from about 5 to 10, 10 to 20, 20 to 30, 30 to 50, 50 to 100, 100 to 150, 150 to 200 to 248 amino acids in length. Non-limiting exemplary subsequences less than full length NS5 sequence include, for example, a subsequence from about 5 to 10, 10 to 20, 20 to 30, 30 to 50, 50 to 100, 100 to 150, 150 to 200, 200 to 300, 300 to 400, 400 to 500, 500 to 600, 600 to 700, 700 to 800, 800 to 900 amino acids in length. Non-limiting exemplary subsequences less than full length dengue virus envelope protein sequence include, for example, a subsequence from about 5 to 10, 10 to 20, 20 to 30, 30 to 50, 50 to 100, 100 to 150, 150 to 200, 200 to 300, 300 to 400, 400 to 495 amino acids in length. Non-limiting exemplary subsequences less than full length dengue virus membrane protein sequence include, for example, a subsequence from about 5 to 10, 10 to 20, 20 to 30, 30 to 50, 50 to 100, 100 to 150, 150 to 166 amino acids in length. Non-limiting exemplary subsequences less than full length dengue virus core protein sequence include, for example, a subsequence from about 5 to 10, 10 to 20, 20 to 30, 30 to 50, 50 to 96 acids in length.

As used herein, subsequences may also include or consist of one or more amino acid additions or deletions, wherein the subsequence does not comprise the full length native/wild type Dengue virus protein sequence. Accordingly, total subsequence lengths can be greater than the length of the full length native/wild type Dengue virus protein, for example, where a Dengue virus protein subsequence is fused or forms a chimera with another polypeptide.

In other embodiments, the uses, methods and compositions may comprise an Dengue virus protein or peptide comprising or consisting of a subsequence, or an amino acid modification of Dengue virus structural or non-structural protein sequence, wherein the protein or peptide elicits, stimulates, induces, promotes, increases or enhances and anti-Dengue virus CD8⁺ T cell response or an anti-Dengue virus CD4⁺ T cell response, as described herein.

A non-limiting example of a protein, subsequence or portion of a Dengue virus (DV) polypeptide sequence includes or consists of a subsequence or portion of Dengue virus (DV) structural Core, Membrane or Envelope polypeptide sequence. A non-limiting example of a protein, subsequence or portion of a Dengue virus (DV) polypeptide sequence includes or consists of a protein, subsequence or portion of Dengue virus (DV) non-structural (NS) NS1, NS2A, NS2B, NS3, NS4A, NS4B or NS5 polypeptide sequence.

A non-limiting Core sequence of or from which a protein, subsequence, portion or modification can be based upon is a sequence set forth as:

MNNQRKKARNTPFNMLKRERNRVSTVQQLTKRFSLGMLQGRGPLKLFMA
LVAFLRFLTIPPTAGILKRWGTIKKSKAINVLRGFRKEIGRMLNILNRR
RRTAGMIIMLIPTVMA.

A non-limiting Membrane (M) sequence of or from which a protein, subsequence, portion or modification can be based upon is a sequence set forth as:

FHLTTRNGEPHMIVSRQEKGKSLLFKTGDGVNMCTLMAMDLGELCEDTI
TYKCPLLRQNEPEDIDCWCNSTSTWVTYGTCTTTGEHRREKRSVALVPH
VGMGLETRTETWMSSEGAWKHAQRIETWILRHPGFTIMAAILAYTIGTT
HFQRALIFILLTAVAPSMT.

A non-limiting Envelope (E) sequence of or from which a protein, subsequence, portion or modification can be based upon is a sequence set forth as:

MRCIGISNRDFVEGVSGGSWVDIVLEHGSCVTTMAKNKPTLDFELIKTE
AKQSATLRKYCIEAKLTNTTTESRCPTQGEPSLNEEQDKRFVCKHSMVD
RGWGNGCGLFGKGGIVTCAMFTCKKNMKGKVVQPENLEYTIVITPHSGE
EHAVGNDTGKHGKEIKITPQSSITEAELTGYGTVTMECSPRTGLDFNEM
VLLQMENKAWLVHRQWFLDLPLPWLPGADTQGSNWIQKETLVTFKNPHA
KKQDVVVLGSQEGAMHTALTGATEIQMSSGNLLFTGHLKCRLRMDKLQL
KGMSYSMCTGKFKVVKEIAETQHGTIVIRVQYEGDGSPCKIPFEIMDLE
KRHVLGRLITVNPIVTEKDSPVNIEAEPPFGDSYIIIGVEPGQLKLNWF
KKGSSIGQMLETTMRGAKRMAILGDTAWDEGSLGGVFTSIGKALHQVFG
AIYGAAFSGVSWTMKILIGVIITWIGMNSRSTSLSVSLVLVGVVTLYLG
VMVQA.

A non-limiting non-structural NS1 sequence of or from which a protein, subsequence, portion or modification can be based upon is a sequence set forth as:

ADSGCVVSWKNKELKCGSGIFITDNVHTWTEQYKFQPESPSKLASAIQKA
HEEGICGIRSVTRLENLMWKQITPELNHILSENEVKLTIMTGDIKGIMQA
GKRSLRPQPTELKYSWKTWGKAKMLSTESHNQTFLIDGPETAECPNTNRA
WNSLEVEDYGFGVFTTNIWLKLREKQDVFCDSKLMSAAIKDNRAVHADMG
YWIESALNDTWKIEKASFIEVKSCHWPKSHTLWSNEVLESEMIIPKNFAG
PVSQHNYRPGYHTQTAGPWHLGKLEMDFDFCEGTTVVVTEDCGNRGPSLR
TTTASGKLITEWCCRSCTLPPLRYRGEDGCWYGMEIRPLKEKEENLVNSL
VTA.

A non-limiting non-structural NS2A sequence of or from which a protein, subsequence, portion or modification can be based upon is a sequence set forth as:

GHGQIDNFSLGVLGMALFLEEMLRTRVGTKHAILLVAVSFVTLITGNMS
FRDLGRVMVMVGATMTDDIGMGVTYLALLAAFKVRPTFAAGLLLRKLTS
KELMMTTIGIVLLSQSTIPETILELTDALALGMMVLKMVRKMEKYQLAV
TIMAILCVPNAVILQNAWKVSCTILAVVSVSPLFLTSSQQKADWIPLAL
TIKGLNPTAIFLTTLSRTNKKR.

A non-limiting non-structural NS2B sequence of or from which a protein, subsequence, portion or modification can be based upon is a sequence set forth as:

SWPLNEAIMAVGMVSILASSLLKNDIPMTGPLVAGGLLTVCYVLTGRSA
DLELERAADVKWEDQAEISGSSPILSITISEDGSMSIKNEEEEQTLTIL
IRTGLLVISGLFPVSLPITAAAWYLWEVKKQR.

A non-limiting non-structural NS3 sequence of or from which a protein, subsequence, portion or modification can be based upon is a sequence set forth as:

AGVLWDVPSPPPVGKAELEDGAYRIKQKGILGYSQIGAGVYKEGTFHTM
WHVTRGAVLMHKGKRIEPSWADVKKDLISYGGGWKLEGEWKEGEEVQVL
ALEPGKNPRAVQTKPGLEKTNAGTIGAVSLDFSPGTSGSPIIDKKGKVV
GLYGNGVVTRSGAYVSAIAQTEKSIEDNPEIEDDIFRKRKLTIMDLHPG
AGKTKRYLPAIVREAIKRGLRTLILAPTRVVAAEMEEALRGLPIRYQTP
AIRAEHTGREIVDLMCHATFTMRLLSPVRVPNYNLIIMDEAHFTDPASI
AARGYISTRVEMGEAAGIFMTATPPGSRDPFPQSNAPIMDEEREIPERS
WSSGHEWVTDFKGKTVWFVPSIKAGNDIAACLRKNGKKVIQLSRKTEDS
EYVKTRTNDWDFVVTTDISEMGANFKAERVIDPRRCMKPVILTDGEERV
ILAGPMPVTHSSAAQRRGRIGRNPKNENDQYIYMGEPLENDEDCAHWKE
AKMLLDNINTPEGIIPSMFEPEREKVDAIDGEYRLRGEARKTFVDLMRR
GDLPVWLAYRVAAEGINYADRRWCFDGIKNNQILEENVEVEIWTKEGER
KKLKPRWLDARIYSDPLALKEFKEFAAGRK.

A non-limiting non-structural NS4A sequence of or from which a protein, subsequence, portion or modification can be based upon is a sequence set forth as:

SLTLSLITEMGRLPTFMTQKARDALDNLAVLHTAEAGGRAYNHALSELPE
TLETLLLLTLLATVTGGIFLFLMSGRGIGKMTLGMCCIITASILLWYAQI
QPHWIAASIILEFFLIVLLIPEPEKQRTPQDNQLTYVVIAILTVVAATMA.

A non-limiting non-structural NS4B sequence of or from which a protein, subsequence, portion or modification can be based upon is a sequence set forth as:

NEMGFLEKTKKDLGLGSITTQQPESNILDIDLRPASAWTLYAVATTFVTP
MLRHSIENSSVNVSLTAIANQATVLMGLGKGWPLSKMDIGVPLLAIGCYS
QVNPITLTAALFLLVAHYAIIGPGLQAKATREAQKRAAAGIMKNPTVDGI
TVIDLDPIPYDPKFEKQLGQVMLLVLCVTQVLMMRTTWALCEALTLATGP
ISTLWEGNPGRFWNTTIAVSMANIFRGSYLAGAGLLFSIMKNTTNTRR.

A non-limiting non-structural NS5 sequence of or from which a protein, subsequence, portion or modification can be based upon is a sequence set forth as:

GTGNIGETLGEKWKSRLNALGKSEFQIYKKSGIQEVDRTLAKEGIKRGET
DHHAVSRGSAKLRWFVERNMVTPEGKVVDLGCGRGGWSYYCGGLKNVREV
KGLTKGGPGHEEPIPMSTYGWNLVRLQSGVDVFFTPPEKCDTLLCDIGES
SPNPTVEAGRTLRVLNLVENWLNNNTQFCIKVLNPYMPSVIEKMEALQRK
YGGALVRNPLSRNSTHEMYWVSNASGNIVSSVNMISRMLINRFTMRHKKA
TYEPDVDLGSGTRNIGIESEIPNLDIIGKRIEKIKQEHETSWHYDQDHPY
KTWAYHGSYETKQTGSASSMVNGVVRLLTKPWDVVPMVTQMAMTDTTPFG
QQRVFKEKVDTRTQEPKEGTKKLMKITAEWLWKELGKKKTPRMCTREEFT
RKVRSNAALGAIFTDENKWKSAREAVEDSRFWELVDKERNLHLEGKCETC
VYNMMGKREKKLGEFGKAKGSRAIWYMWLGARFLEFEALGFLNEDHWFSR
ENSLSGVEGEGLHKLGYILRDVSKKEGGAMYADDTAGWDTRITLEDLKNE
EMVTNHMEGEHKKLAEAIFKLTYQNKVVRVQRPTPRGTVMDIISRRDQRG
SGQVGTYGLNTFTNMEAQLIRQMEGEGVFKSIQHLTVTEEIAVQNWLARV
GRERLSRMAISGDDCVVKPLDDRFASALTALNDMGKVRKDIQQWEPSRGW
NDWTQVPFCSHHFHELIMKDGRVLVVPCRNQDELIGRARISQGAGWSLRE
TACLGKSYAQMWSLMYFHRRDLRLAANAICSAVPSHWVPTSRTTWSIHAK
HEWMTAEDMLTVWNRVWIQENPWMEDKTPVESWEEIPYLGKREDQWCGSL
IGLTSRATWAKNIQTAINQVRSLIGNEEYTDYMPSMKRFRREEEEAGVLW.

Structural proteins E and prM are major targets of anti-DENV antibody response. NS proteins (in particular NS3, NS4B and NS5) are more conserved across the four DENV serotypes than E, and NS proteins are not expressed in DENV virions (unlike E and PrM proteins). Thus, without being limited to any particular theory, it appears that NS3, NS4B, or NS5 will be better at inducing cross-protective (heterologous) CD8+ T cell responses and at avoiding ADE. Thus without being limited to or bound by any particular theory, DENV vaccines expressing NS3, NS4B, or NS5 will likely provide superior CD8+ T cell immunity against DENV infection, or secondary or subsequent infection (reinfection) than Envelope and Membrane proteins.

As disclosed herein, Dengue virus (DV) proteins, subsequences, portions and modifications thereof of the invention include those having all or at least partial sequence identity to one or more exemplary Dengue virus (DV) proteins, subsequences, portions or modifications thereof (e.g., sequences set forth in Tables 1-4). The percent identity of such sequences can be as little as 60%, or can be greater (e.g., 65%, 70%, 75%, 80%, 85%, 90%, 95%, 96%, 97%, 98%, 99%, etc.). The percent identity can extend over the entire sequence length or a portion of the sequence. In particular aspects, the length of the sequence sharing the percent identity is 2, 3, 4, 5 or more contiguous amino acids, e.g., 6, 7, 8, 9, 10, 11, 12, 13, 14, 15, 16, 17, 18, 19, 20, etc. contiguous amino acids. In additional particular aspects, the length of the sequence sharing the percent identity is 20 or more contiguous amino acids, e.g., 20, 21, 22, 23, 24, 25, 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet further particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The term “identity” and grammatical variations thereof, mean that two or more referenced entities are the same. Thus, where two Dengue virus (DV) proteins, subsequences, portions and modifications thereof are identical, they have the same amino acid sequence. The identity can be over a defined area (region or domain) of the sequence. “Areas, regions or domains” of homology or identity mean that a portion of two or more referenced entities share homology or are the same.

The extent of identity between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

In accordance with the invention, modified and variant forms of Dengue virus (DV) proteins, subsequences and portions there are provided. Such forms, referred to as “modifications” or “variants” and grammatical variations thereof, are a Dengue virus (DV) protein, subsequence or portion thereof that deviates from a reference sequence. For example, certain sequences set forth in Tables 1-4 are considered a modification or variant of Dengue virus (DV) protein, subsequence or portion thereof. Such modifications may have greater or less activity or function than a reference Dengue virus (DV) protein, subsequence or portion thereof, such as ability to elicit, stimulate, induce, promote, increase, enhance or activate a CD4⁺ or a CD8⁺ T cell response. Thus, Dengue virus (DV) proteins, subsequences and portions thereof include sequences having substantially the same, greater or less relative activity or function as a T cell epitope than a reference T cell epitope (e.g., any of the sequences in Tables 1-4), for example, an ability to elicit, stimulate, induce, promote, increase, enhance or activate an anti-DV CD4⁺ T cell or anti-DV CD8⁺ T cell response in vitro or in vivo.

Non-limiting examples of modifications include one or more amino acid substitutions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues), additions and insertions (e.g., 1, 2, 3, 4, 5, 6, 7, 8, 9, 10, 1, 12, 13, 14, 15, 16, 17, 18, 19, 20, 20-25, 25-30, 30-50, 50-100, or more residues) and deletions (e.g., 1, 2, 3, 4, 5, 6, 7, 8, 9, 10, 1, 12, 13, 14, 15, 16, 17, 18, 19, 20, 20-25, 25-30, 30-50, 50-100) of a reference Dengue virus (DV) protein, subsequence or portion thereof. In particular embodiments, a modified or variant sequence retains at least part of a function or an activity of unmodified sequence, and can have less than, approximately the same, or greater, but at least a part of, a function or activity of a reference sequence, for example, the ability to elicit, stimulate, induce, promote, increase, enhance or activate an anti-DV CD4⁺ T cell or anti-DV CD8⁺ T cell response in vitro or in vivo. Such CD4⁺ T cell and CD8⁺ T cell responses elicited include, for example, among others, induced, increased, enhanced, stimulate or activate expression or production of a cytokine (e.g., IFN-gamma, TNF, IL-2 or CD40L), release of a cytotoxin (perforin or granulysin), or apoptosis of a target (e.g., DV infected) cell.

Specific non-limiting examples of substitutions include conservative and non-conservative amino acid substitutions. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge, or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

An addition can be the covalent or non-covalent attachment of any type of molecule to the sequence. Specific examples of additions include glycosylation, acetylation, phosphorylation, amidation, formylation, ubiquitination, and derivatization by protecting/blocking groups and any of numerous chemical modifications. Additional specific non-limiting examples of an addition are one or more additional amino acid residues. Accordingly, DV sequences including DENV proteins, T cell epitopes, subsequences, portions, and modifications thereof can be a part of or contained within a larger molecule, such as another protein or peptide sequence, such as a fusion or chimera with a different DV sequence, or a non-DV protein or subsequence or portion or modification thereof. In particular embodiments, an addition is a fusion (chimeric) sequence, an amino acid sequence having one or more molecules not normally present in a reference native (wild type) sequence covalently attached to the sequence.

The term “chimeric” and grammatical variations thereof, when used in reference to a sequence, means that the sequence contains one or more portions that are derived from, obtained or isolated from, or based upon other physical or chemical entities. For example, a chimera of two or more different proteins may have one part a Dengue virus (DV) peptide, subsequence, portion or modification, and a second part of the chimera may be from a different Dengue virus (DV) protein sequence, or a non-Dengue virus (DV) sequence.

Another particular example of a modified sequence having an amino acid addition is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), and radioisotope. For example, a tag such as T7 or polyhistidine can be attached in order to facilitate purification or detection of a T cell epitope. Thus, in other embodiments, the invention provides Dengue virus (DV) proteins, subsequences, portions and modifications thereof and a heterologous domain, wherein the heterologous functional domain confers a distinct function, on the Dengue virus (DV) proteins, subsequences, portions and modifications thereof. Such constructs containing Dengue virus (DV) proteins, subsequences, portions and modifications thereof and a heterologous domain are also referred to as chimeras.

Linkers, such as amino acid or peptidomimetic sequences may be inserted between the sequence and the addition (e.g., heterologous functional domain) so that the two entities maintain, at least in part, a distinct function or activity. Linkers may have one or more properties that include a flexible conformation, an inability to form an ordered secondary structure or a hydrophobic or charged character, which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329). Linkers further include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further non-limiting examples of additions are detectable labels. Thus, in another embodiment, the invention provides Dengue virus (DV) proteins, subsequences and portions thereof that are detectably labeled. Specific examples of detectable labels include fluorophores, chromophores, radioactive isotopes (e.g., S³⁵, P³², I¹²⁵), electron-dense reagents, enzymes, ligands and receptors. Enzymes are typically detected by their activity. For example, horseradish peroxidase is usually detected by its ability to convert a substrate such as 3,3-′,5,5-′-tetramethylbenzidine (TMB) to a blue pigment, which can be quantified.

Another non-limiting example of an addition is an insertion of an amino acid within any Dengue virus (DV) protein, subsequence, portion or modification thereof (e.g., any DV sequence set forth herein, such as in Tables 1-4). In particular embodiments, an insertion is of one or more amino acid residues inserted into a Dengue virus (DV) protein, subsequence portion or modification thereof, such as any sequence set forth herein, such as in Tables 1-4.

Modified and variant Dengue virus (DV) proteins, subsequences and portions thereof also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond. Dengue virus (DV) proteins, subsequences and portions thereof may be modified in vitro or in vivo, e.g., post-translationally modified to include, for example, sugar residues, phosphate groups, ubiquitin, fatty acids, lipids, etc.

Specific non-limiting examples of Dengue virus proteinsubsequences or portions include an amino acid sequence comprising at least one amino acid deletion from full length Dengue virus (DV) protein sequence. In particular embodiments, a protein subsequence or portion is from about 5 to 300 amino acids in length, provided that said subsequence or portion is at least one amino acid less in length than the full-length Dengue virus (DV) structural sequence or the non-structural (NS) sequence. In additional particular embodiments, a protein subsequence or portion is from about 2 to 5, 5 to 10, 10 to 15, 15 to 20, 20 to 25, 25 to 50, 50 to 100, 100 to 150, 150 to 200, or 200 to 300 amino acids in length, provided that said subsequence or portion is at least one amino acid less in length than the full-length Dengue virus (DV) structural protein sequence or non-structural (NS) protein sequence.

Dengue virus (DV) proteins, subsequences and portions thereof including modified forms can be produced by any of a variety of standard protein purification or recombinant expression techniques. For example, a Dengue virus (DV) protein, subsequence, portion or modification thereof can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Dengue virus (DV) proteins, subsequences and portions thereof including modified forms can be made using recombinant DNA technology via cell expression or in vitro translation. Polypeptide sequences including modified forms can also be produced by chemical synthesis using methods known in the art, for example, an automated peptide synthesis apparatus (see, e.g., Applied Biosystems, Foster City, Calif.).

The invention provides isolated and/or purified Dengue virus (DV) proteins, including or consisting of a protein, subsequence, portion or modification of a structural core (C), membrane (M) or envelope (E) polypeptide sequence, or a non-structural (NS) NS1, NS2A, NS2B, NS3, NS4A, NS4B or NS5 polypeptide sequence. In particular embodiments, an isolated and/or purified protein, subsequence, portion or modification of the Dengue virus (DV) polypeptide sequence includes a T cell epitope, e.g., as set forth in Tables 1-4.

The term “isolated,” when used as a modifier of a composition (e.g., Dengue virus (DV) proteins, subsequences, portions and modifications thereof, nucleic acids encoding same, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., Dengue virus (DV) protein, subsequence, portion or modification thereof) can also be “substantially pure” or “purified” when free of most or all of the materials with which it typically associates with in nature. Thus, an isolated Dengue virus (DV) protein, subsequence, portion or modification thereof, that also is substantially pure or purified does not include polypeptides or polynucleotides present among millions of other sequences, such as peptides of an peptide library or nucleic acids in a genomic or cDNA library, for example.

A “substantially pure” or “purified” composition can be combined with one or more other molecules. Thus, “substantially pure” or “purified” does not exclude combinations of compositions, such as combinations of Dengue virus (DV) proteins, subsequences, portions and modifications thereof (e.g., multiple, T cell epitopes), and other antigens, agents, drugs or therapies.

The invention also provides nucleic acids encoding Dengue virus (DV) proteins, subsequences, portions and modifications thereof. Such nucleic acid sequences encode a sequence at least 60% or more (e.g., 65%, 70%, 75%, 80%, 85%, 90%, 95%, etc.) identical to a Dengue virus (DV) protein, subsequence or portion thereof. In an additional embodiment, a nucleic acid encodes a sequence having a modification, such as one or more amino acid additions (insertions), deletions or substitutions of a Dengue virus (DV) protein, subsequence or portion thereof, such as any sequence set forth in Tables 1-4.

The terms “nucleic acid,” “polynucleotide” and “polynucleoside” and the like refer to at least two or more ribo- or deoxy-ribonucleic acid base pairs (nucleotides/nucleosides) that are linked through a phosphoester bond or equivalent. Nucleic acids include polynucleotides and polynucleosides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 bases to 20 Kilobases (Kb), or any numerical value or range within or encompassing such lengths, 10 bases to 10Kb, 1 to 5 Kb or less, 1000 to about 500 bases or less in length. Nucleic acids can also be shorter, for example, 100 to about 500 bases, or from about 12 to 25, 25 to 50, 50 to 100, 100 to 250, or about 250 to 500 bases in length, or any numerical value or range or value within or encompassing such lengths. In particular aspects, a nucleic acid sequence has a length from about 10-20, 20-30, 30-50, 50-100, 100-150, 150-200, 200-250, 250-300, 300-400, 400-500, 500-1000, 1000-2000 bases, or any numerical value or range within or encompassing such lengths. Shorter nucleic acids are commonly referred to as “oligonucleotides” or “probes” of single- or double-stranded DNA. However, there is no upper limit to the length of such oligonucleotides.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode Dengue virus (DV) proteins, subsequences and portions thereof, as well as variants and modifications thereof (e.g., substitutions, additions, insertions and deletions).

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to the encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as eukaryote or mammalian cell, yeast or bacteria, in an animal or in a plant).

Nucleic acid may be inserted into a nucleic acid construct in which expression of the nucleic acid is influenced or regulated by an “expression control element.” An “expression control element” refers to a nucleic acid sequence element that regulates or influences expression of a nucleic acid sequence to which it is operatively linked. Expression control elements include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. Expression control elements include elements that activate transcription constitutively, that are inducible (i.e., require an external signal for activation), or derepressible (i.e., require a signal to turn transcription off; when the signal is no longer present, transcription is activated or “derepressed”), or specific for cell-types or tissues (i.e., tissue-specific control elements).

Nucleic acid can also be inserted into a plasmid for propagation into a host cell and for subsequent genetic manipulation. A plasmid is a nucleic acid that can be propagated in a host cell, plasmids may optionally contain expression control elements in order to drive expression of the nucleic acid encoding Dengue virus (DV) proteins, subsequences, portions and modifications thereof in the host cell. A vector is used herein synonymously with a plasmid and may also include an expression control element for expression in a host cell (e.g., expression vector). Plasmids and vectors generally contain at least an origin of replication for propagation in a cell and a promoter. Plasmids and vectors are therefore useful for genetic manipulation and expression of Dengue virus (DV) proteins, subsequences and portions thereof. Accordingly, vectors that include nucleic acids encoding or complementary to Dengue virus (DV) proteins, subsequences, portions and modifications thereof, are provided.

In accordance with the invention, there are provided particles (e.g., viral particles) and transformed host cells that express and/or are transformed with a nucleic acid that encodes and/or express Dengue virus (DV) proteins, subsequences, portions and modifications thereof. Particles and transformed host cells include but are not limited to virions, and prokaryotic and eukaryotic cells such as bacteria, fungi (yeast), plant, insect, and animal (e.g., mammalian, including primate and human, CHO cells and hybridomas) cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression. The cells may be a primary cell isolate, cell culture (e.g., passaged, established or immortalized cell line), or part of a plurality of cells, or a tissue or organ ex vivo or in a subject (in vivo).

The term “transformed” or “transfected” when used in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid or protein can be stably or transiently transfected or transformed (expressed) in the host cell and progeny thereof. The cell(s) can be propagated and the introduced protein expressed, or nucleic acid transcribed. A progeny of a transfected or transformed cell may not be identical to the parent cell, since there may be mutations that occur during replication.

Expression of Dengue virus (DV) proteins, subsequences, portions and modifications thereof, and nucleic acid in particles or introduction into target cells (e.g., host cells) can also be carried out by methods known in the art. Non-limiting examples include osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means delivery into cells are included.

Dengue virus proteins, subsequences, portions and modifications thereof can be employed in various methods and uses. Such methods and uses include, for example, use, contact or administration of one or more DENV proteins, subsequences or modifications thereof, such as the proteins and subsequences set forth herein (e.g., Tables 1-4), in vitro and in vivo.

In accordance with the invention, there are provided methods for eliciting, stimulating, inducing, promoting, increasing, or enhancing an anti-Dengue virus T cell response in a subject without sensitizing the subject to severe dengue disease upon subsequent Dengue virus infection, the method comprising administering to the subject an amount of a Dengue virus protein or subsequence thereof sufficient to elicit an anti-Dengue virus T cell response in the subject.

In another aspect, there is provided a method for providing a subject with protection against a Dengue virus infection or pathology, or one or more physiological disorders, illness, diseases or symptoms caused by or associated with Dengue virus infection or pathology without sensitizing the subject to severe dengue disease upon subsequent Dengue virus infection, the method comprising administering to the subject an amount of a Dengue virus protein or subsequence thereof sufficient to protect the subject against Dengue virus infection.

In yet another aspect of the invention, there is provided a method of vaccinating a subject against a Dengue virus infection without sensitizing the subject to severe dengue disease upon subsequent Dengue virus infection, the method comprising administering to the subject an amount of a Dengue virus protein or subsequence thereof sufficient to vaccinate the subject against the Dengue virus infection.

In a further aspect of the invention, there is provided a method of treating a subject for a Dengue virus infection without sensitizing the subject to severe dengue disease upon subsequent Dengue virus infection, the method comprising administering to the subject an amount of a Dengue virus protein or subsequence thereof sufficient to treat the subject for the Dengue virus infection.

As used herein, the terms “protect” and grammatical variations thereof, when used in reference to a Dengue virus infection or pathology, means preventing a DENV infection, or reducing or decreasing susceptibility to a DENV infection, or preventing or reducing one or more symptoms or pathologies caused by or associated with DENV infection or pathology, such as ADE. A subject may be protected from one or more DENV serotypes, e.g. any or all of DENV 1, 2, 3 or 4, or any variant serotype. A protected subject may also have been previously exposed to or infected with a DENV, and have developed antibodies against DENV. Protection in this context would therefore include, but not be limited to, protection from a secondary or subsequent DENV infection.

In accordance with the invention, uses and methods of stimulating, inducing, promoting, increasing, or enhancing an immune response against Dengue virus (DV) in a subject are provided. In one embodiment, a method includes administering to a subject an amount of a Dengue virus (DV) protein, subsequence or portion or modification thereof, such as a T cell epitope, sufficient to stimulate, induce, promote, increase, or enhance an immune response against Dengue virus (DV) in the subject. Such immune response methods can in turn be used to provide a subject with protection against a Dengue virus (DV) infection or pathology, or one or more physiological conditions, disorders, illness, diseases or symptoms caused by or associated with DV infection or pathology.

In accordance with the invention, treatment uses and methods are provided that include therapeutic (following Dengue virus (DV) infection) and prophylactic (prior to Dengue virus (DV) exposure, infection or pathology) uses and methods. For example, therapeutic and prophylactic uses and methods of treating a subject for a Dengue virus (DV) infection include but are not limited to treatment of a subject having or at risk of having a Dengue virus (DV) infection or pathology, treating a subject with a Dengue virus (DV) infection, and methods of protecting a subject from a Dengue virus (DV) infection (e.g., provide the subject with protection against Dengue virus (DV) infection), to decrease or reduce the probability of a Dengue virus (DV) infection in a subject, to decrease or reduce susceptibility of a subject to a Dengue virus (DV) infection, to inhibit or prevent a Dengue virus (DV) infection in a subject, and to decrease, reduce, inhibit or suppress transmission of the Dengue virus (DV) from a host (e.g., a mosquito) to a subject.

Such methods include, for example, administering Dengue virus (DV) protein, subsequence, portion or modification thereof to therapeutically or prophylactically treat (vaccinate or immunize) a subject having or at risk of having a Dengue virus (DV) infection or pathology. Accordingly, uses and methods can treat a Dengue virus (DV) infection or pathology, or provide a subject with protection from infection (e.g., prophylactic protection).

In one embodiment, a method includes administering to a subject an amount of Dengue virus (DV) protein, subsequence, portion or modification thereof sufficient to treat the subject for the Dengue virus (DV) infection or pathology. In another embodiment, a method includes administering to a subject an amount of a Dengue virus (DV) protein, subsequence, portion or modification sufficient to provide the subject with protection against the Dengue virus (DV) infection or pathology, or one or more physiological conditions, disorders, illness, diseases or symptoms caused by or associated with the virus infection or pathology. In a further embodiment, a method includes administering a subject an amount of a Dengue virus (DV) protein, subsequence, portion or modification sufficient to treat the subject for the Dengue virus (DV) infection.

Dengue virus (DV) proteins, subsequences, portions and modifications thereof include T cell epitopes. In one embodiment, a method includes administering an amount of Dengue virus (DV) protein, subsequence, portion or modification thereof (e.g., a T cell epitope) to a subject in need thereof, sufficient to provide the subject with protection against Dengue virus (DV) infection or pathology. In another embodiment, a method includes administering an amount of a Dengue virus (DV) protein, subsequence, portion or modification thereof (e.g., a T cell epitope) to a subject in need thereof sufficient to treat, vaccinate or immunize the subject against the Dengue virus (DV) infection or pathology.

In accordance with the invention, uses and methods of inducing, increasing, promoting or stimulating anti-Dengue virus (DV) activity of CD8⁺ T cells or CD4⁺ T cells in a subject are provided. In one embodiment, a method includes administering to a subject an amount of a Dengue virus (DV) protein, subsequence or portion, or modification thereof, such as a T cell epitope, sufficient to induce, increase, promote or stimulate anti-Dengue virus (DV) activity of CD8⁺ T cells or CD4⁺ T cells in the subject.

In methods of the invention, any appropriate Dengue virus (DV) protein, subsequence, portion or modification thereof can be used or administered. Non-limiting examples include Dengue virus (DV) protein, subsequence, portion or modification thereof of a DENV1, DENV2, DENV3 or DENV4 serotype protein, subsequence or portion or modification thereof, such as a T cell epitope. Additional non-limiting examples include a Dengue virus structural protein (e.g., C, M or E) or non-structural (NS) protein (e.g., NS1, NS2A, NS2B, NS3, NS4A, NS4B or NS5), or a subsequence or portion or modification thereof, such as a T cell epitope, in or of such structural and non-structural (NS) proteins. Particular non-limiting examples include a DENV protein, or a protein subsequence, such as a sequence set forth in Tables 1-4, or a subsequence or a modification thereof.

In particular uses and methods embodiments, one or more disorders, diseases, physiological conditions, pathologies and symptoms associated with or caused by a Dengue virus (DV) infection or pathology will respond to treatment. In particular methods embodiments, treatment uses and methods reduce, decrease, suppress, limit, control or inhibit Dengue virus (DV) numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a Dengue virus (DV) nucleic acid. In additional particular uses and methods embodiments, treatment uses and methods include an amount of a Dengue virus (DV) protein, subsequence or portion or modification thereof sufficient to increase, induce, enhance, augment, promote or stimulate an immune response against a Dengue virus (DV); increase, induce, enhance, augment, promote or stimulate Dengue virus (DV) clearance or removal; or decrease, reduce, inhibit, suppress, prevent, control, or limit transmission of Dengue virus (DV) to a subject (e.g., transmission from a host, such as a mosquito, to a subject). In further particular uses and methods embodiments, treatment uses and methods include an amount of Dengue virus (DV) protein, subsequence or portion or modification thereof sufficient to protect a subject from a Dengue virus (DV) infection or pathology, or reduce, decrease, limit, control or inhibit susceptibility to Dengue virus (DV) infection or pathology.

Uses and methods of the invention include treatment uses and methods, which result in any therapeutic or beneficial effect. In various methods embodiments, Dengue virus (DV) infection, proliferation or pathogenesis is reduced, decreased, inhibited, limited, delayed or prevented, or a use or method decreases, reduces, inhibits, suppresses, prevents, controls or limits one or more adverse (e.g., physical) symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In additional various particular embodiments, treatment uses and methods include reducing, decreasing, inhibiting, delaying or preventing onset, progression, frequency, duration, severity, probability or susceptibility of one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In further various particular embodiments, treatment uses and methods include improving, accelerating, facilitating, enhancing, augmenting, or hastening recovery of a subject from a Dengue virus (DV) infection or pathogenesis, or one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In yet additional various embodiments, treatment uses and methods include stabilizing infection, proliferation, replication, pathogenesis, or an adverse symptom, disorder, illness, disease or complication caused by or associated with Dengue virus (DV) infection, proliferation or replication, or pathology, or decreasing, reducing, inhibiting, suppressing, limiting or controlling transmission of Dengue virus (DV) from a host (e.g., mosquito) to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with Dengue virus (DV) infection, proliferation or replication, or pathology (e.g., fever, rash, headache, pain behind the eyes, muscle or joint pain, nausea, vomiting, loss of appetite). Thus, a satisfactory clinical endpoint is achieved when there is an incremental improvement or a partial reduction in an adverse symptom, disorder, illness, disease or complication caused by or associated with Dengue virus (DV) infection, proliferation or replication, or pathology, or an inhibition, decrease, reduction, suppression, prevention, limit or control of worsening or progression of one or more adverse symptoms, disorders, illnesses, diseases or complications caused by or associated with Dengue virus (DV) infection, Dengue virus (DV) numbers, titers, proliferation or replication, Dengue virus (DV) protein or nucleic acid, or Dengue virus (DV) pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a Dengue virus (DV) infection or pathology. For example, reducing an amount of an adjunct therapy, for example, a reduction or decrease of a treatment for a Dengue virus (DV) infection or pathology, or a vaccination or immunization protocol is considered a beneficial effect. In addition, reducing or decreasing an amount of a Dengue virus (DV) antigen used for vaccination or immunization of a subject to provide protection to the subject is considered a beneficial effect.

Adverse symptoms and complications associated with Dengue virus (DV) infection and pathology include, for example, e.g., fever, rash, headache, pain behind the eyes, muscle or joint pain, nausea, vomiting, loss of appetite, etc. Thus, the aforementioned symptoms and complications are treatable in accordance with the invention. Other symptoms of Dengue virus (DV) infection and pathology include ADE, which occurs upon a secondary or subsequent DENV infection of a subject, which had been previously infected with or exposed to DENV. ADE, as set forth herein or known to one of skill in the art, can be minimized or avoided (i.e., a subject would not be sensitized to ADE), or ADE would not be substantially elicited, induced, stimulated or promoted in a subject, in accordance with the invention uses and methods. Additional symptoms of Dengue virus (DV) infection or pathogenesis are known to one of skill in the art and treatment thereof in accordance with the invention is provided.

Uses, methods and compositions of the invention also include increasing, stimulating, promoting, enhancing, inducing or augmenting an anti-DENV CD4⁺ and/or CD8⁺ T cell responses in a subject, such as a subject with or at risk of a Dengue virus infection or pathology. In one embodiment, a use or method includes administering to a subject an amount of Dengue virus (DV) protein, subsequence, portion or modification thereof sufficient to increase, stimulate, promote, enhance, augment or induce anti-DENV CD4+ or CD8⁺T cell response in the subject. In another embodiment, a method includes administering to a subject an amount of Dengue virus (DV) protein, subsequence, portion or modification thereof, and administering a Dengue virus (DV) antigen, live or attenuated Dengue virus (DV), or a nucleic acid encoding all or a portion (e.g., a T cell epitope) of any protein or proteinaceous Dengue virus (DV) antigen sufficient to increase, stimulate, promote, enhance, augment or induce anti-Dengue virus (DV) CD4⁺ T cell or CD8⁺ T cell response in the subject.

Uses and methods of the invention additionally include, among other things, increasing production of a Th1 cytokine (e.g., IFN-gamma, TNF-alpha, IL-1alpha, IL-2, IL-6, IL-8, etc.) or other signaling molecule (e.g., CD40L) in vitro or in vivo. In one embodiment, a method includes administering to a subject in need thereof an amount of Dengue virus (DV) protein, subsequence or portion or modification thereof sufficient to increase production of a Th1 cytokine in the subject (e.g., IFN-gamma, TNF-alpha, IL-lalpha, IL-2, IL-6, IL-8, etc.) or other signaling molecule (e.g., CD40L).

Uses, methods and compositions of the invention include administration of Dengue virus (DV) protein, subsequence, portion or modification thereof to a subject prior to contact, exposure or infection by a Dengue virus, administration prior to, substantially contemporaneously with or after a subject has been contacted by, exposed to or infected with a Dengue virus (DV), and administration prior to, substantially contemporaneously with or after Dengue virus (DV) pathology or development of one or more adverse symptoms. Methods, compositions and uses of the invention also include administration of Dengue virus (DV) protein, subsequence, portion or modification thereof to a subject prior to, substantially contemporaneously with or following an adverse symptom, disorder, illness or disease caused by or associated with a Dengue virus (DV) infection, or pathology. A subject infected with a Dengue virus (DV) may have an infection over a period of 1-5, 5-10, 10-20, 20-30, 30-50, 50-100 hours, days, months, or years.

Invention compositions (e.g., Dengue virus (DV) protein, subsequence or portion or modification thereof, including T cell epitopes) and uses and methods can be combined with any compound, agent, drug, treatment or other therapeutic regimen or protocol having a desired therapeutic, beneficial, additive, synergistic or complementary activity or effect. Exemplary combination compositions and treatments include multiple DENV proteins, subsequences, portions or modifications thereof, such as T cell epitopes as set for the herein, second actives, such as anti-Dengue virus (DV) compounds, agents and drugs, as well as agents that assist, promote, stimulate or enhance efficacy. Such anti-Dengue virus (DV) drugs, agents, treatments and therapies can be administered or performed prior to, substantially contemporaneously with or following any other use or method of the invention, for example, a therapeutic use or method of treating a subject for a Dengue virus (DV) infection or pathology, or a use or method of prophylactic treatment of a subject for a Dengue virus (DV) infection.

Dengue virus (DV) proteins, subsequences, portions and modifications thereof can be administered as a combination composition, or administered separately, such as concurrently or in series or sequentially (prior to or following) administering a second active, to a subject. The invention therefore provides combinations in which a use or method of the invention is in a combination with any compound, agent, drug, therapeutic regimen, treatment protocol, process, remedy or composition, such as an anti-viral (e.g., Dengue virus (DV)) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of one or more Dengue virus (DV) proteins, subsequences, portions or modifications thereof, or a nucleic acid encoding all or a portion (e.g., a T cell epitope) of a Dengue virus (DV) protein, subsequence, portion or modification thereof, to a subject. Specific non-limiting examples of combination embodiments therefore include the foregoing or other compound, agent, drug, therapeutic regimen, treatment protocol, process, remedy or composition.

An exemplary combination is a Dengue virus (DV) protein, subsequence, portion or modification thereof (e.g., a CD4⁺ or CD8⁺ T cell epitope) and a different Dengue virus (DV) protein, subsequence, portion or modification thereof (e.g., a different T cell epitope) such as a DENV protein or T cell epitope, antigen (e.g., Dengue virus (DV) extract), or live or attenuated Dengue virus (DV) (e.g., inactivated Dengue virus (DV)). Another exemplary combination is a Dengue virus (DV) protein, subsequence, portion or modification thereof and a T-cell stimulatory molecule, including for example an OX40 or CD27 agonist.

Such Dengue virus (DV) proteins, antigens and T cell epitopes set forth herein or known to one skilled in the art include Dengue virus (DV) proteins and antigens that increase, stimulate, enhance, promote, augment or induce a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c⁺ cells), an anti-Dengue virus (DV) CD4⁺ or CD8⁺ T cell response, production of a Th1 cytokine, a T cell mediated immune response, such as activation of CD8+ T cells, or induction of CD8+ memory T cells, etc.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

Uses and methods of the invention also include, among other things, uses and methods that result in a reduced need or use of another compound, agent, drug, therapeutic regimen, treatment protocol, process, or remedy. For example, for a treatment of Dengue virus (DV) infection or pathology, or vaccination or immunization, a use or method of the invention has a therapeutic benefit if in a given subject a less frequent or reduced dose or elimination of an anti-Dengue virus (DV) treatment results. Thus, in accordance with the invention, uses and methods of reducing need or use of a treatment or therapy for a Dengue virus (DV) infection or pathology, or vaccination or immunization, are provided.

In invention uses and methods in which there is a desired outcome, such as a therapeutic or prophylactic method that provides a benefit from treatment, vaccination or immunization, a Dengue virus (DV) protein, subsequence, portion or modification thereof can be administered in a sufficient or effective amount.

As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by Dengue virus (DV) protein, subsequence, portion or modification thereof alone, optionally in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject. For example, to increase, enhance, improve or optimize immunization and/or vaccination, after an initial or primary administration of one or more Dengue virus (DV) proteins, subsequences, portions or modifications thereof to a subject, the subject can be administered one or more additional “boosters” of one or more Dengue virus (DV) proteins, subsequences, portions or modifications thereof. Such subsequent “booster” administrations can be of the same or a different formulation, dose or concentration, route, etc.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to a use or method of the invention, such as immunization, vaccination and therapeutic treatments.

The term “subject” refers to a subject at risk of DENV exposure or infection as well as a subject that has been exposed or already infected with DENV. Such subjects, include mammalian animals (mammals), such as a non human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans.

Subjects include animal disease models, for example, mouse and other animal models of pathogen (e.g., DENV) infection known in the art.

Accordingly, subjects appropriate for treatment include those having or at risk of exposure to Dengue virus infection or pathology, also referred to as subjects in need of treatment. Subjects in need of treatment therefore include subjects that have been exposed to or contacted with Dengue virus (DV), or that have an ongoing infection or have developed one or more adverse symptoms caused by or associated with Dengue virus (DV) infection or pathology, regardless of the type, timing or degree of onset, progression, severity, frequency, duration of the symptoms.

Target subjects and subjects in need of treatment also include those at risk of Dengue virus (DV) exposure, contact, infection or pathology or at risk of having or developing a Dengue virus (DV) infection or pathology. The invention uses, methods and compositions are therefore applicable to treating a subject who is at risk of Dengue virus (DV) exposure, contact, infection or pathology, but has not yet been exposed to or contacted with Dengue virus (DV). Prophylactic uses and methods are therefore included. Target subjects for prophylaxis can be at increased risk (probability or susceptibility) of exposure, contact, infection or pathology, as set forth herein. Such subjects are considered in need of treatment due to being at risk.

Subjects for prophylaxis need not be at increased risk but may be from the general population in which it is desired to vaccinate or immunize a subject against a Dengue virus (DV) infection, for example. Such a subject that is desired to be vaccinated or immunized against a Dengue virus (DV) can be administered Dengue virus (DV) protein, subsequence, portion or modification thereof. In another non-limiting example, a subject that is not specifically at risk of exposure to or contact with a Dengue virus (DV), but nevertheless desires protect against infection or pathology, can be administered a Dengue virus (DV) protein, subsequence, portion or modification thereof. Such subjects are also considered in need of treatment.

At risk subjects appropriate for treatment also include subjects exposed to environments in which subjects are at risk of a Dengue virus (DV) infection due to mosquitos. Subjects appropriate for treatment therefore include human subjects exposed to mosquitos, or travelling to geographical regions or countries in which Dengue virus (DV) is known to infect subjects, for example, an individual who risks exposure due to the presence of DENV in a particular geographical region or country or population, or transmission from mosquitos present in the region or country. At risk subjects appropriate for treatment also include subjects where the risk of Dengue virus (DV) infection or pathology is increased due to changes in infectivity or the type of region of Dengue virus (DV) carrying mosquitos. Such subjects are also considered in need of treatment due to such a risk.

“Prophylaxis” and grammatical variations thereof mean a use or a method in which contact, administration or in vivo delivery to a subject is prior to contact with or exposure to DENV or DENV infection. In certain situations it may not be known that a subject has been contacted with or exposed to Dengue virus (DV), but administration or in vivo delivery to a subject can be performed prior to infection or manifestation of pathology (or an associated adverse symptom, condition, complication, etc. caused by or associated with a Dengue virus (DV)). For example, a subject can be immunized or vaccinated with a Dengue virus (DV) protein, subsequence, portion or modification thereof. In such case, a use or method can eliminate, prevent, inhibit, suppress, limit, decrease or reduce the probability of or susceptibility towards a Dengue virus (DV) infection or pathology, or an adverse symptom, condition or complication associated with or caused by or associated with a Dengue virus (DV) infection or pathology.

“Prophylaxis” can also refer to a use or a method in which contact, administration or in vivo delivery to a subject is prior to a secondary or subsequent exposure or infection. In such a situation, a subject may have had a prior DENV infection, or have been contacted with or exposed to Dengue virus (DV). In such subjects, an acute DENV infection may but not need be resolved. Such a subject typically has developed anti-DENV antibodies due to the prior exposure or infection. Immunization or vaccination, by administration or in vivo delivery to such a subject, can be performed prior to a secondary or subsequent DENV infection or exposure. Such a use or method can eliminate, prevent, inhibit, suppress, limit, decrease or reduce the probability of or susceptibility towards a secondary or subsequent Dengue virus (DV) infection or pathology, or an adverse symptom, condition or complication associated with or caused by or associated with a Dengue virus (DV) infection or pathology, or an adverse symptom or pathology associated with the development of anti-DENV antibodies, such as ADE.

Treatment of an infection can be at any time during the infection. Dengue virus (DV) protein, subsequence or portion or modification thereof can be administered as a combination (e.g., with a second active), or separately concurrently or in sequence (sequentially) in accordance with the uses and methods as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by Dengue virus (DV) infection, pathology, or an adverse symptom, condition or complication associated with or caused by a Dengue virus (DV). Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Uses and methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

Doses can be based upon current existing protocols, empirically determined, using animal disease models or optionally in human clinical trials. Initial study doses can be based upon animal studies set forth herein, for a mouse, which weighs about 30 grams, and the amount of Dengue virus (DV) protein, subsequence, portion or modification thereof administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg,100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has been previously exposed to, infected with our suffered from Dengue virus (DV), the onset, progression, severity, frequency, duration probability of or susceptibility of the symptom, condition, pathology or complication, or vaccination or immunization to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for treatment, Dengue virus (DV) protein, subsequence, portion or modification thereof will be administered as soon as practical, typically within 1-2, 2-4, 4-12, 12-24 or 24-72 hours after a subject is exposed to or contacted with a Dengue virus (DV), or within 1-2, 2-4, 4-12, 12-24 or 24-48 hours after onset or development of one or more adverse symptoms, conditions, pathologies, complications, etc., associated with or caused by a Dengue virus (DV) infection or pathology. For prophylactic treatment in connection with vaccination or immunization, Dengue virus (DV) protein, subsequence, portion or modification thereof can be administered for a duration of 0-4 weeks, e.g., 2-3 weeks, prior to exposure to, contact or infection with Dengue virus (DV), or at least within 1-2, 2-4, 4-12, 12-24, 24-48 or 48-72 hours prior to exposure to, contact or infection with Dengue virus (DV). For an acute infection, Dengue virus (DV) protein, subsequence, portion or modification thereof is administered at any appropriate time.

The dose amount, number, frequency or duration may be proportionally increased or reduced, as indicated by the status of the subject. For example, whether the subject has a pathogen infection, whether the subject has been exposed to, contacted or infected with pathogen or is merely at risk of pathogen contact, exposure or infection, whether the subject is a candidate for or will be vaccinated or immunized. The dose amount, number, frequency or duration may be proportionally increased or reduced, as indicated by any adverse side effects, complications or other risk factors of the treatment or therapy.

In the uses and methods of the invention, the route, dose, number and frequency of administrations, treatments, immunizations or vaccinations, and timing/intervals between treatment, immunization and vaccination, and viral challenge can be modified. Although rapid induction of immune responses is desired for developing protective emergency vaccines against DENV, in certain embodiments, a desirable DENV vaccine will elicit robust, long-lasting immunity. Thus, in certain embodiments, invention uses, methods and compositions provide long-lasting immunity to DENV. Immunization strategies provided can provide long-lived protection against DENV challenge, depending on the level of vaccine-induced CD8+ T cell response.

The invention also provides an amount of a Dengue virus protein, subsequence or portion, or modification thereof for use in: eliciting, stimulating, inducing, promoting, increasing, or enhancing an anti-Dengue virus T cell response in a subject without eliciting or sensitizing the subject to severe dengue disease (e.g., ADE mediated DHF or DSS) upon a secondary or subsequent Dengue virus infection; providing a subject with protection against a Dengue virus infection or pathology, or one or more physiological disorders, illness, diseases or symptoms caused by or associated with Dengue virus infection or pathology without eliciting or sensitizing the subject to severe dengue disease (e.g., ADE mediated DHF or DSS) upon a secondary or subsequent Dengue virus infection; vaccinating a subject against a Dengue virus infection without eliciting or sensitizing the subject to severe dengue disease (e.g., ADE mediated DHF or DSS) upon a secondary or subsequent Dengue virus infection; and treating a subject for a Dengue virus infection without eliciting or sensitizing the subject to severe dengue disease (e.g., ADE mediated DHF or DSS) upon a secondary or subsequent Dengue virus infection. In certain embodiments, DENV proteins, subsequences, portions and modifications thereof may be pharmaceutical compositions.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

To increase an immune response, immunization or vaccination, Dengue virus (DV) proteins, subsequences, portions and modifications thereof can be coupled to another protein such as ovalbumin or keyhole limpet hemocyanin (KLH), thyroglobulin or a toxin such as tetanus or cholera toxin. Dengue virus (DV) proteins, subsequences, portions and modifications thereof can also be mixed with adjuvants.

Adjuvants include, for example: Oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria Molina tree, see, e.g., “Saponin adjuvants”, Archiv. fur die gesamte Virusforschung, Vol. 44, Springer Verlag, Berlin, p243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

Cosolvents may be added to a Dengue virus (DV) protein, subsequence, portion or modification composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel ad Soklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Dengue virus (DV) proteins, subsequences, portions, and modifications thereof, along with any adjunct agent, compound drug, composition, whether active or inactive, etc., can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to a “Dengue virus (DV) protein, subsequence, portion, or modification thereof,” or a “Dengue virus (DV)” includes a plurality of Dengue virus (DV) proteins, subsequences, portions, and modifications thereof, such as CD4⁺ and/or CD8⁺ T cell epitopes, or serotypes of Dengue virus (DV), and reference to an “activity or function” can include reference to one or more activities or functions of a Dengue virus (DV) protein, subsequence, portion, or modification thereof, including function as a T cell epitopes, an ability to elicit, stimulate, induce, promote, increase, enhance or activate a measurable or detectable anti-DV CD4⁺ T cell response or anti-DV CD8⁺ T cell response, and so forth.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. For example, in certain embodiments or aspects of the invention, antibodies or other materials and method steps are excluded. In certain embodiments and aspects of the invention, for example, a Dengue virus (DV) protein, subsequence, portion, or modification thereof, is excluded. Thus, even though the invention is generally not expressed herein in terms of what is not included, embodiments and aspects that expressly exclude compositions or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of an ADE mouse model that reflects ADE in humans.

Antibody (Ab)-induced dengue disease is a severe condition that affects humans having existing Dengue virus antibodies. A clinically relevant model of antibody (Ab)-induced dengue disease (ADE) in mice is disclosed. The model demonstrates, for the first time, ADE in vivo (Zellweger, et al. Cell Host Microbe 7:128-139 (2010)).

Briefly, AG129 mice were passively administered 15 μg of mouse mAb of subclass IgG2a (clone 2H2; DENV1-4 cross-reactive) before infection with 5×10⁸ genomic equivalents (GE) (≈10⁴ PFU) of the DENV2 strain S221. Mice treated with 2H2 succumbed early to S221 infection (day 4-6) and featured the hallmarks of severe dengue disease in humans (high viral load, elevated hematocrit, cytokine storm, low platelet count, increased vascular permeability, hemorrhagic manifestations, and shock-induced death). In contrast, mice treated with isotype control Ab developed paralysis at later times after infection (day 10-30).

### Example 2

This example includes data demonstrating that vaccination with inactivated Dengue virus mediates ADE.

The demonstration of ADE in the clinically relevant animal model of human ADE allows the evaluation of aspects of protective versus pathogenic effects of dengue vaccination. Three general types of dengue vaccines are currently under development, inactivated, subviral particles or subunit, and live attenuated (Murphy, et al. Ann. Rev. of Immunol. 29:587-619 (2011)). First assessed was whether UV-inactivated DENV2 in alum can mediate protection as an inactivated vaccine candidate. Alum was chosen as the adjuvant because it is used in many human vaccines and is known to promote humoral immunity, which is believed to be required for dengue vaccine-mediated protection.

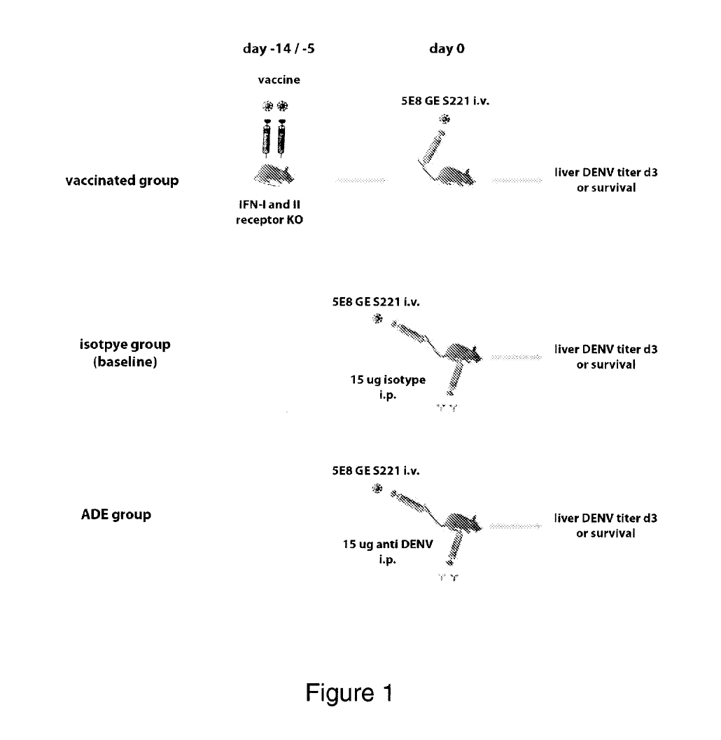

AG129 mice were injected with 10¹¹ GE (≈2×10⁶ PFU) of UV-inactivated DENV2 strain S221 via a subcutaneous (s.c.) or intraperitoneal (i.p.) route 14 and 5 days before a sublethal intravenous (i.v.) infection with S221 (5 ×10⁸ GE or ≈104 PFU) (schematized in FIG. 1). Control groups included a baseline/isotype group (i.p. injected with 15 μg of an irrelevant isotype control Ab prior to viral challenge) and an ADE group (i.p. injected with 15 μg of DENV prM/M-specific IgG2a mAb clone 2H2 prior to viral challenge).

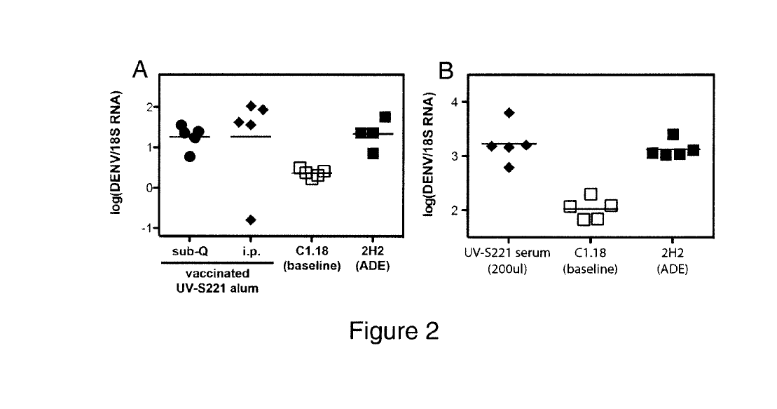

DENV RNA levels in the liver at day 3 after viral challenge were measured by qRT-PCR analysis. As expected, control mice with enhancing Ab (i.e. the ADE group) contained ≈10 fold-higher viral RNA levels than the baseline/isotype group (FIG. 2A). Similarly to the ADE group, both the s.c. and i.p. groups of vaccinated animals contained high viral RNA levels (FIG. 2A), and most of the vaccinated animals died between days 4-5 post-infection, thereby demonstrating ADE effect upon immunization with UV-inactivated DENV2 in alum.

To confirm that antibodies were responsible for the high viral load in the liver of UV-inactivated DENV2 in alum-vaccinated mice, serum from immunized mice was passively transferred i.v. into naïve mice 1 day before viral challenge. Mice administered the immunized mouse serum had elevated levels of DENV RNA in the liver at day 3 post-challenge (FIG. 2B), in agreement with the results shown in FIG. 2A. These data demonstrate that the UV-inactivated DENV2 alum immunization strategy induces ADE instead of protection in mice. Without being limited to or bound by any particular theory, it may be that the failure of UV-inactivated DENV2 alum immunization to elicit a sufficient T-cell response resulted in lack of protection against DENV, and instead resulted in inducing the occurrence of ADE.

### Example 3

This example includes data demonstrating that Dengue Virus protein can provide protective immunity, without substantially inducing ADE, and even in the presence of enhancing antibodies.

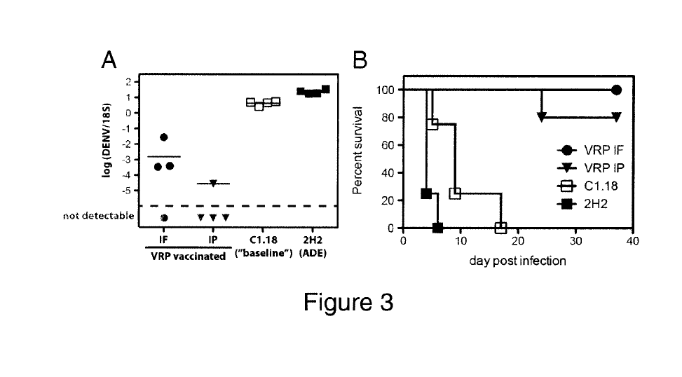

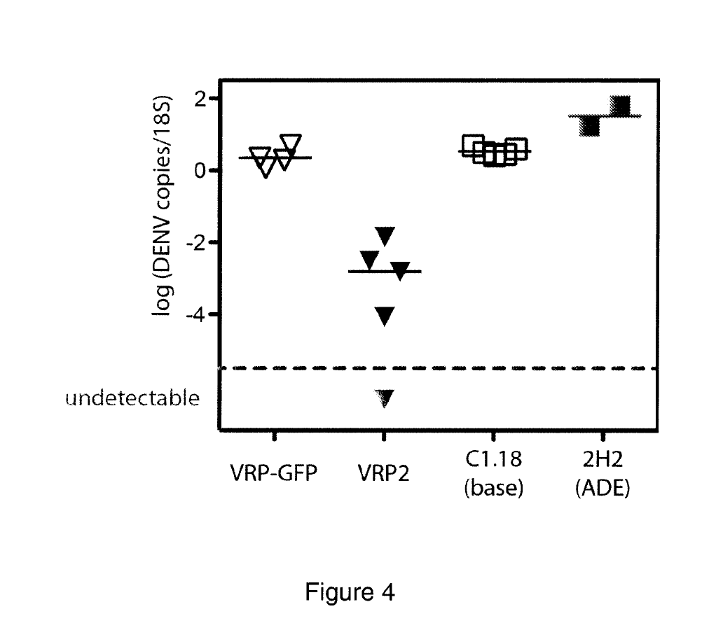

To ascertain the ability of a DENV2 envelope (E) protein to provide protection against Dengue virus, non-propagating Venezuelan Equine Encephalitis (VEE) virus replicon particles (VRP) coding for DENV2 envelope (E) protein (i.e. VRP-DENV2E) were used. UV-inactivated DENV2 plus alum regimen is shown in FIG. 1. In brief, AG129 mice were immunized i.p. or intra-foot pad (i.f.) with 10⁶ GE of VRP-DENV2E (White, et al. Journal of Virol. 81:10329-10339 (2007)) on 14 and 5 days prior to challenge with the sub-lethal dose of S221. All mice vaccinated with DENV2E had lower viral RNA levels than even the baseline/isotype group in the liver at day 3 post-challenge (FIG. 3A). As expected, most ADE mice developed the early lethal disease between days 3-5 post-challenge and most baseline/isotype mice exhibited paralysis between day 7-14 post-challenge. In contrast, the majority of mice in both the i.f. and i.p. DENV2E vaccinated groups survived the challenge and failed to develop even paralysis (FIG. 3B). Using VRP-GFP (which codes for the irrelevant GFP protein) instead of DENV2E did not reduce liver viral load 3 days after challenge, thereby confirming the specificity of DENV2E-mediated protective immunity (FIG. 4). Collectively, these results indicate that the immunization strategy using DENV2E confers protection in mice upon challenge with DENV2.

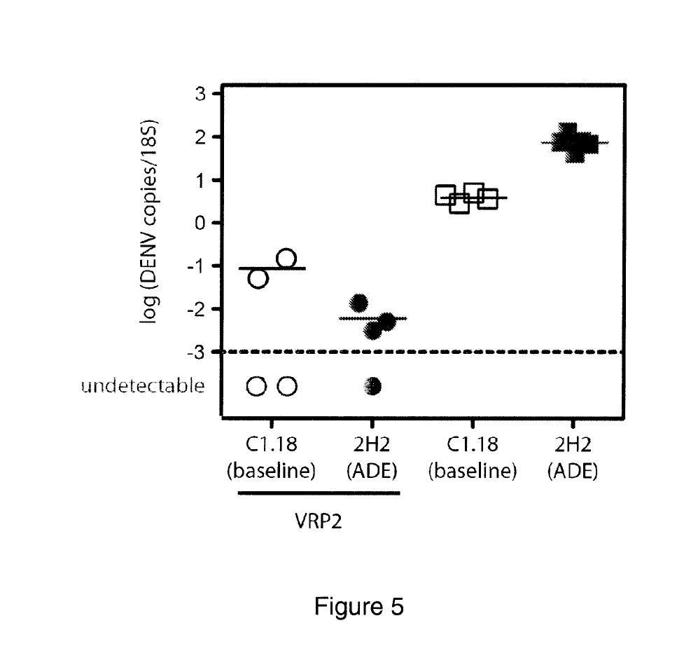

To explore the nature of DENV2E-mediated protection of mice, it was determined whether vaccination would provide protective immunity upon ADE challenge. AG129 mice were immunized with VRP-DENV2E on 14 and 5 days before viral challenge (i.e. the same immunization protocol as all studies described thus far), but the immunized mice were administered anti-DENV mAb (15 μg of clone 2H2) just prior to i.v. inoculation with S221. It was found that DENV2E-vaccination reduced viral RNA levels in the liver on day 3 after challenge with virus alone or with virus plus anti-DENV Ab, indicating that DENV2 immunization strategy offers protection even in the presence of enhancing Abs (FIG. 5).

In the animal studies described in White, et al., supra the animals do not and are not capable of developing ADE. Thus, in contrast to the model disclosed herein which develops ADE, the animal model in White, et al., supra does not reflect human DENV infection, particularly humans previously infected with or exposed to DENV that have developed anti-DENV antibodies and are therefore at risk of ADE upon subsequent infection or exposure to DENV. Furthermore, the studies in White, et al. are limited to analysis of anti-DENV antibodies that purportedly provide protection, but as disclosed herein antibodies exacerbate Dengue virus illness upon a secondary or subsequent DENV infection or exposure of an individual who has developed such anti-DENV antibodies, resulting in ADE. Moreover, subsequent studies indicated that tetravalent immunization with all four Dengue virus serotypes is required to produce a broad spectrum antibody response, which antibodies were merely shown to be capable of neutralizing Dengue virus in vitro, but not broad spectrum protection against two or more DENV serotypes, from infection and/or symptoms associated with or caused by DENV infection, and do not demonstrate a lack of producing substantial ADE, or eliciting, inducing or promoting ADE, since such studies are in animals that do not develop ADE and therefore are not reflective of DENV infection in humans, particularly those that have developed antibodies to one or more DENV serotypes and are therefore at risk of ADE.

### Example 4

This example includes data demonstrating that cell-mediated immunity contributes to the DENV2E-mediated protection against DENV.

To analyze the mechanisms by which the DENV2E vaccination provides protective immunity, antibody responses induced by the two qualitatively different vaccine candidates (UV-inactivated 5221 plus alum compared to VRP-DENV2E) were first compared. One day before viral challenge, serum samples were collected from the immunized mice that were used for studies in FIGS. 2 and 4. DENV-specific serum IgG was measured by ELISA on sucrose gradient-purified S221 virions coated plates, and the neutralization capacity of serum was determined using flow cytometry-based neutralization assay with C6/36 mosquito cells (White, et al., supra).

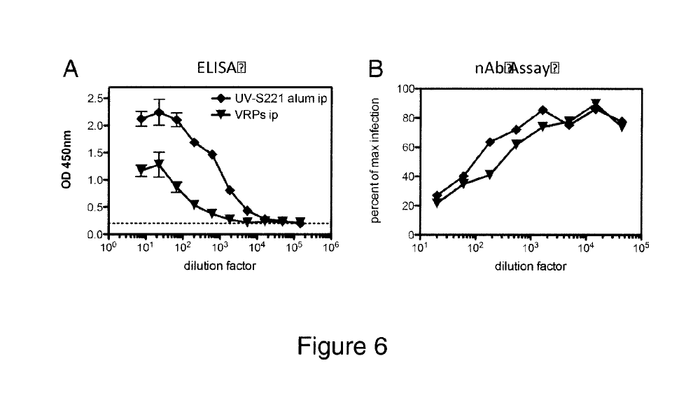

Although direct comparison of DENV-specific IgG levels between the two groups of immunized mice is not feasible due to the presence of different antigens in UV-inactivated virus (which contains both prM/M and E protein) versus DENV2E (which contains only E), both vaccine candidates induced DENV-specific binding Abs in all immunized mice (FIG. 6A). Despite the detection of higher DENV-specific IgG levels in mice immunized with UV-inactivated S221 plus alum than those immunized with VRP-DENV2E, neutralizing-antibody titers appear to be similar between the 2 groups of immunized mice (FIG. 6B). This result indicates that cell-mediated, rather than humoral immunity, contributes to the DENV2E-mediated protection of mice against DENV.

### Example 5

This example includes data demonstrating that CD8+ T cells provide early protective capacity against Dengue virus.

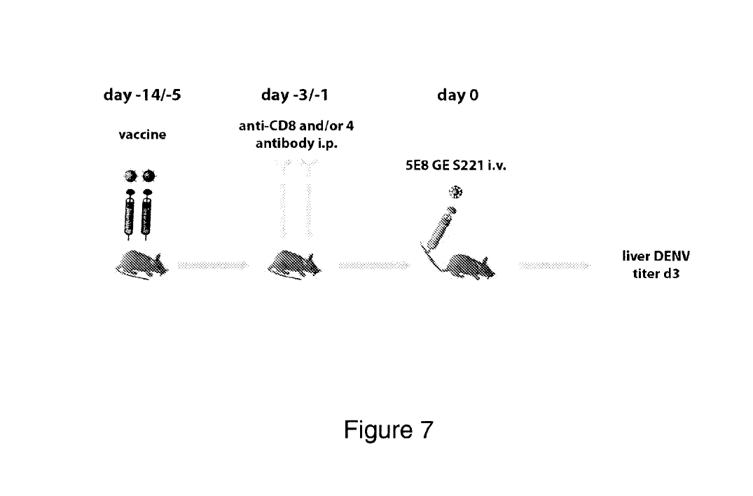

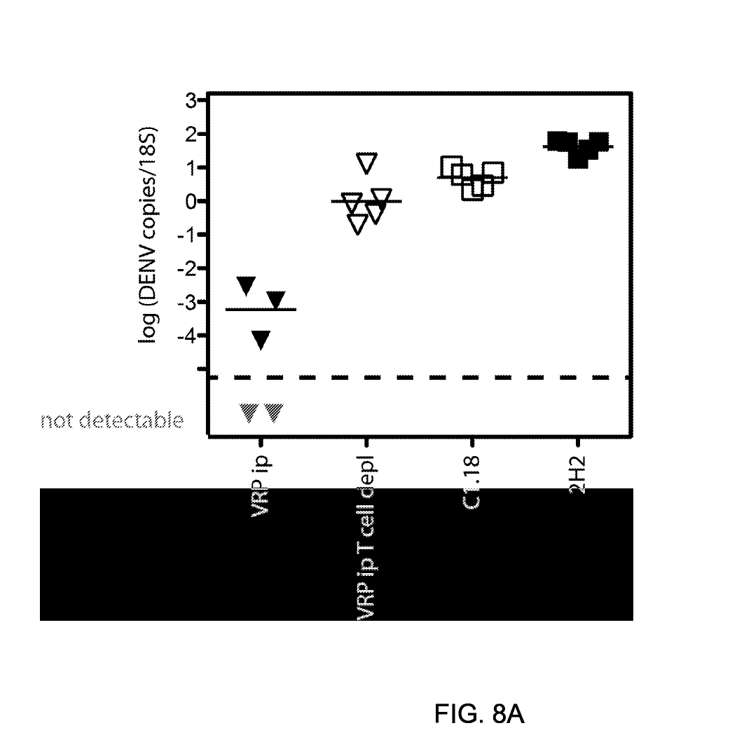

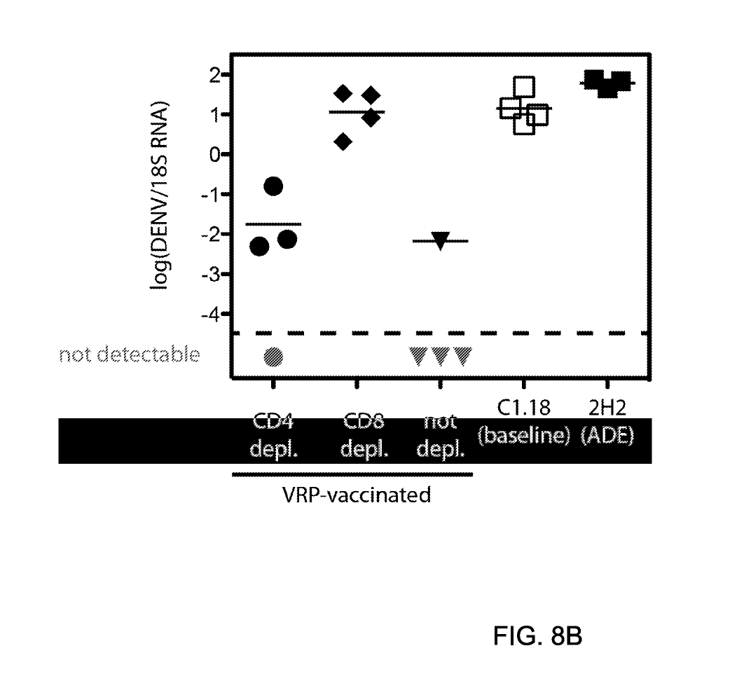

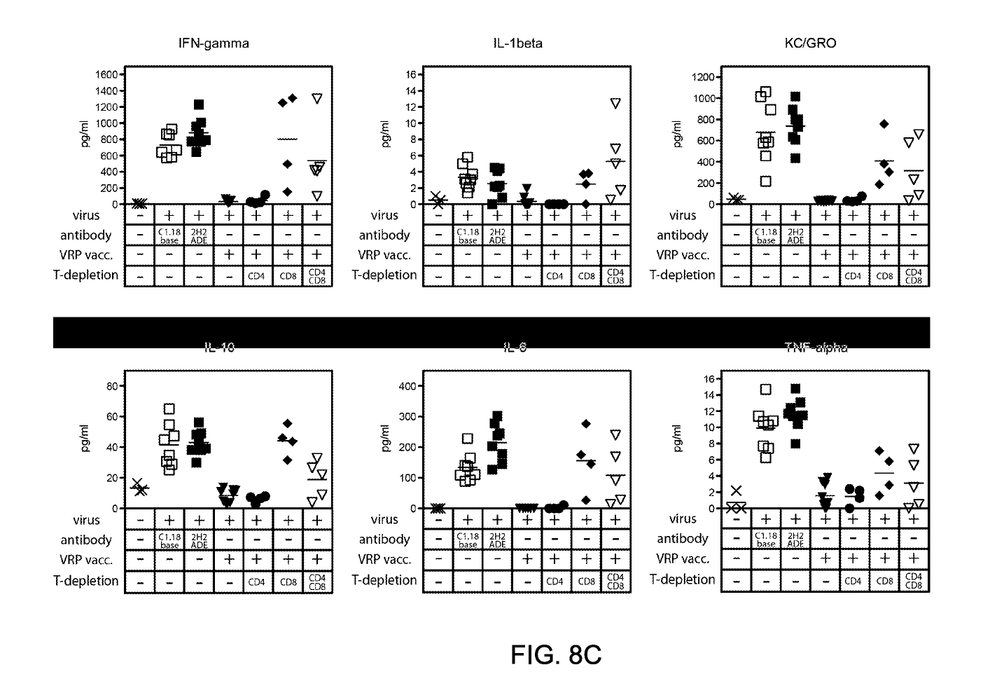

To measure the contribution of T cells in DENV2E vaccine-mediated protection, mice were immunized with VRP-DENV2E as described above, followed by depletion of CD4+ and/or CD8+ T cells before challenge with S221 (FIG. 7). On day 3 post-challenge, viral RNA levels in liver and cytokine levels in the serum were measured (FIG. 8). Depletion of both CD4+ and CD8+ T cells from immunized animals abolished protection (FIG. 8A), whereas depletion of CD4+ T cells alone had little to no effect on DENV viral load, compared to immunized but non-depleted mice (FIG. 8B). Consistent with these viral load data, immunized mice that were depleted of both CD4+ and CD8+ T cells or only CD8+ T cells contained elevated levels of serum cytokines as compared with undepleted and CD4+ T cell-depleted immunized mice (FIG. 8C). Collectively, these results demonstrate that CD8+ T cells are required for controlling DENV viral load and cytokine storm upon DENV challenge of the immunized animals, thereby revealing an essential role of CD8+ T cells in providing early protective capacity conferred by the DENV2E immunization strategy.

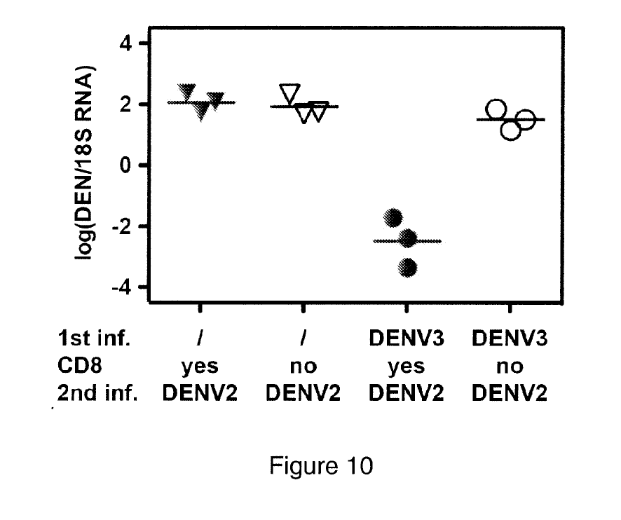

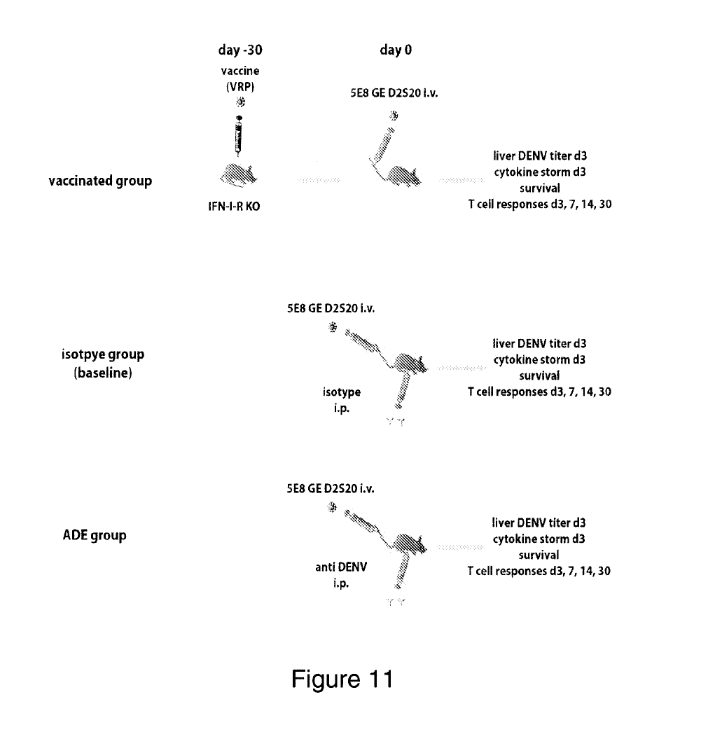

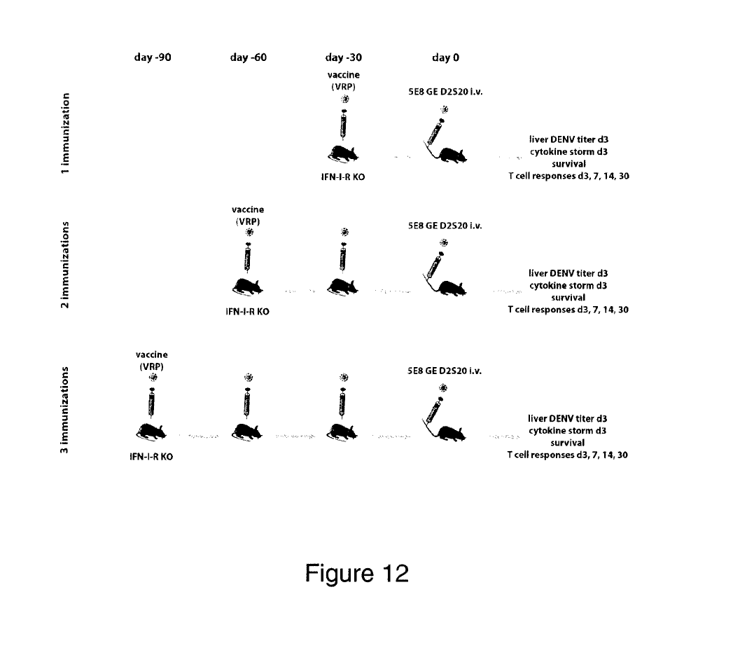

Studies examining heterologous DENV infections were also conducted. Following infection with live DENV3 (UNC3001), CD8+ T cells were depleted in mice by administration of an anti-CD8+ antibody, as discussed herein. The mice were then infected with live DENV 2 (S221). DENV2 viral RNA levels in the liver of mice were determined by qRT-PCR (FIG. 10). DENV2 viral RNA levels were elevated in the absence of CD8+ T cells, whereas in the presence of CD8+ T cells protection against DENV2 was observed. This data demonstrates that CD8+ T cells are effective at protection against heterologous DENV infection.

### Example 6

This Example includes studies demonstrating that adoptively transferred wild-type T cells protect against DENV in AG129 mice.

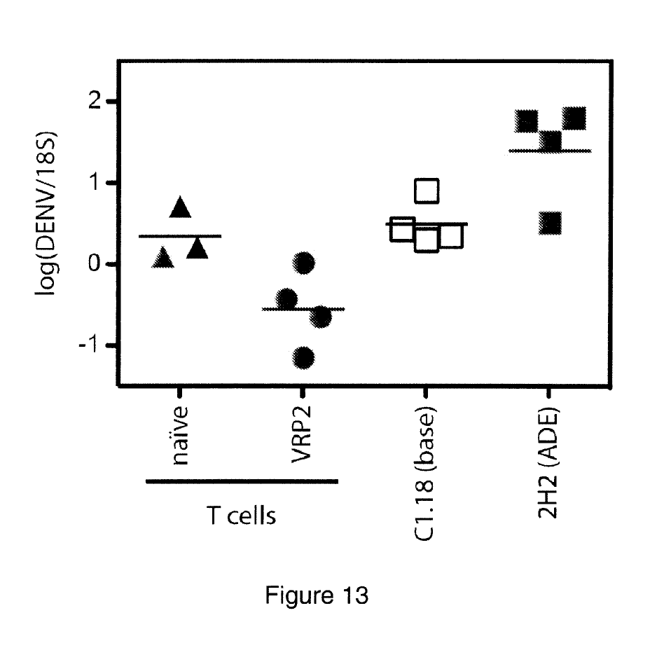

Wild-type 129/Sv mice were immunized i.p. with 10⁶ GE of VRP-DENV2E on days −14 and −5, followed by isolation of total T cells (both CD4+ and CD8+) by MACS negative selection on day 0 and i.v. transfer into AG129 mice (FIG. 13). One day after T cell transfer, AG129 mice were challenged with 5×10⁸ GE of S221 i.v. The control groups represent non-immunized AG129 mice that were treated i.p. with 15 μg of 2H2 (ADE, black squares) or C1.18 (baseline, white squares) 1 hour before viral challenge. DENV RNA levels in the liver were measured 72 hours after infection by qRT-PCR. Each symbol represents a mouse. This data also reveals that T-cells are involved in protection against DENV.

### Example 7

As disclosed herein, the data indicate a rapidly protective, CD8+ T cell-dependent DENV immunization strategy using DENV2E in a clinically relevant model of DENV infection. Although fast induction of immune responses is important for developing protective emergency vaccines against DENV, a desirable dengue vaccine should elicit robust, long-lasting immunity. Accordingly, length of protection and uses and methods to augment magnitude and duration of CD8+ T cell immunity, if such augmentation is desired, can be obtained by adjusting one or more of the following parameters.

It is widely acknowledged that multiple dosing or higher dosing with replication-incompetent attenuated viruses can induce T cells responses that are comparable to those induced by replication-competent virulent poxviruses (Earl, et al. Nature 428:182-185 (2004); Peters, et al. Vaccine 25:2120-2127 (2007). Based on these observations, in the invention, the route, dose, number of immunizations can be increased, and intervals between immunization optimized.

In general, activated CD8+ T cells are CD44^{hi} CD62L^{low} Ki-67+Bcl-2low; effector CD8+ T cells are CD107a⁺ granzyme B⁺ perforin⁺; short-lived effector cells (SLECs) are KLRG1⁺CD127⁺, memory precursor effector cells (MPECs) are KLRG1⁻CD127⁺; central memory T (TCM) are CD62L⁺CD127^{±}; and effector memory T (TEM) are CD62L⁻CD127⁺. It is expected that the highest DENV2E dose (translating to a greater antigenic load over time) and s.c. route (likely leading to the induction of T_{RM} in the skin and perhaps liver in addition to T_{CM} cells) will induce more memory CD8+ T cells than lower doses of DENV2E by way of i.p. adminstration—the greater CD8+ T cell response should respond faster upon viral challenge and correlate with better protection (i.e. the immunized mice should have increased survival and decreased levels of viral RNA in the liver and cytokines in the serum upon viral challenge).

Days between immunization can be optimized, for example, if 30 days between immunizations is too short due to delayed T cell contraction upon repeated immunizations, longer intervals between immunizations, such as 45, 60, or 90 days can be employed.

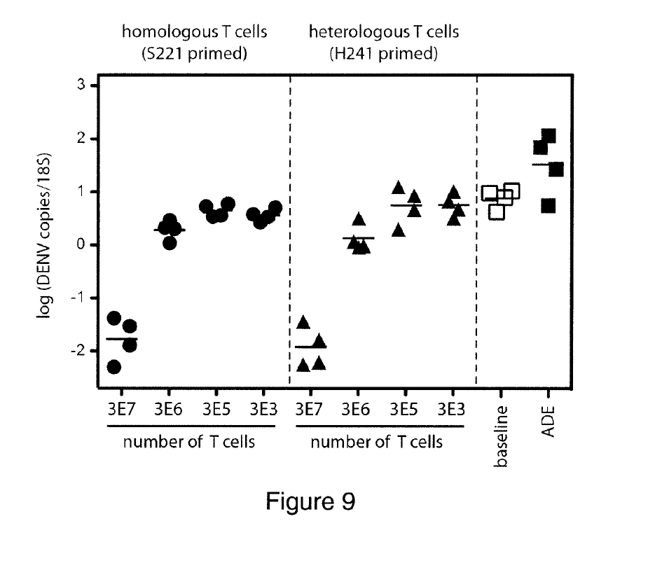

Finally, the data disclosed herein show that CD4+ T cells are not necessary for CD8+ T cell-dependent protection provided by DENV2E immunization (FIG. 9). Based on these observations and without being limited to or bound by any particular theory, it appears that CD4+ T cells may not be required for recall immunity mediated by DENV2E-elicited CD8+ T cells. Accordingly, the level of CD8+ T cell response should correlate with protection against DENV.

### Example 8

This example includes a description of various materials and methods.

### Mice and Infections

C57BL/6 (H-2^{b}) mice were obtained from The Jackson Laboratory and subsequently bred. IFN-α/βR^{-/-} mice on the C57BL/6 background were obtained from Dr. Wayne Yokoyama (Washington University, St. Louis, Mo.) via Dr. Carl Ware. HLA-A*0201/Kb, A*1101/Kb, A*0101, B*0702 and DRB1*0101 transgenic mice were bred at LIAI as previously described (Kotturi et al., Immunome Res 6:4 (2010); Pasquetto et al., J Immunol 175:5504 (2005); Alexander et al., J Immunol 159:4753 (1997); Alexander et al., Hum Immunol 64:211 (2003)). All transgenic mouse strains were subsequently backcrossed with the IFN-α/βR^{-/-} mice at the animal facility at LIAI.B6.SJL mice were purchased from Taconic. Mice were used between 5 and 10 weeks of age.

Mice were infected intravenously (i.v.) in the lateral tail vein or retro-orbitally (r.o.) with 200 μl of the DENV2 strain, S221, in 5% FBS/PBS. Blood was obtained from anesthetized mice by r.o. puncture. For studies with transgenic mice, mice were infected i.v.r.o. with 10¹⁰ genomic equivalents (GE) of S221 in 100 uL PBS. On day 7 post-infection, mice were sacrificed and splenic CD8+ or CD4+ T cells, respectively, were used in mouse IFNγ ELISPOT assays. All mouse studies were approved by the Animal Care Committee.

### Cell Culture and Viral Stocks

The hybridoma clones SFR3, GK1.5, and 2.43, which produce rat anti-human HLA-DRS, anti-mouse CD4, and anti-mouse CD8 IgG2b Ab, respectively, were from the American Type Culture Collection, and were grown in Protein-Free Hybridoma Medium supplemented with penicillin, streptomycin, HEPES, G1utaMAX, and 2-ME (all from Invitrogen) at 37° C., 5% CO₂. C6/36, an A. albopictus mosquito cell line, was cultured in Leibovitz's L-15 Medium (Invitrogen) supplemented with 10% FBS (Gemini Bio-Products), penicillin, streptomycin, and HEPES at 28° C. in the absence of CO₂. S221, a plaque-purified DENV2 strain, was derived from the clinical isolate, PL046 (Lin et al., J Virol 72:9729 (1998)), as described previously (Yauch et al., J Immunol 182:4865 (2009)). Viral stocks were amplified in C6/36 cells and purified over a sucrose gradient as previously described (Prestwood et al., J Virol 82:8411 (2008)). Infectious doses were determined based on GE, which were quantified by real-time RT-PCR. There are approximately 5×10⁴ GE/PFU for S221, based on plaque assay on baby hamster kidney cells.

### Bioinformatic Analyses

Candidate epitopes were identified using a consensus approach (Wang et al., PLoS Comput Biol 4:e1000048 (2008)). Briefly, all 15-mer peptides that are encoded in the DENV2 PL046 polyprotein were predicted for binding to H-2 I-A^{b}. Two independent algorithms (Zhang et al., Nucleic Acids Res 36:W513 (2008)) were used to rank the peptides by predicted binding affinity. The median of the two ranks was used to select the top 73 out of 3383 peptides, corresponding to the top 2% of all peptides.

For human MHC class I binding predictions all 9 and 10 mer peptides were predicted for their binding affinity to their respective alleles. Binding predictions were performed using the command-line version of the consensus prediction tool available on the IEDB web site (Zhang et al., Nucleic Acids Res 36:W513 (2008)). Peptides were selected if they are in the top 1% of binders in a given strain. For human MHC class II binding predictions all 15 mer peptides were predicted for their binding affinity to the DRB1*0101 allele. As with class I, binding predictions were performed using the command-line version of the consensus prediction tool available on the IEDB web site. The top 2% of predicted binders were then selected for synthesis. All peptides evaluated in this study were derived from the DENV2 virus strain S221, which was also used as infectious agent in this study, as described above. For the conservancy analysis, full-length DENV polyprotein sequences were retrieved for each serotype from the NCBI Protein database using the following query: txid11053 AND polyprotein AND 3000:5000[slen]. The number of isolates from any one country was limited to 10 to eliminate geographical bias. Sequences were considered “unique” if they varied by at least 1 amino acid from all other sequences. In summary, 171 DENV2, 162 DENV1, 169 DENV3 and 53 DENV4 sequences from the NCBI protein database were investigated for conservancy of the identified epitopes within the respective serotypes.

### Peptide Synthesis

Peptides utilized in initial screening studies were synthesized as crude material by A and A Labs. A total of 73 15-mer peptides were ordered and synthesized twice in different (alphabetical vs. predicted IC₅₀) order. Positive peptides were re-synthesized by A and A Labs and purified to >90% homogeneity by reverse-phase HPLC. Purity of these peptides was determined using mass spectrometry. The HPLC-purified peptides were used for all subsequent studies.

All peptides using human MHC class I or II sequences were synthesized by Mimotopes (Victoria, Australia). MHC class I predictions led to the synthesis of a total of 431 9-mer and 10-mer peptides. Peptides were made as crude material and combined into pools of 10 individual peptides, according to their predicted HLA restriction. MHC class II predictions resulted in the synthesis of 12 15-mers, which were tested individually.

### Flow Cytometric Analyses

For surface staining of germinal center B cells, splenocytes were stained with anti-B220Alexa Fluor 647 (Biolegend), anti-CD4-PerCP (BD Biosciences), GL7-FITC (BD Biosciences), anti-IgD-eFluor 450 (eBioscience), and anti-Fas-PE (BD Biosciences). For intracellular cytokine staining (ICS) of CD4⁺ T cells, 2×10⁶splenocytes were plated in 96-well U-bottom plates and stimulated with individual DENV2 peptides (3 μg/ml) for 2 h (hours). Brefeldin A (GolgiPlug, BD Biosciences) was then added and cells were incubated for another 5 h (hours). Cells were washed, incubated with supernatant from 2.4G2-producing hybridoma cells, and labeled with anti-CD4-eFluor 450 (eBioscience) and anti-CD8α-PerCP-eFluor 710 (eBioscience) or PE-Cy7 (BD Biosciences). The cells were then fixed and permeabilized using the BD Cytofix/Cytoperm Kit, and stained with various combinations of anti-IFN-γ-APC (eBioscience), anti-TNF-PE-Cy7 (BD Biosciences), anti-IL-2-Alexa Fluor 488 (BD Biosciences) or -PE (Biolegend), and anti-CD40L-PE (eBioscience). Foxp3 staining was done using the mouse regulatory T cell staining kit from eBioscience. The criteria for positivity in CD4⁺ T cell epitope identification were: 2× the percentage of IFN-γ produced by stimulated cells compared with unstimulated cells, positive in two independent crude peptide orders, and positive when ordered as HPLC-purified (>90% pure). For CD8⁺ T cell ICS, splenocytes (2×10⁶) were stimulated in 96-well U-bottom plates for 5 h (hours) in the presence of 1 μg/ml H-2^{b}-restricted epitopes identified previously: M₆₀₋₆₇, NS2A₈₋₁₅, and NS4B₉₉₋₁₀₇ (Yauch et al., J Immunol 182:4865 (2009)). Anti-CD107a-FITC (BD Biosciences) was added to the wells during the stimulation. Cells were then stained as described for CD4⁺ T cell ICS. Samples were read on an LSR II (BD Biosciences) and analyzed using FloJo software (Tree Star).

### Immunohistochemistry

Tissues were embedded in O.C.T. compound (Sakura). Sections (6 μm) were cut and stored at −80° C. Frozen sections were thawed and fixed for 10 minutes in acetone at 25° C., followed by 8 minutes in 1% paraformaldehyde (EMS) in 100 mM dibasic sodium phosphate containing 60 mM lysine and 7 mM sodium periodate pH 7.4 at 4° C. Sections were blocked first using the Avidin/Biotin Blocking Kit (Vector Labs) followed by 5% normal goat serum (Invitrogen) and 1% BSA (Sigma) in PBS. Sections were stained overnight with anti-F4/80-biotin (clone BM8, Biolegend), anti-CD4-PE (clone RM4-5, eBioscience), anti-CD8β-Alexa Fluor 647 (clone YTS156.7.7, Biolegend), and anti-B220-FITC (clone RA3-6B2, BD Pharmingen). Sections were then washed and stained with streptavidin-Alexa Fluor 750 and rabbit anti-FITC-Alexa Fluor 488 (Invitrogen). Images were recorded using a Leica TCS SP5 confocal microscope, processed using Leica Microsystems software, stitched together using Adobe Illustrator, and adjusted using ImageJ.

### T Cell Depletions

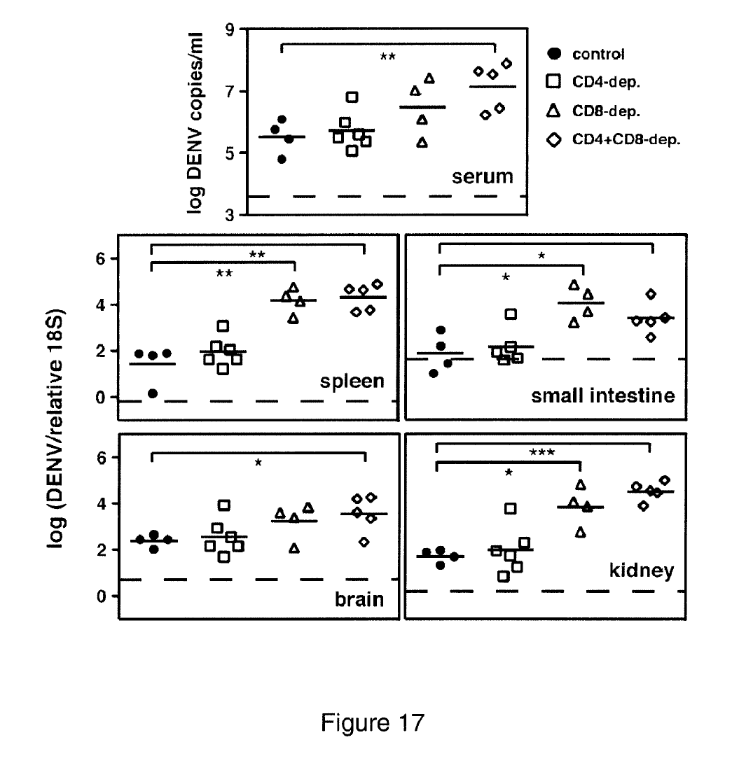

Hybridoma supernatants were clarified by centrifugation, dialyzed against PBS, sterile-filtered, and quantified by BCA Protein Assay Reagent (Thermo Scientific), IFN-α/βR^{-/-} mice were injected i.p. with 250 μg of SFR3, or GK1.5, or 2.43 in PBS (250 μl total volume) 3 days and 1 day before or 1 day before and 1 day after infection, which resulted in depletion of ≧90% of CD8⁺ cells and ≧97% of CD4⁺ cells. In FIG. 17, one CD4-depleted mouse received GK1.5 only on day 1, which still resulted in 97% depletion.

### DENV2-Specific Antibody ELISA

Serum was harvested from control and CD4-depleted IFN-α/βR^{-/-} mice 7 days after infection with 10¹⁰ GE of DENV2, or naïve mice. EIA/RIA 96-well plates (Costar) were coated with DENV2 (10⁹ GE per well) in 50 μl 0.1M NaHCO₃. The virus was UV-inactivated and plates left overnight at 4° C. The plates were then washed to remove unbound virus using 0.05% (v/v) Tween 20 (Sigma) in PBS. After blocking with Blocker Casein Blocking Buffer (Thermo Scientific) for 1 h at room temperature, 1:3 serial dilutions of serum in a total volume of 100 μl were added to the wells. After 1.5 h, wells were washed and bound antibody was detected using HRP-conjugated goat anti-mouse IgG Fc portion or HRP-conjugated donkey anti-mouse IgMμ chain (Jackson Immunoresearch) and TMB (eBioscience).

### Antibody-Virus Neutralization Assay

Serum was heat-inactivated at 56° C. for 30 min. Three-fold serial dilutions of serum were then incubated with 5×10⁸ GE of DENV2 for 1 h at room temperature in a total volume of 100 μl PBS. Next, approximately 6×10⁵ C6/36 cells per well of a 24-well plate were infected with 100 μl of the virus-antibody mix for one hour at 28° C. Cells were washed twice with 500 μl of PBS, and then incubated at 28° C. in 500 μl L-15 Medium containing 5% FBS, penicillin, and streptomycin for 24 h. For each antibody dilution, the percentage of infected cells was determined by flow cytometry as previously described (Lambeth et al., J Clin Microbiol 43:3267 (2005)) using 2H2-biotin (IgG2a anti-prM/M, DENV1-4 reactive) and streptavidin-APC (Biolegend). The percentage of infected cells was normalized to 100% (infection without serum).

### CD8 In Vivo Cytotoxicity Assay

IFN-α/βR^{-/-} mice (recipients) were infected with 10¹⁰ GE of DENV2. Some mice were depleted of CD4⁺ T cells before infection. Splenocytes (targets) were harvested from donor B6.SJL congenic mice (CD45.1) 7 days later. RBC were lysed, and the target cells were pulsed with varying concentrations of a pool of 4 H-2^{b}-restricted DENV2 peptides (M₆₀₋₆₇, NS2A₈₋₁₅, NS4B₉₉₋₁₀₇, NS5₂₃₇₋₂₄₅) or DMSO for 1 h at 37° C. The cells were then washed and labeled with CFSE (Invitrogen) in PBS/0.1% BSA for 10 min at 37° C. Cells were labeled with 1 μM CFSE (CFSE^{high})or 100 nM CFSE (CFSE^{low}) or left unlabeled. After washing, the cell populations were mixed and 5×10⁶ cells from each population were injected i.v.into naïve or infected recipient mice. After 4 h, the mice were sacrificed and splenocytes stained with anti-CD45.1-APC (eBioscience) and analyzed by flow cytometry, gating on CD45.1⁺ cells. The percentage killing was calculated as follows: 100−((percentage DENV peptide-pulsed in infected mice/percentage DMSO-pulsed in infected mice)/(percentage DENV peptide-pulsed in naïve mice/percentage DMSO-pulsed in naïve mice)×100).

### CD4 In Vivo Cytotoxicity Assay

IFN-α/βR^{-/-} mice (recipients) were infected with 10¹⁰ GE of DENV2. Some mice were depleted of CD4⁺ or CD8⁺ cells before infection. Splenocytes (targets) were harvested from donor B6.SJL congenic mice (CD45.1) 7 days later. RBC were lysed and the target cells were pulsed with 1.7 μg (approximately 1 μM) each of NS2B₁₀₈₋₁₂₂, NS3₁₉₈₋₂₁₂, and NS3₂₃₇₋₂₅₁ (or DMSO) for 1 h at 37° C. The cells were then washed and labeled with CFSE in PBS/0.1% BSA for 10 min at 37° C. DENV2 peptide-pulsed cells were labeled with 1 μM CFSE (CFSE^{high}) and DMSO-pulsed cells with 100 nM CFSE (CFSE^{low}). After washing, the two cell populations were mixed and 5×10⁶ cells from each population were injected i.v. into naïve or infected recipient mice. After 16 h, the mice were sacrificed and splenocytes stained and the percentage killing calculated as described for the CD8 in vivo cytotoxicity assay.

### Quantitation of DENV Burden in Mice

Mice were euthanized by isoflurane inhalation and blood was collected via cardiac puncture. Serum was separated from whole blood by centrifugation in serum separator tubes (Starsted). Small intestines were put into PBS, flushed, filleted, chopped into small pieces, and put into RNA later (Qiagen). Other organs were immediately placed into RNAlater and all organs were subsequently homogenized for 3 min in 1 ml tissue lysis buffer (Qiagen Buffer RLT) using a Mini-Beadbeater-8 (BioSpec Products) or QiagenTissueLyser. Immediately after homogenization, all tissues were centrifuged (5 min, 4° C., 16,000×g) to pellet debris, and RNA was isolated using the RNeasy Mini Kit (Qiagen). Serum RNA was isolated using the QIAamp Viral RNA Mini Kit (Qiagen). After elution, viral RNA was stored at −80° C. Quantitative RT-PCR was performed according to a published protocol (Houng et al., J Virol Methods 86:1-11 (2000)), except a MyiQ Single-Color Real-Time PCR Detection System (Bio-Rad) with One-Step qRT-PCR Kit (Quanta BioSciences) were used. The DENV2 standard curve was generated with serial dilutions of a known concentration of DENV2 genomic RNA which was in vitro transcribed (MAXlscriptKit, Ambion) from a plasmid containing the cDNA template of S221 3′UTR. After transcription, DNA was digested with DNase I, and RNA was purified using the RNeasy Mini Kit and quantified by spectrophotometry. To control for RNA quality and quantity when measuring DENV in tissues, the level of 18S rRNA was measured using 18S primers described previously (Lacher, et al., Cancer Res 66:1648 (2006)) in parallel real-time RT-PCR reactions. A relative 18S standard curve was made from total splenic RNA.

### Peptide Immunizations

IFN-α/βR^{-/-} mice were immunized s.c. with 50 μg each of NS2B₁₀₈₋₁₂₂, NS3₁₉₈₋₂₁₂, and NS3₂₃₇₋₂₅₁ emulsified in CFA (Difco). After 11 days, mice were boosted with 50 μg peptide emulsified in IFA (Difco). Mock-immunized mice received PBS/DMSO emulsified in CFA or IFA. Mice were infected 13 days after the boost with 10¹¹ GE of DENV2 (some mice were depleted of CD4⁺ or CD8⁺ T cells 3 days and 1 day before infection). Four days later, the mice were sacrificed and tissues harvested, RNA isolated, and DENV2 RNA levels measured as described above. For Example 7, mice were immunized instead with 50 μg each of C₅₁₋₅₉, NS2A₈₋₁₅, NS⁴B₉₉₋₁₀₇, and NS5₂₃₇₋₂₄₅ as described in Yauch et al., J Immunol 182:4865 (2009).

### MHC Peptide-Binding and Restriction Assays

MHC purification and quantitative assays to measure the binding affinity of peptides to purified A*0201, A*0101, A*1101, B*0702 and DRB1*0101 molecules were performed as described elsewhere(Sidney et al., Immunome Res 4:2 (2008); Sidney et al., Curr Protoc Immunol Chapter 18:Unit 18 13 (2001)). Briefly, after a 2-day incubation, binding of the radiolabeled peptide to the corresponding MHC molecule was determined by capturing MHC/peptide complexes on Greiner Lumitrac 600 microplates (Greiner Bio-One, Monroe, N.C.) coated with either the W6/32 (HLA class I specific) or L243 (HLA DR specific) monoclonal antibodies. Bound cpmwere then measured using the Top count microscintillation counter (Packard Instrument, Meriden, Conn.). The concentration of peptide yielding 50% inhibition of the binding of the radiolabeled probe peptide (IC₅₀) was then calculated.

The tumor cell line 721.221(Shimizu et al., J Immunol 142:3320 (1989), which lacks expression of HLA-A, -B and C class I genes, was transfected with the HLA-A*0201/Kb or HL-A*1101 chimeric genes, and was used as APC in the restriction assays. The non-transfected cell line was used as a negative control.

### Human Blood Samples

Peripheral blood samples were obtained from healthy adult blood donors from the National Blood Center in Colombo, Sri Lanka. PBMC were purified by density gradient centrifugation (Ficoll-Hypaque, Amersham Biosciences, Uppsala, Sweden) according to the manufacturer's instructions. Cell were suspended in fetal bovine serum (Gemini Bio-products, Sacramento, Calif.) containing 10% dimethyl sulfoxide, and cryo-preserved in liquid nitrogen. DENV seropositivity was determined by ELISA. A flow cytometry-based neutralization assays was performed for further characterization of seropositve donors, as previously described (Kraus et al., J Clin Microbiol 45:3777 (2007)).

Genomic DNA isolated from PBMC of the study subjects by standard techniques (QIAmp. Qiagen, Valencia, Calif.) was use for HLA typing. High resolution Luminex-based typing for HLA Class I and Class II was utilized according the manufacturer's protocol (Sequence-Specific Oligonucleotides (SSO) typing; One Lambda, Canoga Park, Calif.). Where needed, PCR based methods were used to provide high resolution sub-typing. (Sequence-Specific Primer (SSP) typing; One Lambda, Canoga Park, Calif.).

### IFNγ ELISPOT Assay

For all murine studies, splenic CD4⁺ or CD8⁺ T cells were isolated by magnetic bead positive selection (MiltenyiBiotec, BergischGladbach, Germany) 7 days after infection. 2 ×10⁵ T cells were stimulated with 1×10⁵ uninfected splenocytes as APCs and 10 μg/ml of individual DENV peptides in 96-well flat-bottom plates (Immobilon-P; Millipore, Bedford, Mass.) coated with anti-IFNγ mAb (clone AN18; Mabtech, Stockholm, Sweden). Each peptide was evaluated in triplicate. Following a 20-h incubation at 37° C., the wells were washed with PBS/0.05% Tween 20 and then incubated with biotinylated IFNγ mAb (clone R4-6A2; Mabtech) for 2 h. The spots were developed using Vectastain ABC peroxidase (Vector Laboratories, Burlingame, Calif.) and 3-amino-9-ethylcarbazole (Sigma-Aldrich, St. Louis, Mo.) and counted by computer-assisted image analysis (KS-ELISPOT reader, Zeiss, Munich, Germany). Responses against peptides were considered positive if the net spot-forming cells (SFC) per 10⁶ were ≧20, a stimulation index of ≧2, and p<0.05 in a t test comparing replicates with those from the negative control.

To evaluate the antigenicity of the epitopes in humans, 2×10⁶ PBMC/ml were stimulated in the presence of 1 μg/ml individual peptide for 7 days. Cells were cultured at 37° C., 5% CO₂, and recombinant IL2 (10 U/mL, eBiosciences, San Diego, Calif.) was added 3 days after antigenic stimulation. After one week, PBMC were harvested and tested at a concentration of 1×10⁵/well in an IFNγ ELISPOT assay, as described above. The mAb 1-D1K and mAb 7-B6-1 (Mabtech) were used as coating and biotinylated secondary Ab, respectively. To be considered positive, IFNγ responses needed to exceed the threshold set as the mean responses of HLA non-matched and DENV seronegative donors plus 3 times the standard deviation.

### Statistical Analyses

Data were analyzed with Prism software version 5.0 (GraphPad Software, Inc.). Statistical significance was determined using the unpaired t-test with Welch's correction.

### Example 9

This example includes data demonstrating CD4⁺ T cell activation and expansion following DENV2 infection.

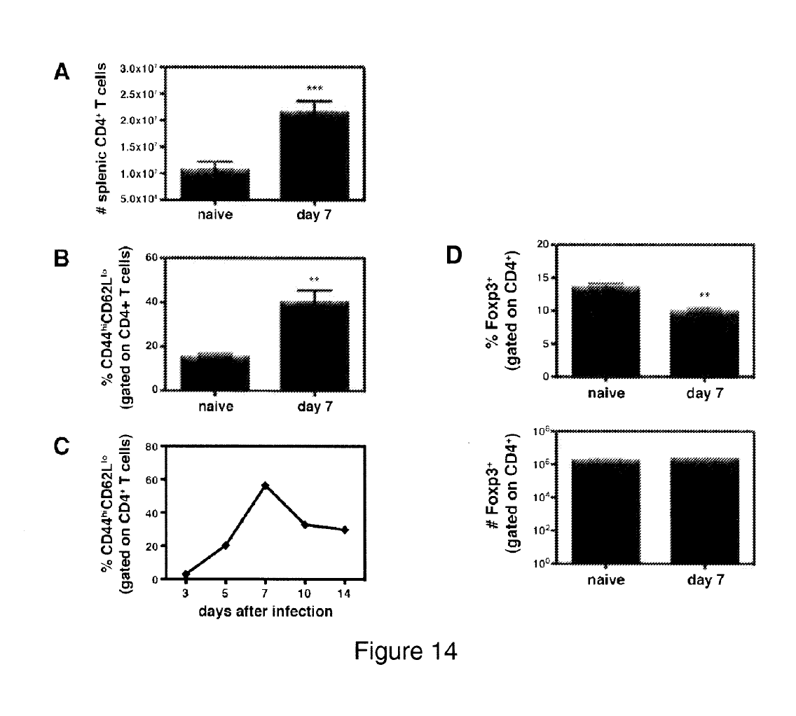

DENV2 (10¹⁰ GE of S221) infection of IFN-α/βR^{-/-} mice results in an acute infection, with viral replication peaking between 2 and 4 days after infection (Yauch, et al. J Immunol 182:4865 (2009)). At this time the mice show signs of disease including hunched posture and ruffled fur, and the virus is subsequently cleared from the serum by day 6. To determine the role of CD4⁺ T cells during the course of this primary DENV2 infection, the expansion of CD4⁺ T cells in the spleens of IFN-α/βR^{-/-} mice 7 days after infection with DENV2 was examined, and a 2-fold increase in CD4⁺ T cell numbers was observed (FIG. 14A). The cells were activated, as measured by CD44 upregulation and CD62L downregulation on splenic CD4⁺ T cells (FIG. 14B) and on circulating blood CD4⁺ T cells, with the peak on day 7 after infection (FIG. 14C). To study the CD4⁺ T cell response in the spleen in more detail, immunohistochemistry on spleen sections obtained from naïve mice and mice 3, 5, and 7 days after DENV2 infection was performed. Sections were stained for CD4, CD8, B220 to highlight B cell follicles, and F4/80 to show red pulp macrophages. As expected, in naïve mice, CD4⁺ and CD8⁺ T cells were dispersed throughout the spleen, but preferentially in T cell areas, also known as the periarteriolar lymphoid sheath (PALS). By day 3 after DENV2 infection, most of the CD4⁺ and CD8⁺ T cells had migrated to the PALS, with very few T cells observed in the red pulp. At day 5, the CD4+ cells were still concentrated in the PALS, at the border between the T cell area and B cell follicles, and also in the B cell follicles. At day 7 after infection, the spleen had increased in size dramatically, and CD4⁺ T cells were found primarily in the PALS and B cell follicles. The localization of CD8⁺ T cells differed from the CD4⁺ T cells mainly in that at day 5 after infection, many of the CD8⁺ T cells had left the T cell area and were found distributed throughout the red pulp and marginal zone (MZ). By day 7, the CD8⁺ T cells were observed in the PALS, MZ, and also the red pulp. These images illustrate the kinetics of the adaptive immune response to DENV2 in the spleen, and show CD4⁺ T cells in close proximity to both CD8⁺ T cells and B cells after DENV2 infection.

Regulatory T cells (Tregs) are a subset of CD4⁺ T cells that are characterized by the expression of the transcription factor, Foxp3 (Josefowicz, et al. Immunity 30:616 (2009)), and have been found to facilitate the early host response to HSV-2 (Lund, et al. Science 320:1220 (2008)) and help control WNV infection (Lanteri, et al. J Clin Invest 119:3266 (2009)). To determine if DENV2 infection resulted in an expansion of Tregs, the number of CD4⁺Foxp3⁺ cells in the spleen 7 days after infection was determined. There was a decrease in the percentage of Tregs among total CD4⁺ cells, and no change in the number of Tregs, demonstrating that DENV2 infection does not lead to an expansion of Tregs in the spleen (FIG. 14D).

### Example 10

This example includes data for the identification of DENV2 CD4+ T cell epitopes and phenotype of DENV2-specific CD4⁺ T cells.

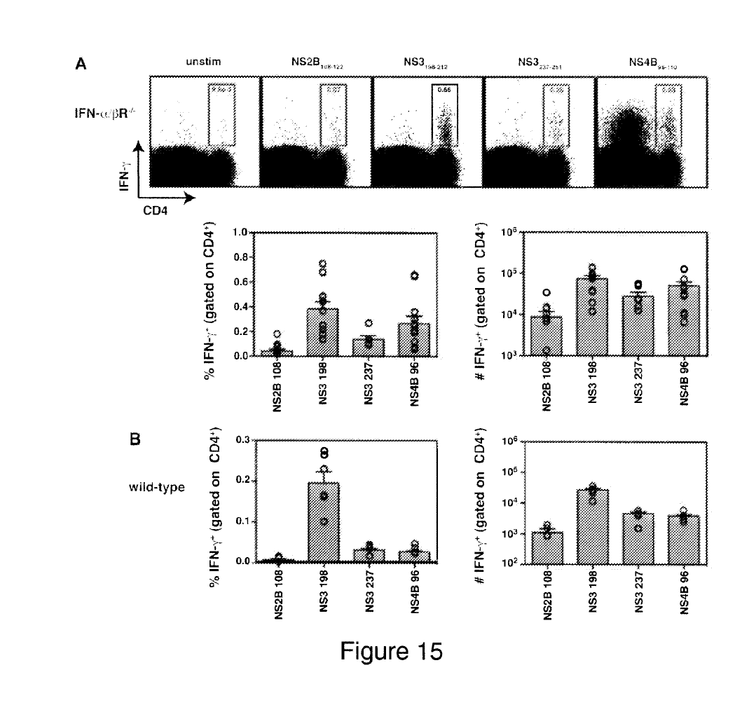

In order to study the DENV2-specific CD4+ T cell response, the identity of MHC class II (I-A^{b})-restricted CD4⁺ T cell epitopes using a bioinformatics prediction method previously reported to map the CD4⁺ T cell response to mouse cytomegalovirus (Arens, et al. J Immunol 180:6472 (2008)) was employed. Briefly, the proteome of DENV2 was screened and 73 15-mer peptides predicted to bind I-A^{b} were identified. The peptides were tested by IFN-γ ICS using splenocytes from DENV2-infected IFN-α/βR^{-/-}mice. Positive peptides (2× background) were then re-ordered as HPLC-purified (>90%) and re-tested. Four positive peptides were identified: NS2B₁₀₈₋₁₂₂, N53₁₉₈₋₂₁₂, N53₂₃₇₋₂₅₁, and NS4B₉₆₋₁₁₀ (FIG. 15A and Table 1). Similar to the DENV2-specific CD8⁺ T cell response (Yauch, et al. J Immunol 182:4865 (2009)), the epitopes identified in IFN-α/βR^{-/-} mice were also recognized by CD4⁺ T cells from DENV2-infected wild-type mice (FIG. 15B), and the magnitude of the CD4⁺ T cell response was higher in IFN-α/βR^{-/-} mice compared with wild-type mice, likely due to increased viral replication. Notably, N53₂₀₀₋₂₁₄ has been identified as a human HLA-DR15-restricted CD4⁺ T cell epitope (Simmons, et al. J Virol 79:5665 (2005); Zeng, et al. J Virol 70:3108 (1996)). It was also of interest that NS4B96-110 contains a CD8⁺ T cell epitope (NS4B₉₉₋₁₀₇) that was identified as the immunodominant epitope in both wild-type and IFN-α/βR^{-/-} C57BL/6 mice infected with DENV2 (Yauch, et al. J Immunol 182:4865 (2009)).

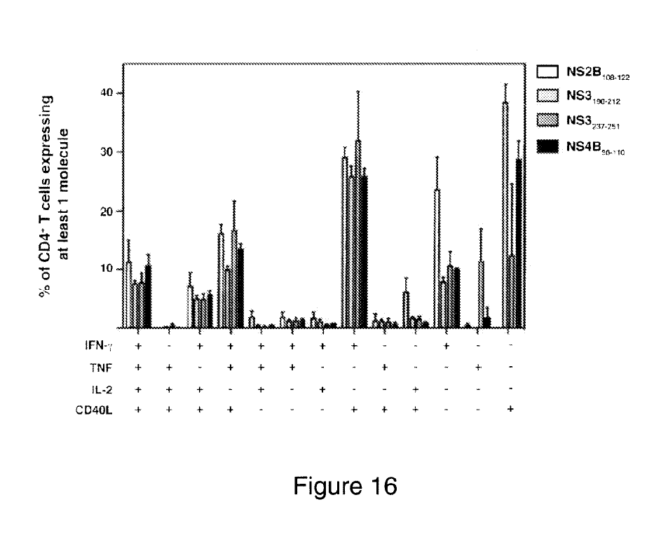

Multicolor flow cytometry was performed to study the phenotype of DENV2-specific CD4⁺ T cells. These cells produced IFN-γ, TNF, and IL-2 (FIG. 16). No intracellular IL-4, IL-5, IL-17, or IL-10 were detected. The DENV2-specific CD4⁺ T cells also expressed CD40L, suggesting they are capable of activating CD40-expressing cells, which include DCs and B cells. The four DENV2-derived CD4⁺ T cell epitopes induced responses that differed in magnitude, but were similar in terms of phenotype. The most polyfunctional cells (those expressing IFN-γ, TNF, IL-2, and CD40L) also expressed the highest levels of the cytokines and CD40L. These results demonstrate that DENV2 infection elicits a virus-specific Th1 CD4⁺ T cell response in IFN-α/βR^{-/-} mice.

TABLE 1
DENV2-derived CD4⁺ T cell epitopes

| Epitope | Sequence |
|---|---|
| NS2B₁₀₈₋₁₂₂ | GLFPVSLPITAAAWY |
| NS3₁₉₈₋₂₁₂ | GKTKRYLPAIVREAI |
| NS3₂₃₇₋₅₁ | GLPIRYQTPAIRAEH |
| NS4B₉₆₋₁₁₀ | IGCYSQVNPITLTAA |

### Example 11

This example includes a description of studies of the effects of CD4⁺ and/or CD8⁺ T cell depletions on DENV2 viral RNA levels, and data showing that CD4⁺ T cells are not required for the anti-DENV2 antibody response, and are also not necessary for the primary DENV2-specific CD8⁺ T cell response.

To determine how CD4⁺ T cells contribute to controlling DENV2 infection, CD4⁺ T cells, CD8⁺ T cells, or both were depleted from IFN-α/βR^{-/-} mice and DENV2 RNA levels 5 days after infection with 10¹⁰ GE of DENV2 was measured. No difference in viral RNA levels between control undepleted mice and CD4-depleted mice in the serum, kidney, small intestine, spleen, or brain was observed (FIG. 17). CD8-depleted mice had significantly higher viral loads than control mice. Depletion of both CD4⁺ and CD8⁺ T cells resulted in viral RNA levels that were significantly higher than those in control mice in all tissues examined, and equivalent to the viral RNA levels in CD8-depleted mice. These data show that CD4+ T cells are not required to control primary DENV2 infection in IFN-α/βR^{-/-} mice, and confirm an important role for CD8⁺ T cells in viral clearance.

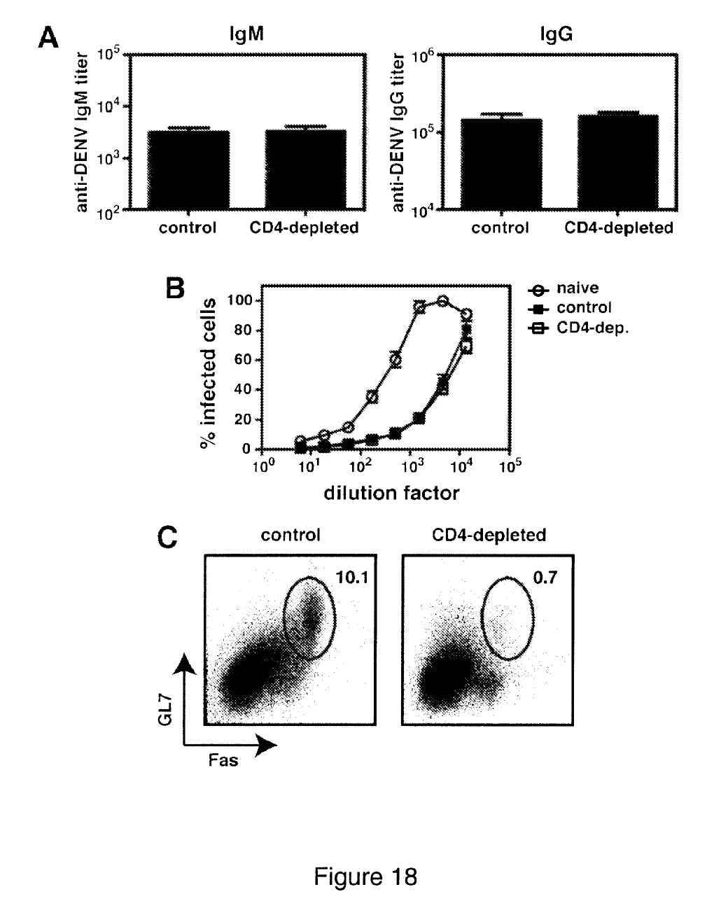

Although CD4⁺ T cells were not required for controlling DENV2 infection, the contribution to the anti-DENV immune response, for example by helping the B cell and/or CD8⁺ T cell responses, was investigated. CSR, the process by which the immunoglobulin heavy chain constant region is switched so the B cell expresses a new isotype of Ab, can be induced when CD40L-expressing CD4⁺ T cells engage CD40 on B cells (Stavnezer, et al. Annu Rev Immunol 26:261 (2008)). However, CSR can also occur in the absence of CD4⁺ T cell help. To determine whether the anti-DENV2 antibody response depends on CD4⁺ T cells, DENV2-specific IgM and IgG titers in the sera of control and CD4-depleted mice was measured 7 days after infection with 10¹⁰ GE of DENV2. As expected, there was no difference in IgM titers at day 7 between control and CD4-depleted mice (FIG. 18A). There was also no difference in IgG titers between control and CD4-depleted mice. To measure the functionality of these DENV2-specific antibody, a flow cytometry-based neutralization assay was performed, in which C6/36 mosquito cells were infected with DENV2 in the presence of heat-inactivated sera obtained from control and CD4-depleted mice 7 days after infection. The sera from control and CD4-depleted mice could neutralize DENV2 equally well (FIG. 18B). As reported previously (Zellweger, et al. Cell Host Microbe 7:128 (2010)), naïve serum was able to prevent DENV infection of C6/36 cells, although not as efficiently as DENV-immune serum. The presence of germinal center (GC) B cells, as the GC reaction is generally thought to be CD4⁺ T cell-dependent (Allen, et al. Immunity 27:190 (2007)), was also evaluated. As expected, GC B cells were absent in the CD4-depleted mice (FIG. 18C). Based on the lack of GC B cells in the DENV2-infected CD4-depleted mice, the early anti-DENV2 antibody response is CD4- and GC-independent.

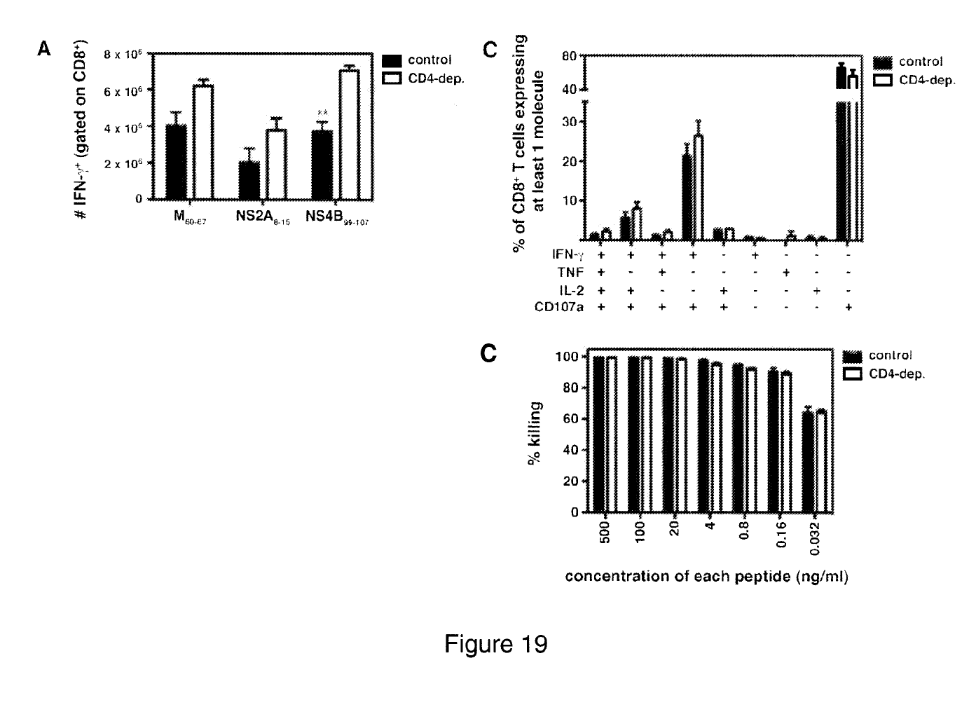

Next, the role of CD4⁺ T cells in helping the CD8⁺ T cell response was assessed by examining the DENV2-specific CD8⁺ T cell response in control and CD4-depleted DENV2-infected mice. The numbers of splenic CD8⁺ T cells were equivalent in control and CD4-depleted mice. IFN-γ ICS was performed using DENV2-derived H-2^{b}-restricted immunodominant peptides identified (M₆₀₋₆₇, NS2A₈₋₁₅, and NS4B₉₉₋₁₀₇) (Yauch, et al. J Immunol 182:4865 (2009)). Somewhat surprisingly, there was an increase in the number of DENV2-specific IFN-γ⁺CD8⁺ T cells in CD4-depleted mice compared with control mice (FIG. 19A). To further characterize the phenotype of the CD8⁺ T cells generated in the absence of CD4⁺ T cells, expression of TNF, IL-2, and CD107a (a marker for degranulation) in cells stimulated with NS4B₉₉₋₁₀₇ was examined (FIG. 19B). As also shown in FIG. 19A, the magnitude of the CD8⁺ T cell response was larger in the CD4-depleted mice, but the cytokine and CD107a expression profiles were comparable. Similar results were observed when cells were stimulated with M₆₀-₆₇ or NS2A₈₋₁₅. Next, the functionality of the DENV2-specific CD8⁺ T cells using an in vivo cytotoxicity assay, in which splenocytes were pulsed with a pool of 4 H-2^{b}-restricted immunodominant peptides and CFSE-labeled before injection into control or CD4-depleted DENV2-infected mice, was examined. CD8⁺ T cell-mediated-cytotoxicity was very efficient; almost 100% killing was observed at peptide concentrations of 500 ng/ml (FIG. 19C). Therefore, the peptide concentrations were titrated down, and no difference in killing was observed between control and CD4-depleted mice at any concentration tested. These data reveal that the primary anti-DENV2 CD8⁺ T cell response, in terms of expansion, cytokine production, degranulation, and cytotoxicity, does not depend on CD4+ T cell help.

### Example 12

This example is a description of studies of in vivo killing of I-A^{b}-restricted peptide-pulsed target cells in DENV2-infected mice, and data showing that vaccination with DENV2 CD4⁺ T cell epitopes controls viral load.

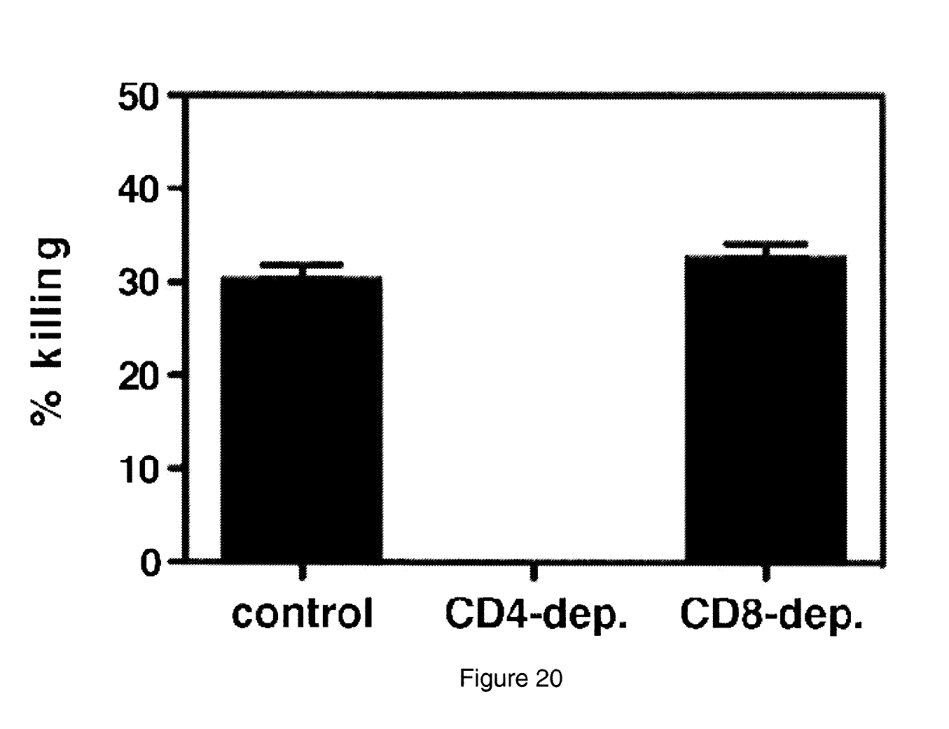

Although the absence of CD4⁺ T cells had no effect on viral RNA levels on day 5 after infection, it was possible that CD4⁺ T cells could still be contributing to the anti-DENV2 host response by killing infected cells. In vivo cytotoxicity assay was performed using splenocytes pulsed with the three peptides that contain only CD4+ T cell epitopes (NS2B₁₀₈₋₁₂₂, NS³₁₉₈₋₂₁₂, and NS3₂₃₇₋₂₅₁) and not NS4B₉₆₋₁₁₀ to measure only CD4⁺, not CD8⁺ T cell-mediated killing. Approximately 30% killing of target cells was observed (FIG. 20). No cytolytic activity was observed when CD4⁺ T cells were depleted, whereas depletion of CD8⁺ T cells had no effect on killing, demonstrating that the cytotoxicity was CD4+ T cell-mediated. Thus, DENV2-specific CD4⁺ T cells exhibit in vivo cytolytic activity, although this effector function does not appear to significantly contribute to controlling primary DENV2 infection.

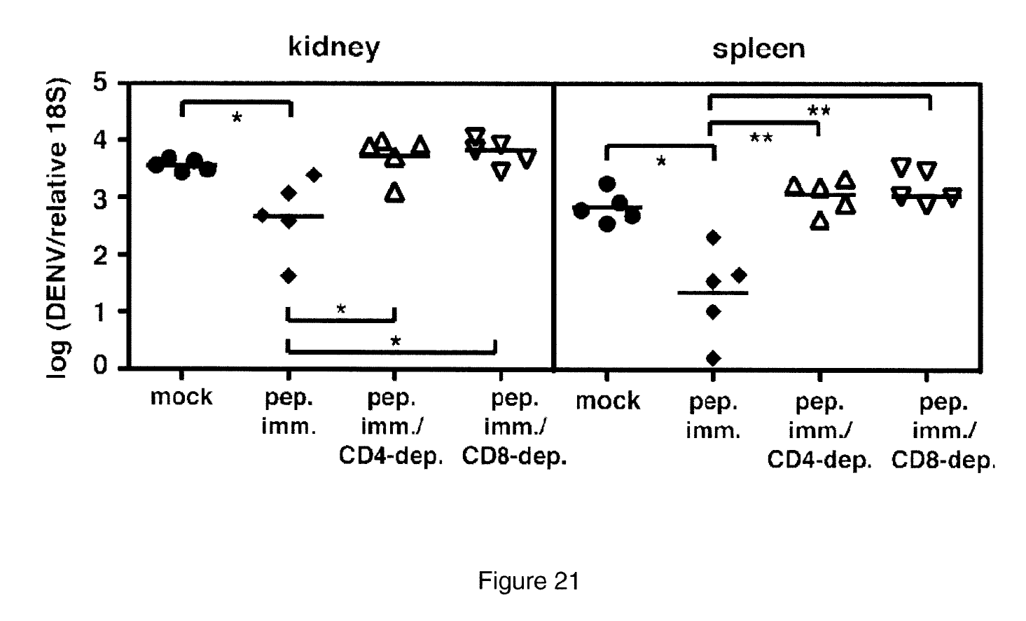

Having found that DENV2-specific CD4⁺T cells can kill target cells, immunization with CD4⁺T cell epitopes was assessed for control of DENV2 infection. Mice were immunized with NS2B₁₀₈₋₁₂₂, NS3₁₉₈₋₂₁₂, and NS3₂₃₇₋₂₅₁ before DENV2 infection, and CD4⁺ T cell responses by ICS and viral RNA levels 4 days after infection measured. Peptide immunization resulted in enhanced CD4⁺ T cell cytokine responses, and significantly lower viral loads in the kidney and spleen (FIG. 21). The protective effect was mediated by CD4⁺ T cells, as CD4-depletion before infection abrogated the protective effect. Similarly, CD8-depletion resulted in no protection, demonstrating that protection after CD4⁺ T cell peptide immunization requires both CD4⁺ and CD8⁺ T cells. These data suggest that CD4+ T cells elicited by immunization protect by helping the CD8⁺ T cell response. Thus, although CD4+ T cells are not required for the primary CD8⁺ T cell or antibody response, and the absence of CD4⁺ T cells had no effect on viral RNA levels, vaccination with CD4⁺ T cell epitopes can reduce viral loads.

### Example 13

This example includes a discussion of the data and a summary of the implications.

The data reveal that CD8⁺ T cells play an important protective role in the response to primary DENV2 infection, whereas CD4⁺ T cells do not. CD4⁺ T cells expanded, were activated, and were located near CD8⁺ T cells and B cells in the spleen after DENV2 infection, yet they did not seem to affect the induction of the DENV2-specific CD8⁺ T cell or antibody responses. In fact, CD4⁺ T cell depletion had no effect on viral clearance. However, the data demonstrate that vaccination with CD4⁺ T cell epitopes prior to DENV infection can provide significant protection, demonstrating that T cell peptide vaccination is a strategy for DENV immunization without the risk of ADE.

The DENV2-specific CD4⁺ T cells recognized epitopes from the NS2B, NS3, and NS4B proteins, and displayed a Th1 phenotype. CD4⁺ T cell epitopes have been identified in mice infected with other flaviviruses, including YFV, for which an I-A^{b}-restricted peptide from the E protein was identified (van der Most, et al. Virology 296:117 (2002)), and WNV, for which six epitopes from the E and NS3 proteins were identified (Brien, et al. J Immunol 181:8568 (2008)). DENV-derived epitopes recognized by human CD4⁺ T cells have been identified, primarily from NS proteins, including the highly conserved NS3 (Mathew, et al. Immunol Rev 225:300 (2008)). One study identified numerous epitopes from the NS3₂₀₀₋₃₂₄ region, and alignment of consensus sequences for DENV1-4 revealed that this region is more conserved (78%) than NS3 as a whole (68%) (Simmons, et al. J Virol 79:5665 (2005)), suggesting that the region contains good candidates for a DENV T cell epitope-based vaccine. Interestingly, one of the NS3-derived epitopes identified herein is also a human CD4⁺ T cell epitope, which may bind human HLAs promiscuously, making it a good vaccine candidate. Another finding was that one of the CD4⁺ T cell epitopes identified in this study contained the most immunodominant of the CD8⁺ T cell epitopes identified previously. Overlapping epitopes have also been found in LCMV (Homann, et al. Virology 363:113 (2007); Mothe, et al. J Immunol 179:1058-1067 (2007); Dow, et al. J Virol 82:11734 (2008)). The significance of overlapping epitopes is unknown, but is likely related to homology between MHC class I and MHC class II, and may be associated with proteasomal processing. Overlapping epitopes may turn out to be common once the complete CD4⁺ and CD8⁺ T cell responses to other pathogens are mapped.

CD4⁺ T cells are classically defined as helper cells, as they help B cell and CD8⁺ T cell responses. However, inflammatory stimuli can override the need for CD4⁺ T cell help, and therefore, the responses to many acute infections are CD4-independent (Bevan, Nat Rev Immunol 4:595 (2004)). DENV2 replicates to high levels in IFN-α/βR^{-/-} mice, the mice appear hunched and ruffled at the time of peak viremia, and they have intestinal inflammation, suggesting that there is a significant inflammatory response to DENV2. Accordingly, CD4⁺ T cells did not play a critical role in the immune response to primary DENV2 infection. The contribution of CD4⁺ T cells has been examined during infections with other flaviviruses. The reports suggest that the contribution of CD4⁺ T cells to protection against flavivirus infection varies depending on the virus and experimental system (Brien, et al. J Immunol 181:8568 (2008); Murali-Krishna, et al. J Gen Virol 77 (Pt 4):705 (1996); Sitati, et al. J Virol. 80:12060 (2006)).

Antibody responses can be T cell-dependent or T cell-independent. In particular, the formation of GCs is thought to be CD4⁺ T cell-dependent, and is where high-affinity plasma cells and memory B cells are generated and where CSR can occur (Stavnezer, et al. Annu Rev Immunol 26:261 (2008); Allen, et al. Immunity 27:190 (2007); Fagarasan et al. Science 290:89 (2000)). T-independent antibody responses to viruses have been demonstrated for vesicular stomatitis virus (Freer, et al. J Virol 68:3650 (1994)), rotavirus (Franco, et al. Virology 238:169 (1997)), and polyomavirus (Szomolanyi-Tsuda, et al. Virology 280:160 (2001)). In addition, EBV (via LMP1) can induce CD40-independent CSR (He, et al. J Immunol 171:5215 (2003)), and mice deficient for CD40 or CD4⁺ T cells are able to mount an influenza-specific IgG response that is protective (Lee, et al. J Immunol 175:5827 (2005)).

The data herein demonstrate that the DENV2-specific IgG response at day 7 is CD4-independent. The lack of GC B cells in CD4-depleted mice shows that CD4-depletions have a functional effect, and indicate anti-DENV IgG is being produced by extrafollicular B cells. It is possible that the absence of CD4⁺ T cells would have an effect on DENV2-specific antibody titers and/or neutralizing activity at later time points, however, the goal of this study was to determine how CD4⁺ T cells contribute to clearance of primary DENV2 infection, and the early anti-DENV2 antibody response is CD4-independent.

Like pathogen-specific antibody responses, primary CD8⁺ T cell responses to many acute infections are also CD4-independent. CD4-independent CD8⁺ T cell responses have been demonstrated for Listeria monocytogenes (Sun, et al. Science 300:339 (2003); Shedlock, et al. J Immunol 170:2053 (2003)), LCMV (Ahmed, et al. J Virol 62:2102 (1988)), and influenza (Belz, et al. J Virol 76:12388 (2002)). Recently a mechanism for how DCs can activate CD8⁺ T cells in the absence of CD4⁺ T cell help has been described (Johnson, et al. Immunity 30:218 (2009)). In accordance with the studies herein, the primary CD8⁺ T cell response to DENV2 did not depend on CD4+ T cells. In fact, an enhanced DENV2-specific CD8⁺ T cell response in CD4-deficient mice compared with control mice at day 7 was observed, which has also been reported for influenza—(Belz, et al. J Virol 76:12388 (2002)) and WNV—(Sitati, et al. J Virol. 80:12060 (2006)) specific CD8⁺ T cell responses. This could be due to the depletion of Tregs, or an increased availability of cytokines (e.g. IL-2) in mice lacking CD4⁺ T cells. This enhanced CD8⁺ T cell response may explain why CD4-depleted mice have no differences in viral titers despite the fact that DENV2-specific CD4⁺ T cells demonstrate in vivo cytotoxicity.

Although CD4⁺ T cells did not play an important role in helping antibody or CD8⁺ T cell responses, DENV2-specific CD4⁺ T cells could kill peptide-pulsed target cells in vivo. CD4⁺ T cells specific for other pathogens, including HIV (Norris, et al. J Virol 78:8844 (2004)) and influenza (Taylor, et al. Immunol Lett 46:67 (1995)) demonstrate in vitro cytotoxicity. In vivo cytotoxicity assays have been used to show CD4⁺ T cell-mediated killing following infection with LCMV (Jellison, et al. J Immunol 174:614 (2005)) and WNV (Brien, et al. J Immunol 181:8568 (2008)). DENV-specific cytolytic human CD4⁺ T cell clones (Gagnon, et al. J Virol 70:141 (1996); Kurane, et al. J Exp Med 170:763 (1989)) and a mouse (H-2^{d}) CD4⁺ T cell clone (Rothman, et al. J Virol 70:6540 (1996)) have been reported. Whether CD4⁺ T cells actually kill infected cells during DENV infection remains to be determined, but is possible, as MHC class II-expressing macrophages are targets of DENV infection (Zellweger, et al. Cell Host Microbe 7:128 (2010)). Based on the fact that CD4-depletion did not have a significant effect on viral clearance, it is unlikely that CD4⁺ T cell-mediated killing plays a major role in the anti-DENV2 response in this model.

A caveat to using the IFN-α/IβR^{-/-} mice is that type I IFNs are known to help T cell and B cell responses through their actions on DCs, and can act directly on T cells (Iwasaki, et al. Nat Immunol 5:987 (2004)). Type I IFNs were found to contribute to the expansion of CD4⁺ T cells following infection with LCMV, but not Listeria monocytogenes (Havenar-Daughton, et al. J Immunol 176:3315 (2006)). Type I IFNs can induce the development of Th1 IFN-γ responses in human CD4⁺ T cells, but cannot substitute for IL-12 in promoting Th1 responses in mouse CD4⁺ T cells (Rogge, et al. J Immunol 161:6567 (1998)). Following Listeria infection, IL-12 synergized with type I IFN to induce IFN-γ production by CD4⁺ T cells (Way, et al. J Immunol 178:4498 (2007)). Although DENV does not replicate to detectable levels in wild-type mice, examining the CD4⁺ T cell response in these mice revealed that the same epitopes were recognized as in the IFN-α/βR^{-/-} mice, but the magnitude of the epitope-specific response was greater in the IFN-α/βR^{-/-} mice. This suggests that the high levels of viral replication in the IFN-α/βR^{-/-} mice are sufficient to drive a DENV2-specific CD4⁺ IFN-γ response. The results demonstrate a DENV2-specific CD4⁺ T cell response, including Th1-type cytokine production and cytotoxicity, in the absence of IFN-α/βR signaling; however, this response is not required for clearance of infection. It is possible that CD4⁺ T cells contribute to protection during DENV infection of hosts with intact IFN responses.

The results herein demonstrate that immunization with CD4⁺ T epitopes is also protective. These results have significant implications for DENV vaccine development, since designing a vaccine is challenging, as, ideally, a vaccine needs to protect against all four serotypes. DENV vaccine candidates in development, some of which are in phase II trials, focus on eliciting an antibody response. The challenge is to induce and maintain a robust neutralizing antibody response against all four serotypes, as it is becoming increasingly clear that non-neutralizing antibodies (or sub-neutralizing quantities of antibodies) can actually worsen dengue disease (Zellweger, et al. Cell Host Microbe 7:128 (2010); Balsitis, et al. PLoS Pathog 6:e1000790 (2010)). An alternative approach would be a peptide vaccine that induces cell-mediated immunity, including both CD4⁺ and CD8⁺ T cell responses, which, although not able to prevent infection, would reduce viral loads and disease severity, and would eliminate the risk of ADE. Such a vaccine should target highly conserved regions of the proteome, for example NS3, NS4B, and/or NS5, and ideally include epitopes conserved across all four serotypes. A vaccine containing only peptides from these particular NS proteins would also preclude the induction of any antibody against epitopes on the virion, which could enhance infection, or antibody against NS1, which could potentially contribute to pathogenesis (Lin, et al. Viral Immunol 19:127 (2006)). Peptide vaccination was given along with CFA, which is commonly used in mice to induce Th1 responses (Billiau, et al. J Leukoc Biol 70:849 (2001)), which was the type of response observed after natural DENV infection. CFA is not a vaccine adjuvant approved for human use, and thus, any peptide vaccine developed against DENV will be formulated with an adjuvant that is approved for human use.

Although the results herein indicate that CD4⁺ T cells do not make a significant contribution to controlling primary DENV2 infection, the characterization of the primary CD4⁺ T cell response and epitope identification allows the determination of the role of CD4⁺ T cells during secondary homologous and heterologous infections. CD4⁺ T cells are often dispensable for the primary CD8⁺ T cell response to infection, but have been shown to be required for the maintenance of memory CD8⁺ T cell responses after acute infection (Sun, et al. Nat Immunol 5:927 (2004)). Finally, the data herein support a DENV vaccine strategy that induces CD4⁺ T cell, in addition to CD8⁺ T cell, responses.

### Example 14

This example includes a description of additional studies showing that vaccination with DENV CD8⁺ T cell epitopes controls viral load.

Since depleting CD8⁺ T cells resulted in increased viral loads and DENV-specific CD8⁺ T cells demonstrated in vivo cytotoxic activity, studies were performed to determine whether enhancing the anti-DENV CD8⁺ T cell response through peptide immunization would contribute to protection against a subsequent DENV challenge. Specifically, the effect of peptide vaccination on viremia was determined by immunizing IFN-α/βR^{-/-} mice with DENV-2 derived H-2^{b} peptides prior to infection with S221. Mice were immunized with four dominant DENV epitopes (C₅₁₋₅₉, NS2A₈₋₁₅, NS4B₉₉₋₁₀₇, and N55₂₃₇₋₂₄₅) (Yauch et al., J Immunol 182:4865 (2009)) in an attempt to induce a multispecific T cell response, which is desirable to prevent possible viral escape through mutation (Welsh et al., Nat Rev Microbiol 5:555 (2007)). At day 4 after infection, viremia in the serum was measured by real-time RT-PCR, as described above. The peptide-immunization resulted in enhanced control of DENV infection, with 350-fold lower serum DENV RNA levels in peptide-immunized mice than mock-immunized mice (Yauch et al., J Immunol 182:4865 (2009)). To confirm that the protection was mediated by CD8⁺ T cells, CD8⁺ T cells were depleted from a group of peptide-immunized mice prior to infection, and it was found that this abrogated the protective effect (Yauch et al., J Immunol 182:4865 (2009)). Thus, the data demonstrate that a preexisting DENV-specific CD8⁺ T cell response induced by peptide vaccination enhances viral clearance.

Most dengue infections are asymptomatic or classified as DF, whereas DHF/DSS accounts for a small percentage of dengue cases, indicating that in most infections the host immune response is protective. These data indicate that CD8⁺ T cells contribute to protection during primary infection by reducing viral load and that CD8⁺ T cells are an important component to a protective immune response.

This study shows that immunization with four dominant epitopes prior to infection resulted in enhanced DENV clearance, and this protection was mediated by CD8⁺ T cells. These results indicate that vaccination with T cell epitopes can reduce viremia.

Results from the Examples described herein reveal a critical role for CD8⁺ T cells in the immune response to an important human pathogen, and provide a rationale for the inclusion of CD8⁺ T cell epitopes in DENV vaccines. Furthermore, identification of the CD8⁺ T cell epitopes recognized during DENV infection in combination with the disclosed mouse model can provide the foundation for elucidating the protective versus pathogenic role of CD8⁺ T cells during secondary infections.

### Example 15

This example is a description of a novel system to identify DENV specific HLA*0201 epitopes.

Mouse-passaged DENV is able to replicate to significant levels in IFN-α/βR^{-/-} mice. HLA*0201 transgenic and IFN-α/βR^{-/-} mice strains were backcrossed to study DENV-specific HLA restricted T cell responses. These mice were then infected with mouse adapted DENV2 strain S221, and purified splenic T cells were used to study the anti-DENV CD8⁺ T cell responses.

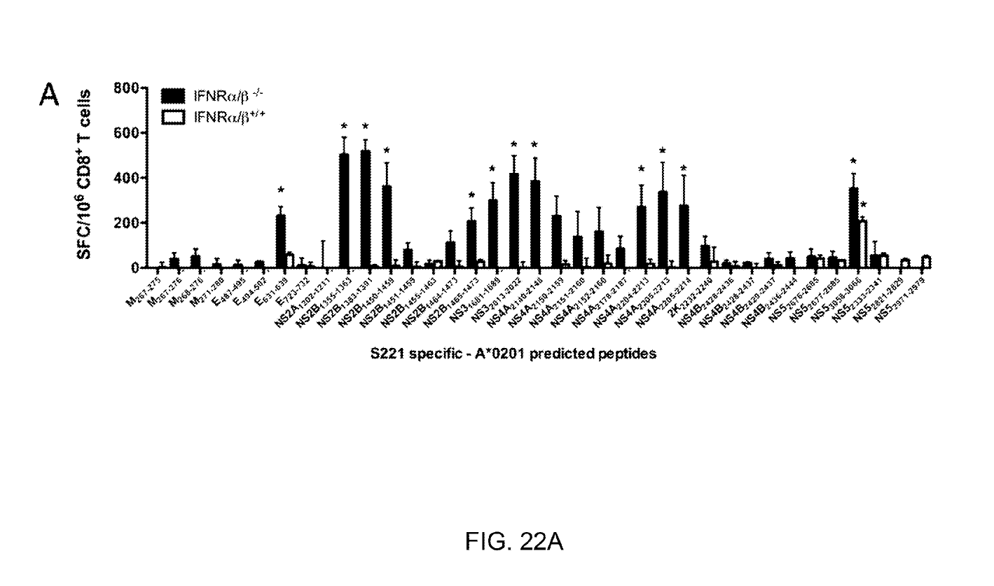

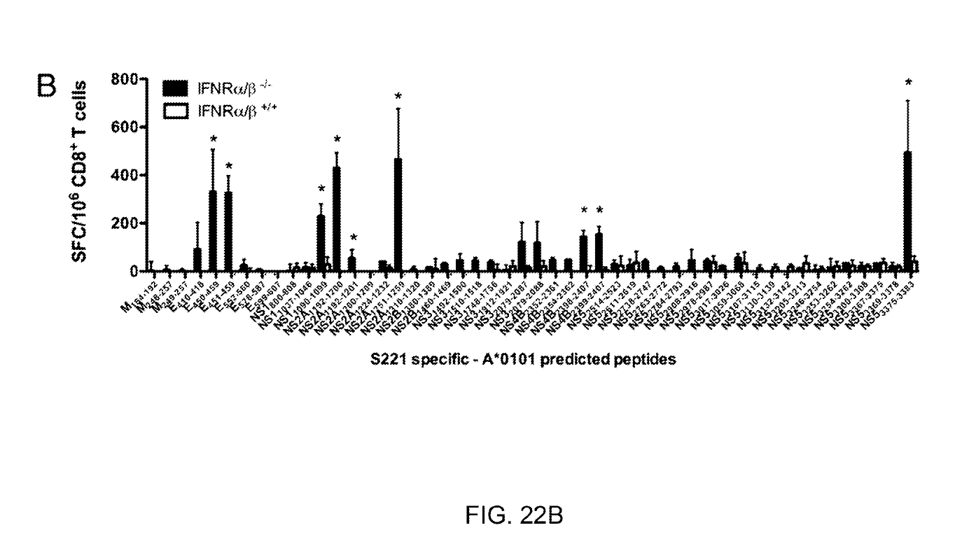

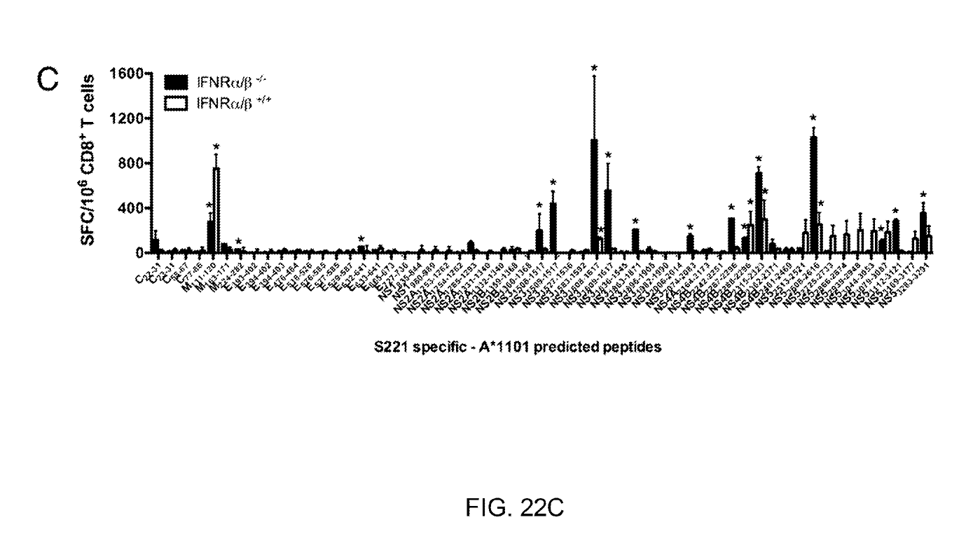

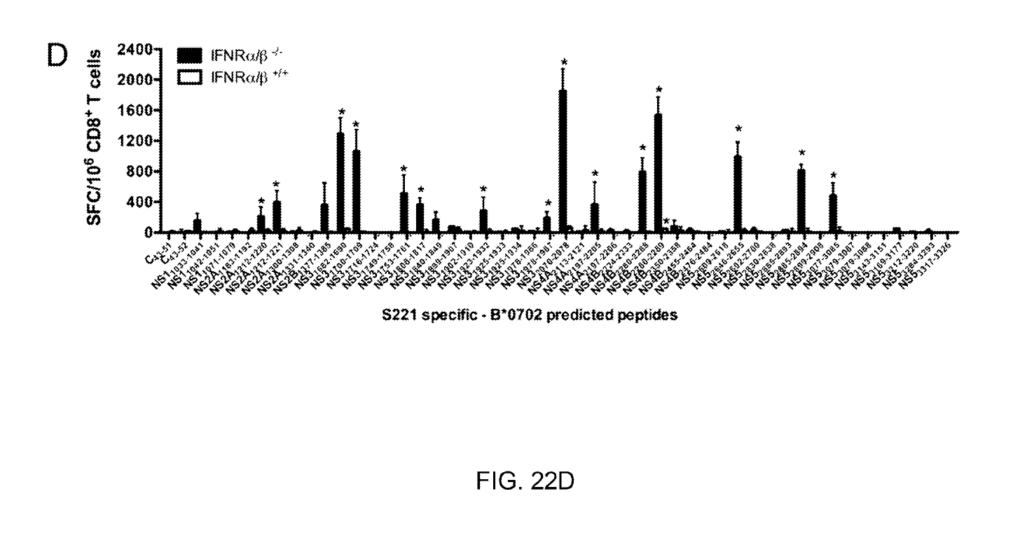

A panel of 116 predicted A*0201 binding peptides were generated using bioinformatics (Moutaftsi, et al. Nat Biotechnol 24:817 (2006)). Predicted HLA A*0201 binding peptides were combined into pools of 10 individual peptides and tested in an IFNγ ELISPOT assay using CD8⁺ T cells from HLA transgenic IFN-α/⊕R^{-/-} and IFN-α/βR^{+/+}, S221 infected mice, respectively. Positive pools were deconvoluted and the individual peptides were tested in two independent studies. Using this approach, a single peptide in the HLA*A0201 IFN-α/βR^{+/+} mice was identified (NS5₃₀₅₈₋₃₀₆₆, FIG. 22A, white bars) whereas screening in IFN-α/βR^{-/-} mice lead to identification of ten additional epitopes. (FIG. 22A, black bars.) These results demonstrate that the HLA A transgenic IFN-α/βR^{-/-} has a stronger and broader T cell response.

### Example 16

This example describes population coverage by additional HLA transgenic mice IFN-α/βR−/− strains.

To address whether similar observations could be made by assessing responses in other HLA-transgenic IFN-α/βR^{-/-} and IFN-α/βR^{-/-} mice, IFN-α/βR^{-/-} mice were backcrossed with HLA A*0101, A*1101, and B*0702 transgenic mice. These alleles were chosen as representatives of three additional HLA class I supertypes (A1, A3 and B7, respectively).

Screening in HLA A*0101 and A*1101 transgenic IFN-α/βR^{-/-} mice revealed 9 HLA A*0101 restricted (FIG. 22B, black bars), and 16 A*1101 restricted epitopes (FIG. 22C, black bars), respectively. In case of the HLA A*0101 transgenic wildtype mice, no epitope could be detected, whereas the HLA A*1101 transgenic mice showed an overlap of 5 epitopes with the corresponding IFN-α/βR^{-/-} strain (M₁₁₁₋₁₂₀, NS3₁₆₀₈₋₁₆₁₇, NS4B₂₂₈₇₋₂₂₉₆, NS⁴B₂₃₁₅₋₂₃₂₃ and N55₃₁₁₂₋₃₁₂₁). Two of these epitopes were able to elicit a stronger response in the HLA A*1101 IFN-α/βR^{+/+} mice compared to the IFN-α/βR^{-/-} strain (M₁₁₁₋₁₂₀ and NS4B₂₂₈₇₋₂₂₉₆). All other responses observed were stronger in the IFN-α/βR^{-/-} mice.

To extend the observations to mice transgenic for an HLA B allele HLA B*0702 transgenic IFN-α/βR^{-/-} and IFN-α/βR^{+/+} mice were infected and epitope recognition was compared between the two strains. 15 B*0702 restricted epitopes in the IFN-α/βR^{-/-} strain (FIG. 22D, black bars) were identified. 1 of these has also been detected in the corresponding IFN-α/βR^{+/+} mice (NS4B₂₂₈₀₋₂₂₈₉; FIG. 22D, white bars). Similar to the other HLA transgenic mouse strains, the responses observed in the HLA B*0702 transgenic IFN-α/βR^{-/-} mice were not only broader but also more than ten-fold higher in magnitude. The one epitope recognized in the IFN-α/βR^{+/+} strain elicited an IFNγy response of 50 SFC/10⁶ CD8⁺ T cells compared to an average of 857 SFC/10⁶ CD8⁺ T cells in the IFN-α/βR^{-/-} mice.

### Example 17

This example describes Dengue virus specific T cell responses in an MHC class II transgenic mouse model.

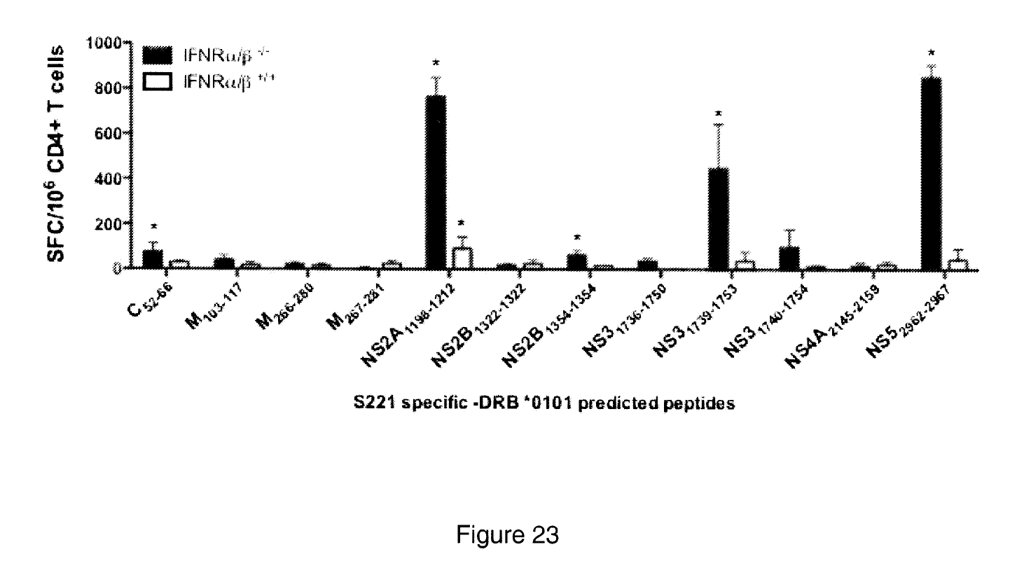

To determine if the observations made in the case of MHC class I transgenic mice were also applicable to MHC class II molecules, the antigenicity of HLA DRB1*0101 DENV predicted binding peptides in HLA DRB1*0101, IFN-α/βR^{-/-} and IFN-α/βR^{+/+} mice, respectively, was determined. Using the same study conditions described above for the MHC class I transgenic mice, HLA DRB1*0101, IFN-α/βR^{-/-} and IFN-α/βR^{+/+} mice were infected with DENV2 (S221), and CD4⁺ T cells were isolated 7 days post infection. A panel of 12 predicted S221 specific peptides was then analyzed for IFNγ production by ELISPOT. Five epitopes in the DRB1*0101, IFN-α/βR^{-/-} mice were identified from these assays which could elicit a significant IFNγ response in two independent studies (FIG. 23; black bars). As seen above in the MHC class I transgenic mice, only one peptide could be detected in the corresponding DRB1*0101, IFN-α/βR^{+/+} mice (NS2A₁₁₉₉₋₁₂₁₃; FIG. 23, white bars). This identified epitope in the IFN-α/βR^{+/+} did not represent a novel epitope as it was also observed in the corresponding IFN-α/βR^{-/-} mice. Similarly to the MHC class I transgenic mice all observed responses were stronger in the IFN-α/βR^{-/-} mice.

In summary, a total of 55 epitopes were identified in the HLA transgenic IFN-α/βR^{-/-} mice, whereas the same screen in HLA transgenic IFN-α/βR^{+/+} mice only revealed 8 epitopes. All of these 8 epitopes have also been detected in the HLA transgenic IFN-α/βR^{-/-} mice. The broader repertoire seen in IFN-α/βR^{-/-} mice as well as the stronger and more robust IFNγ responses, suggest that HLA transgenic mice, backcrossed with IFN-α/βR^{-/-} mice are a more suitable model to study T cell responses to DENV infection than HLA transgenic wildtype mice.

### Example 18

This example is a description of mapping optimal epitopes with respect to peptide length, and further characterization of the identified epitopes.

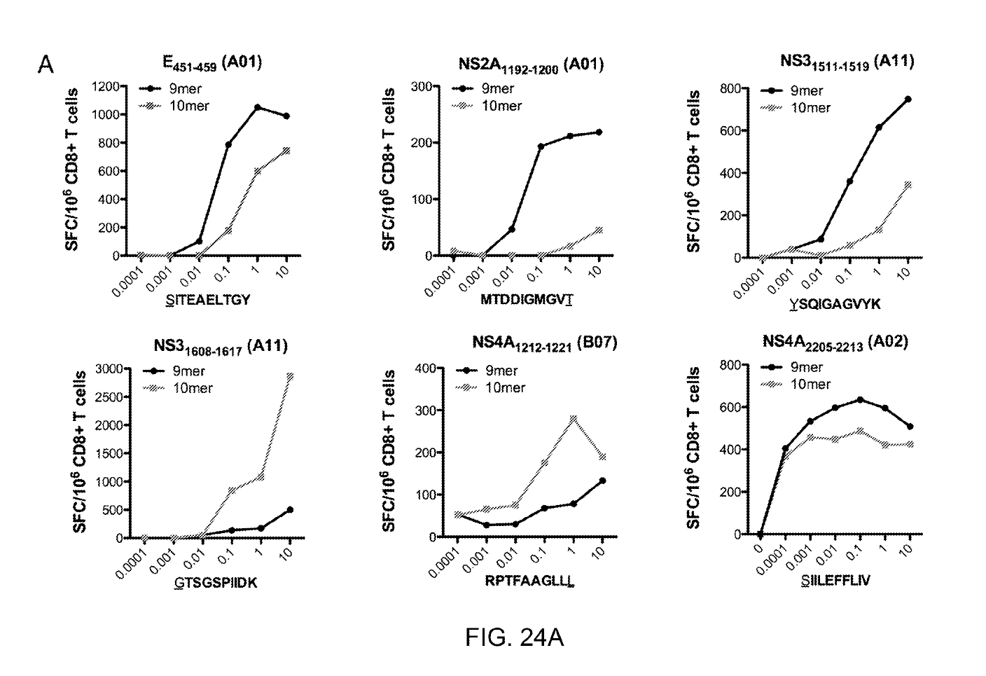

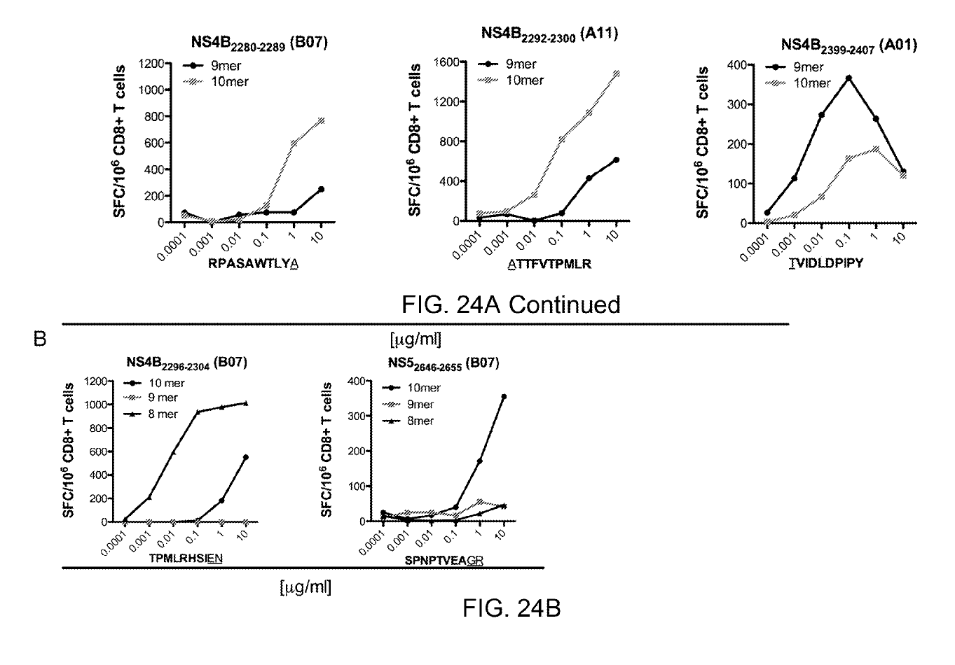

For all HLA alleles tested in this study, class I 9- and 10-mer peptide predictions were performed using the consensus prediction tool as described in greater detail in Example 1. Within the 50 MHC class I restricted epitopes identified, 9 pairs of nested epitopes were identified, where the 9-mer as well as the 10-mer peptide was able to elicit an immune response. To determine which specific peptide within each nested epitope pair was the optimal epitope, peptide titration assays were employed (FIG. 24A). For one epitope (NS4A₂₂₀₅₋₂₂₁₃), both the 9- and the 10-mer displayed similar kinetics upon peptide titration (FIG. 24A). Since the 9-mer was able to elicit slightly higher responses in all conditions tested, the 9-mer version of this epitope was used for further studies. In all other cases an optimal epitope length peptide could be unequivocally identified.

Similarly, for two of the identified B*0702 restricted epitopes (NS4B₂₂₉₆₋₂₃₀₅ and NS5₂₆₄₆₋₂₆₅₅) which showed low binding affinity (IC₅₀>1000 nM) 8-, and 9-mers carrying alternative dominant B7 motifs were synthesized and tested them for T cell recognition and binding affinity. In one case the corresponding 8-mer (NS4B₂₂₉₆₋₂₃₀₄) showed dominant IFNγ responses as well as higher binding affinity compared to the 9-mer. In the other case, the l0 mer originally identified (NS5₂₆₄₆₋₂₆₅₅) was able to elicit higher responses than the newly synthesized 8- and 9-mer. In both cases the optimal epitope length could be identified and was considered further in the study, as shown in FIG. 24B.

Of all five HLA transgenic mouse strains analyzed, two strains, namely the A*0201 and the A*1101 transgenic strains, co-expressed murine MHC molecules together with the respective HLA molecule. Thus it was necessary to address that the observed responses were restricted by the human HLA class I molecule and not by murine Class I. Accordingly, purified T cells were studied for their capacity to recognize the specific epitopes when pulsed on antigen presenting cells expressing only human HLA class and not any murine class I molecule. For this purpose, the tumor cell line 721.221 was utilized, which is negative for expression of any human or murine Class I molecule, and was transfected with either HLA A*0201 or HLA*1101.

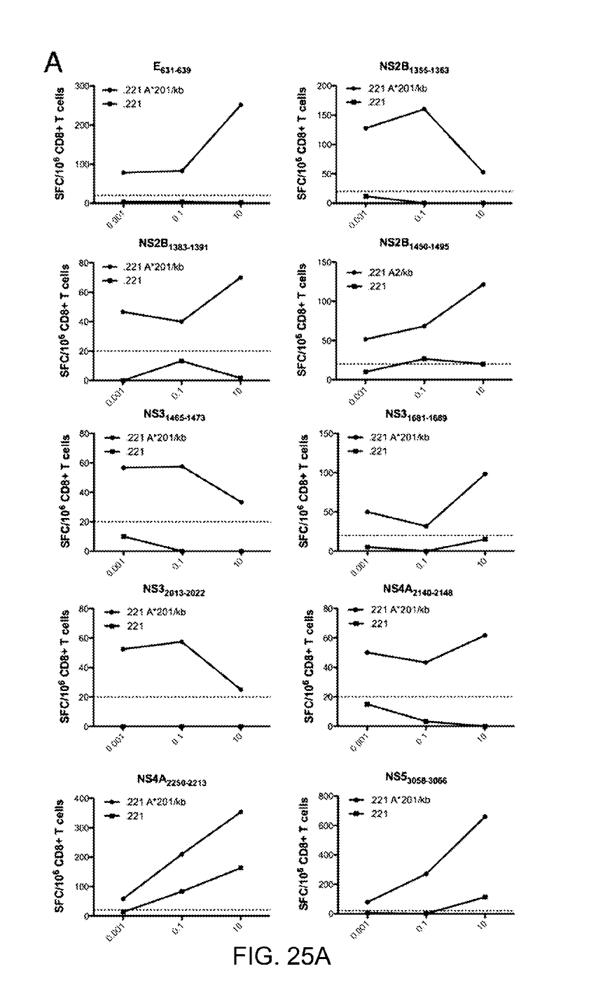

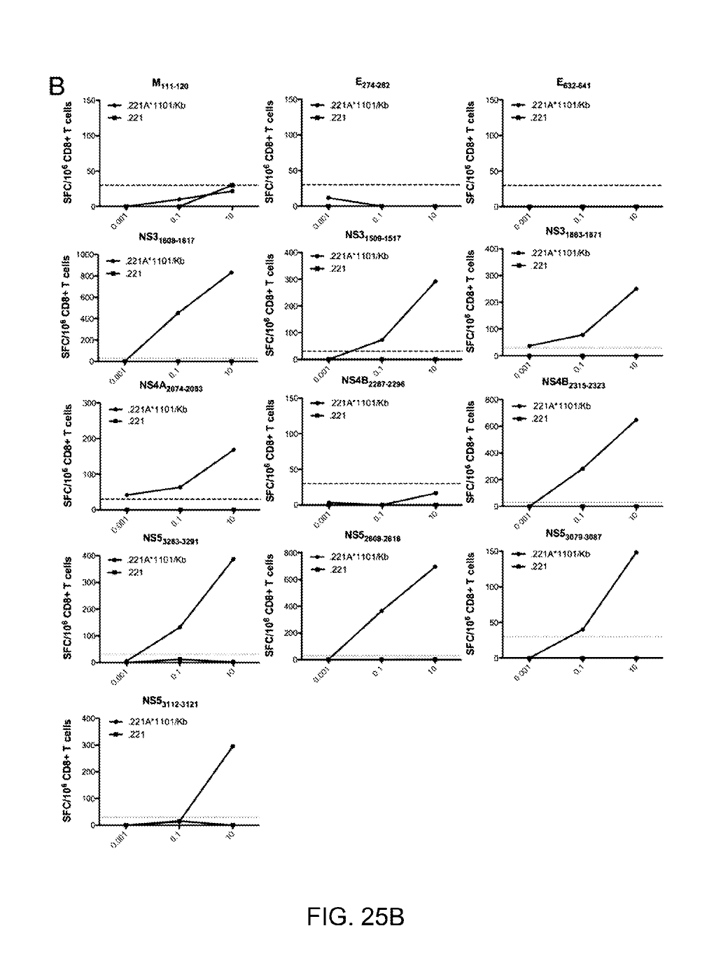

As shown in FIG. 25A, all ten HLA*A0201 restricted epitopes were recognized when presented by APC exclusively expressing HLA*A0201 molecules. Nine out of thirteen of the HLA*A1101 restricted epitopes identified did stimulate a CD8⁺ T cell response when presented exclusively on HLA*1101 molecules (FIG. 25B). When the four remaining epitopes were tested in non-HLA transgenic IFN-α/βR^{-/-} mice as described above, all elicited a significant T cell response. Furthermore, one of the epitopes has already been described to be recognized by T cells from DENV2 infected Balb/c mice (E₆₃₃₋₆₄₂ (Roehrig, et al. J Virol 66:3385 (1992))). These epitopes (M₁₁₁₋₁₂₀, E₂₇₄₋₂₈₂, E₆₃₃₋₆₄₂, NS4B₂₂₈₇₋₂₂₉₆) are therefore considered solely mouse MHC restricted, and were excluded from further study. Among those epitopes were also the two epitopes which elicited a stronger response in the HLA A*1101 IFN-α/βR^{+/+} mice compared to the IFN-α/βR^{-/-} strain (M₁₁₁₋₁₂₀ and NS4B₂₂₈₇₋₂₂₉₆).

To further confirm the MHC restriction of the identified epitopes, MHC-binding capacity to their predicted allelic molecule was measured using purified HLA molecules in an in vitro binding assay. The results of these assays are also shown in Table 2. 32 of the 42 tested peptides (67%) bound the corresponding predicted allele with high affinity as indicated by an IC₅₀<50 nM. 16 out of these even showed an IC₅₀<10 nM and can therefore be considered as very strong binders. Of the remaining peptides, 7 (17%) were able to bind the predicted allele with intermediate affinities as indicated by IC₅₀<150 nM. Only three of the identified epitopes (7%) bound with low affinity, showing an IC₅₀>500nM. A summary of all epitopes identified, after conclusion of the studies and elimination of redundancies, is shown in Table 2.

TABLE 2
Identified DENV2 derived epitopes in HLA-transgenic IFN-α/βR^{-/-} mice

| | | | | | | | Coservancy | | | | |
|---|---|---|---|---|---|---|---|---|---|---|---|
| | | | T cell | | | | within | | | | |
| | | | repsonses | | | HLA | stereotypes [%] | | | | |
| | | Restric- | [SFC] | | frequency | binding | DEN | DEN | DEN | DEN | |
| Epitope | Sequence | tion | mouse | human | in humans | [IC₅₀] | V2 | V1 | V3 | V4 | References |
| E₄₅₁₋₄₅₉ | ITEAELTGY | A*0101 | 327 | 67 | 20% (1 out | 25 | 85 | 0 | 0 | 0 | |
| | | | | | of 5) | | | | | | |
| NS1₁₀₉₀₋₁₀₉₉ | RSCTLPPLRY | | 228 | 104 | 20% (1 out | 5.9 | 100 | 0 | 100 | 0 | |
| | | | | | of 5) | | | | | | |
| NS2A₁₁₉₂₋₁₂₀₀ | MTDDIGMGV | | 430 | 163 | 20% (1 out | 19 | 84 | 0 | 0 | 0 | |
| | | | | | of 5) | | | | | | |
| NS2A₁₂₅₁₋₁₂₅₉ | LTDALALGM | | 465 | 143 | 40% (2 out | 129 | 91 | 0 | 0 | 0 | |
| | | | | | of 5) | | | | | | |
| NS4B₂₃₉₉₋₂₄₀₇ | VIDLDPIPY | | 153 | 92 | 20% (1 out | 17 | 53 | 0 | 0 | 0 | |
| | | | | | of 5) | | | | | | |
| NS5₃₃₇₅₋₃₃₈₃ | YTDYMPSMK | | 495 | 143 | 20% (1 out | 37 | 98 | 0 | 0 | 0 | |
| | | | | | of 5) | | | | | | |
| E₆₃₁₋₆₃₉ | RLITVNPIV | A*0201 | 265 | 393 | 43% (3 out | 2.8 | 98 | 0 | 0 | 0 | |
| | | | | | of 7) | | | | | | |
| NS2B₁₃₅₅₋₁₃₆₃ | IMAVGMVSI | | 503 | 417 | 43% (3 out | 1.9 | 92 | 0 | 0 | 0 | |
| | | | | | of 7) | | | | | | |
| NS2B₁₃₈₃₋₁₃₉₁ | GLLTVCYVL | | 519 | 434 | 57% (4 out | 6.0 | 100 | 0 | 0 | 0 | |
| | | | | | of 7) | | | | | | |
| NS2B₁₄₅₀₋₁₄₅₉ | LLVISGLFPV | | 361 | 588 | 43% (3 out | 26 | 50 | 0 | 0 | 0 | |
| | | | | | of 7) | | | | | | |
| NS3₁₄₆₅₋₁₄₇₃ | AAAWYLWEV | | 207 | 495 | 57% (4 out | 0.39 | 92 | 0 | 0 | 0 | |
| | | | | | of 7) | | | | | | |
| NS3₁₆₈₁₋₁₆₈₉ | YLPAIVREA | | 299 | 401 | 71% (5 out | 18 | 99 | 0 | 0 | 0 | [76¹] |
| | | | | | of 7) | | | | | | |
| NS3₂₀₁₃₋₂₀₂₂ | DLMRRGDLPV | | 417 | 312 | 71% (5 out | 6.3 | 92 | 0 | 0 | 0 | |
| | | | | | of 7) | | | | | | |
| NS4A₂₁₄₀₋₂₁₄₈ | ALSELPETL | | 384 | 297 | 14% (1 out | 61 | 99 | 0 | 0 | 0 | [77²] |
| | | | | | of 7) | | | | | | |
| NS4A₂₂₀₅₋₂₂₁₃ | IILEFFLIV | | 336 | 301 | 28% (2 out | 18 | 99 | 0 | 0 | 0 | |
| | | | | | of 7) | | | | | | |
| NS5₃₀₅₈₋₃₀₆₆ | KLAEAIFKL | | 353 | 597 | 43% (3 out | 2.2 | 95 | 0 | 0 | 0 | [77] |
| | | | | | of 7) | | | | | | |
| NS3₁₅₀₉₋₁₅₁₇ | SQIGAGVYK | A*1101 | 436 | 0 | 0% (0 out of | 33 | 98 | 0 | 0 | 0 | |
| | | | | | 5) | | | | | | |
| NS3₁₆₀₈₋₁₆₁₇ | GTSGSPIIDK | | 1003 | 880 | 20% (1 out | 12 | 30 | 0 | 0 | 0 | [78³] |
| | | | | | of 5) | | | | | | |
| NS3₁₈₆₃₋₁₈₇₁ | KTFDSEYVK | | 208 | 0 | 0% (0 out of | 140 | 75 | 0 | 0 | 0 | [76] |
| | | | | | 5) | | | | | | |
| NS4A₂₀₇₄₋₂₀₈₃ | RIYSDPLALK | | 148 | 3087 | 20% (1 out | 51 | 89 | 0 | 0 | 0 | [76] |
| | | | | | of 5) | | | | | | |
| NS4B₂₃₁₅₋₂₃₂₃ | ATVLMGLGK | | 712 | 0 | 0% (0 out of | 16 | 98 | 0 | 0 | 0 | |
| | | | | | 5) | | | | | | |
| NS5₂₆₀₈₋₂₆₁₆ | STYGWNLVR | | 1030 | 0 | 0% (0 out of | 22 | 100 | 0 | 0 | 0 | |
| | | | | | 5) | | | | | | |
| NS5₃₀₇₉₋₃₀₈₇ | TVMDIISRR | | 105 | 0 | 0% (0 out of | 71 | 91 | 0 | 0 | 0 | |
| | | | | | 5) | | | | | | |
| NS5₃₁₁₂₋₃₁₂₁ | RQMEGEGVFK | | 284 | 0 | 0% (0 out of | 118 | 43 | 0 | 0 | 0 | |
| | | | | | 5) | | | | | | |
| NS5₃₂₈₃₋₃₂₉₁ | RTTWSIHAK | | 358 | 800 | 20% (1 out | 83 | 65 | 0 | 0 | 0 | |
| | | | | | of 5) | | | | | | |
| NS2A₁₂₁₂₋₁₂₂₁ | RPTFAAGLLL | B*0702 | 400 | 335 | 20% (1 out | 4.8 | 92 | 0 | 0 | 0 | |
| | | | | | of 5) | | | | | | |
| NS3₁₆₈₂₋₁₆₉₀ | LPAIVREAI | | 1293 | 207 | 20% (1 out | 6.5 | 100 | 98 | 96 | 0 | [76] |
| | | | | | of 5) | | | | | | |
| NS3₁₇₀₀₋₁₇₀₉ | APTRVVAAEM | | 1064 | 1426 | 40% (2 out | 4.6 | 99 | 0 | 100 | 100 | [76] |
| | | | | | of 5) | | | | | | |
| NS3₁₇₅₃₋₁₇₆₁ | VPNYNLIIM | | 509 | 410 | 20% (1 out | 43 | 100 | 0 | 89 | 0 | |
| | | | | | of 5) | | | | | | |
| NS3₁₈₀₈₋₁₈₁₇ | APIMDEEREI | | 364 | 232 | 20% (1 out | 572 | 77 | 0 | 0 | 0 | |
| | | | | | of 5) | | | | | | |
| NS3₁₉₇₈₋₁₉₈₇ | TPEGIIPSMF | | 194 | 1825 | 20% (1 out | 589 | 99 | 0 | 0 | 0 | [76] |
| | | | | | of 5) | | | | | | |
| NS3₂₀₇₀₋₂₀₇₈ | KPRWLDARI | | 1853 | 1633 | 40% (2 out | 6.8 | 91 | 0 | 0 | 0 | [76] |
| | | | | | of 5) | | | | | | |
| NS4B₂₂₈₀₋₂₂₈₉ | RPASAWTLYA | | 1539 | 0 | 0% (0 out of | 7.4 | 100 | 37 | 0 | 100 | |
| | | | | | 5) | | | | | | |
| NS4B₂₂₉₆₋₂₃₀₄ | TPMLRHSI | | 1013 | 460 | 20% (1 out | 1.1 | 100 | 0 | 0 | 0 | |
| | | | | | of 5) | | | | | | |
| NS5₂₆₄₆₋₂₆₅₅ | SPNPTVEAGR | | 994 | 0 | 0% (0 out of | 1332 | 54 | 0 | 0 | 0 | |
| | | | | | 5) | | | | | | |
| NS5₂₈₈₅₋₂₈₉₄ | TPRMCTREEF | | 811 | 1341 | 60% (3 out | 13 | 89 | 0 | 0 | 0 | |
| | | | | | of 5) | | | | | | |
| NS5₃₀₇₇₋₃₀₈₅ | RPTPRGTVM | | 487 | 390 | 40% (2 out | 1.5 | 97 | 0 | 0 | 0 | |
| | | | | | of 5) | | | | | | |
| C₅₃₋₆₇ | AFLRFLTIPPTAG | DRB1*01 | 77 | 314 | 75% (3 out | 9.7 | 99 | 0 | 0 | 0 | [79⁴] |
| | IL | 01 | | | of 4) | | | | | | |
| NS2A₁₁₉₉₋₁₂₁₃ | GVTYLALLAAFKV | | 764 | 249 | 75% (3 out | 10 | 91 | 0 | 0 | 0 | |
| | RP | | | | of 4) | | | | | | |
| NS2B₁₃₅₆₋₁₃₇₀ | MAVGMVSILASSL | | 65 | 279 | 75% (3 out | 34 | 100 | 0 | 0 | 0 | |
| | LK | | | | of 4) | | | | | | |
| NS3₁₇₄₂₋₁₇₅₆ | TFTMRLLSPVRVP | | 448 | 336 | 75% (3 out | 1.5 | 70 | 100 | 99 | 0 | [76] |
| | NY | | | | of 4) | | | | | | |
| NS5₂₉₆₆₋₂₉₈₀ | SRAIWYMWLGAR | | 851 | 729 | 75% (3 out | 17 | 100 | 99 | 0 | 100 | |
| | FLE | | | | of 4) | | | | | | |

¹[76] Simmons et al., J Virol 79:5665 (2005)
²[77] Appanna et al,. Clin Vaccine Immunol 14:969 (2007)
³[78] Mongkolsapaya et al., J Immunol 176:3821
⁴[79] Wen et al., Virus Res 132:42 (2008)

### Example 19

This example includes a description of validation studies of the identified epitopes in human DENV seropositive donors.

To validate the epitopes identified in the HLA-transgenic IFN-α/βR^{-/-} mice, the capacity of these epitopes to stimulate PBMC from human donors, previously exposed to DENV, was analyzed. Since the IFNγ response to these peptides was not detectable ex vivo, HLA-matched PBMC were re-stimulated for 7 days in presence of the respective peptides and IL2. As a control PBMC from donors which neither expressed the exact HLA-molecule nor one from the same supertype, as well as PBMC from DENV seronegative donors were re-stimulated. The average IFNγ response from these donors plus 3 times the standard deviation (SD) was set as a threshold of positivity.

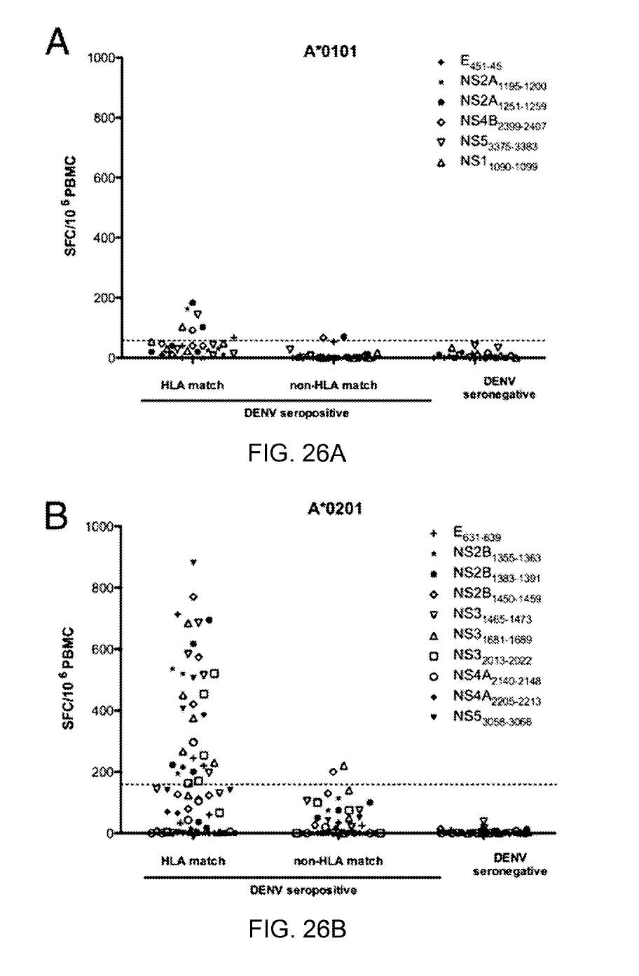

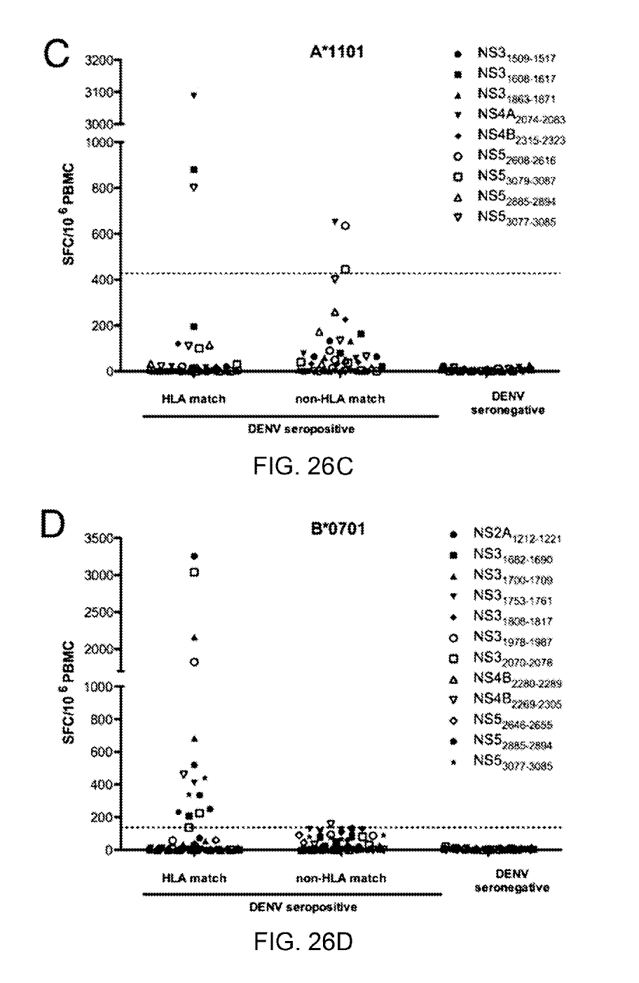

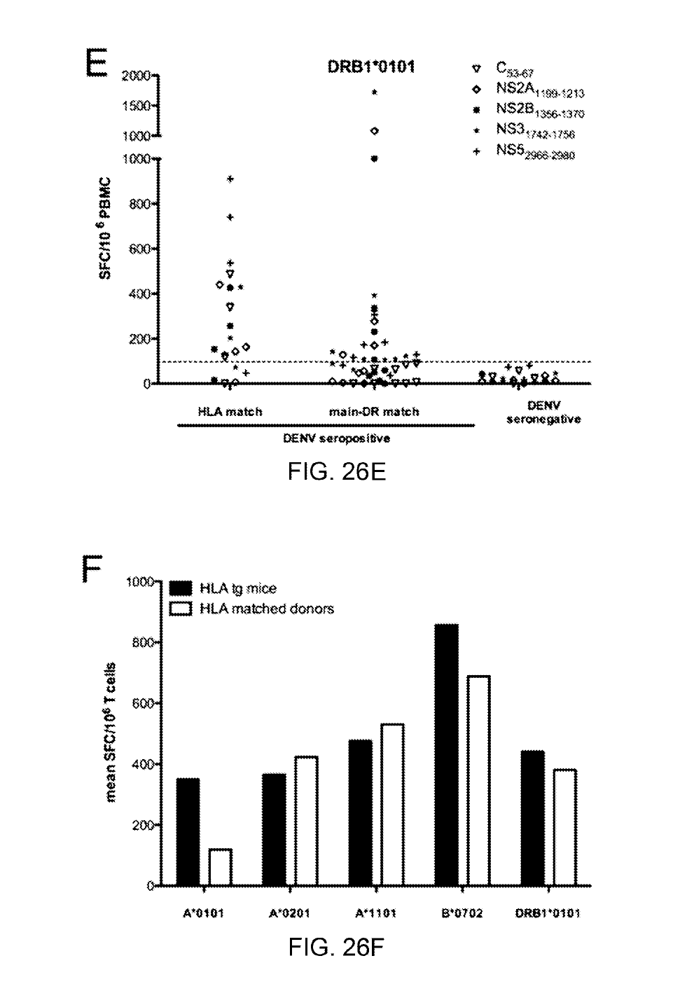

FIGS. 26A-26D (HLA A*0101, A*0201, A*1101, and B*0702) show the capacity of the identified epitopes to stimulate PBMC from the various donor categories. Each of the A*0101and A*0201 epitopes was detected at least once in an HLA matched donor, although the magnitude as well as the frequency of responses was higher for the A*0201 restricted epitopes (FIGS. 26A-26B and Table 2). Out of the 9 A*1101 restricted epitopes, 3 have been detected once in HLA matched donors. These three epitopes though have been able to stimulate a robust IFNγ response, as indicated by net SFCs>800 (FIG. 26C). In case of the B*0702 restricted epitopes, 10 out of the 12 have been detected in one or more HLA matched donors as shown in FIG. 26D and Table 1. No significant responses could be detected in non -HLA matched donors studied, as shown for A1, A2, A3 and B7 molecules. In contrast, all four restricted DRB1*0101 epitopes have been detected in 3 out of 4 HLA matched donors tested and were also able to elicit significant IFNγ responses in non-HLA matched donors. This is in accordance with recent reports, demonstrating a high degree of repertoire sharing across MHC class II molecules (Greenbaum, et al. Immunogenetics 63:325 (2011)). Overall, responses to 34 of the 42 epitopes were detected in at least one donor, which corresponds to an overlap of 81% between the murine and human system. In addition to the experimental approach, an IEDB query was performed with the epitopes identified in the mouse model. Here, 13 of the 42 epitopes previously described to elicit an IFNγ in DENV seropositive individuals were identified, as indicated in Table 2. The 30% overlap with known epitopes contributes to the validation of our mouse model and shows on the other hand that 70% of the epitopes identified are novel, contributing to an extended knowledge of T cell mediated responses to DENV.

### Example 20

This example includes studies showing dominance of B7 responses.

A notable observation here was that out of all HLA transgenic mouse strains tested the strongest CD8⁺ T cell responses could be detected in the B*0702 transgenic IFN-α/βR^{-/-} mice. Four B*0702 restricted epitopes were able to elicit an IFNγ response above a thousand SFC/10⁶ CD8⁺ T cells. On average B*0702 epitopes were able to elicit an IFNγ response of 857 SFC/10⁶ CD8⁺ T cells, compared to an average of 350, 365, and 476 SFC/10⁶ CD8⁺ T cells for the HLA A*0101, A*0201 and A*1101 restricted epitopes, respectively (FIG. 26F, black bars). Most interestingly, the exact same response pattern could be observed testing PBMC from HLA matched donors, previously exposed to DENV (FIG. 26F, white bars). As seen in mice, B*0702 restricted epitopes were able to elicit the strongest IFNγ responses, reaching an average of 688 SFC/10⁶ CD8⁺ T cells, followed by an average of 530, 423 and 119 SFC/10⁶ CD8⁺ T cells for HLA*1101, A*0202 and A*0101 restricted epitopes, respectively. The fact that the mouse model described herein reflects response patterns observed in humans makes it an especially suitable model to identify and study epitopes of human relevance to DENV infection.

### Example 21

This example includes a description of studies showing the subprotein location of identified epitopes, and the conservancy of identified epitopes within the DENV2 serotype.

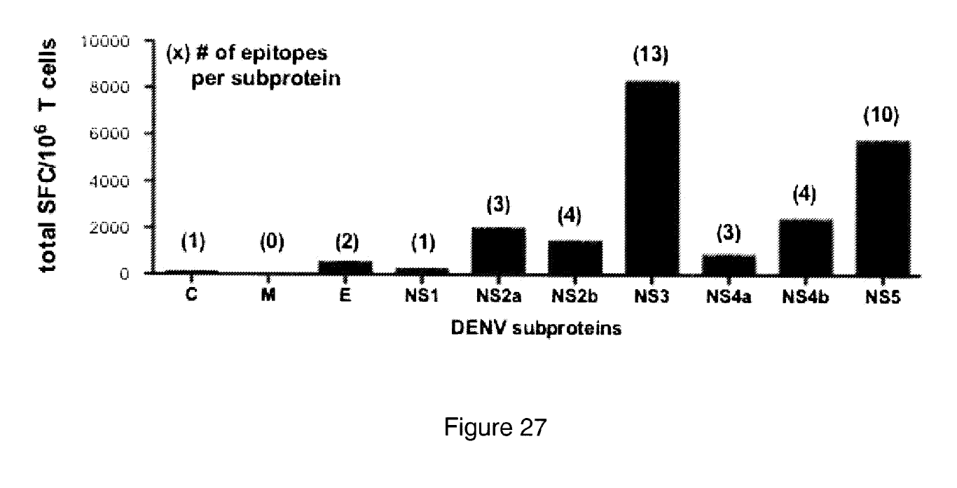

The identified epitopes are derived from 9 of the 10 DENV proteins, with the membrane protein being the only protein where no epitope could be detected (FIG. 27). The majority of epitopes are derived from the seven nonstructural proteins. 39 out of 42 of the identified epitopes (93%) originate from the nonstructural proteins, accounting for 97% of the total IFNγ response observed. Within the nonstructural proteins, however, NS3 and NS5 alone account for 67% of the total response, representing a total number of 23 epitopes detected from these two proteins. NS5 is furthermore the only subprotein where at least one derived epitope has been identified in all five HLA transgenic mouse strains. These results are consistent with the immunodominance of NS3, but also identify NS5 as a major target of T cell responses.

Cross-reactivity of T cells is a well-described phenomenon in DENV infection (Mathew, et al. Immunol Rev 225:300 (2008))). To circumvent this issue, T cell reactivity was exclusively tested to S221 derived peptides, which was also used as infectious agent in this study. However, to assess the relevance for infections with other DENV2 strains, conservancy of these epitopes within the DENV2 serotype was analyzed. 171 full-length DENV2 polyprotein sequences from the NCBI

Protein database were analyzed for conservancy. Of the epitopes identified, 30 out of the 42 epitopes were conserved in >90% of all DENV2 strains; 8 epitopes were even conserved in all 171 strains analyzed. Of the remaining 12 epitopes, 6 were conserved in >75% of all strains analyzed and the other half was found in the 30-65% range. This accounts for an average conservancy of 92% for the epitopes identified, which is significantly higher than the average conservancy of non-epitopes (73%; p<0.001).

To determine if the epitopes identified were also conserved in serotypes, other than DENV2, 162 DENV1, 169 DENV3 and 53 DENV4 sequences from the NCBI protein database were studied for conservancy. In contrast to a high degree of conservancy within the DENV2 serotype, 35 out of the 42 epitopes did not occur in any of the 384 DENV-1, 3 and 4 sequences tested and only 7 epitopes had sequence homologues in one or more of the other serotypes. Interestingly, most of the epitopes which show conservancy across serotypes have been identified in the B*0702 transgenic mice. 4 of the identified B*0702 restricted epitopes (NS3₁₆₈₂₋₁₆₉₀, NS3₁₇₀₀₋₁₇₀₉, NS3₁₇₅₃₋₁₇₆₁, NS4B₂₂₈₀₋₂₈₉₂) were additionally conserved in 89-100% of sequences derived from serotypes other than DENV2. The same has been observed for two DRB1*0101 restricted epitopes which were conserved across serotypes (NS3₁₇₄₂₋₁₇₅₆, NS5₂₉₆₆₋₂₉₈₀). Here, the epitopes were conserved in >99% of polyprotein sequences of two serotypes other than DENV2. Finally, one of the A*0101 restricted epitopes (NS1₁₀₉₀₋₁₀₉₉) is also conserved in 100% of DENV3 sequences. All results from this analysis are shown in Tables 2 and 3.

The DENV2 epitopes identified in Table 2 were analyzed for their respective homologues in DENV1, DENV3 and DENV4. 162 DENV1, 171 DENV2, 169 DENV3 and 53 DENV4 sequences from the NCBI Protein database were analyzed for conservancy. Table 3 shows the sequences of the epitopes identified after infection with DENV2 (bold letters). “Counts” indicate the number of strains in which the epitope is conserved within the respective serotype. Listed for each epitope are variants of the epitope in the DENV1, 3 and 4 serotypes and their respective counts. Epitopes are sorted according to their appearance in Table 2. These sequences help determine the cross-reactivity patterns of the identified epitopes.

TABLE 3
Conservancy and Variants of Epitopes
Identified - CD8 Epitopes

| Epitope | Sequence | Serotype | Counts |
|---|---|---|---|
| E₄₅₁₋₄₅₉ | ITEAELTGY | DENV2 | 146 |
| | STEIQLTDY | DENV1 | 5 |
| | TTEIQLTDY | DENV1 | 37 |
| | TSEIQLIDY | DENV1 | 1 |
| | TSEIQLTDY | DENV1 | 119 |
| | IAEAELTGY | DENV2 | 3 |
| | IAEAELTDY | DENV2 | 6 |
| | ITDAELTGY | DENV2 | 2 |
| | STEAELTGY | DENV2 | 2 |
| | TTEAELTGY | DENV2 | 10 |
| | ISEAELTDY | DENV2 | 2 |
| | ITEAELTGY | DENV2 | 146 |
| | TVEAVLLEY | DENV3 | 1 |
| | TVEAVLPEY | DENV3 | 40 |
| | TVEAILPEY | DENV3 | 44 |
| | TAEAILPEY | DENV3 | 4 |
| | THEALLPEY | DENV3 | 1 |
| | ITEAILPEY | DENV3 | 3 |
| | TTEVILPEY | DENV3 | 1 |
| | TTEAILPEY | DENV3 | 75 |
| | SVEVELPDY | DENV4 | 2 |
| | SVEVKLPDY | DENV4 | 51 |
| NS1₁₀₉₀₋₁₀₉₉ | RSCTLPPLRY | DENV2 | 171 |
| | RSCTLPPLRF | DENV1 | 162 |
| | RSCTLPPLRY | DENV2 | 171 |
| | RSCTLPPLRY | DENV3 | 169 |
| | RSCTMPPLRF | DENV4 | 53 |
| NS2A₁₁₉₂₋₁₂₀₀ | MTDDIGMGV | DENV2 | 143 |
| | ASDRMGMGM | DENV1 | 1 |
| | ASDMMGMGT | DENV1 | 2 |
| | ASDKMGMGT | DENV1 | 24 |
| | ASDNMGMGT | DENV1 | 11 |
| | VSDRMGMGT | DENV1 | 6 |
| | ASDRMGMGT | DENV1 | 118 |
| | MADDIGMGV | DENV2 | 12 |
| | MTDEMGMGV | DENV2 | 14 |
| | ITDDIGMGV | DENV2 | 2 |
| | MTDDIGMGV | DENV2 | 143 |
| | ASDRTGMGV | DENV3 | 1 |
| | ASDKMGMGV | DENV3 | 4 |
| | ATDRMGMGV | DENV3 | 1 |
| | ASDRMGMGV | DENV3 | 163 |
| NS2A₁₂₅₁₋₁₂₅₉ | LTDALALGM | DENV2 | 156 |
| | LGDGLAIGI | DENV1 | 1 |
| | LGDGFAMGI | DENV1 | 1 |
| | LGDGLAMGI | DENV1 | 160 |
| | LTDAIALGI | DENV2 | 13 |
| | LTDAWALGM | DENV2 | 1 |
| | LTDALALGI | DENV2 | 1 |
| | LTDALALGM | DENV2 | 156 |
| | MANGVALGL | DENV3 | 2 |
| | MANGIALGL | DENV3 | 167 |
| | LISGISLGL | DENV4 | 1 |
| | FIDGLSLGL | DENV4 | 1 |
| | LIDGISLGL | DENV4 | 45 |
| | LIDGIALGL | DENV4 | 1 |
| | FIDGISLGL | DENV4 | 5 |
| NS4B₂₃₉₉₋₂₄₀₇ | VIDLDPIPY | DENV2 | 90 |
| | TIDLDPVVY | DENV1 | 6 |
| | AIDLDPVVY | DENV1 | 156 |
| | VIDLEPIPY | DENV2 | 81 |
| | VIDLDPIPY | DENV2 | 90 |
| | TIDLDSVIF | DENV3 | 1 |
| | TIDLDPVIY | DENV3 | 167 |
| | TIALDPVIY | DENV3 | 1 |
| | VIDLEPISY | DENV4 | 53 |
| NS5₃₃₇₅₋₃₃₈₃ | YTDYMPSMK | DENV2 | 168 |
| | YSDYMTSMK | DENV1 | 8 |
| | YLDYMASMK | DENV1 | 1 |
| | YIDYMTSMK | DENV1 | 1 |
| | YLDFMTSMK | DENV1 | 6 |
| | YLDYMTSMK | DENV1 | 143 |
| | YLDYMISMK | DENV1 | 2 |
| | YIDYMPSMK | DENV2 | 1 |
| | YMDYMPSMK | DENV2 | 2 |
| | YTDYMPSMK | DENV2 | 168 |
| | FLDYMPSMK | DENV3 | 169 |
| | YADYMPVMK | DENV4 | 1 |
| | YMDYMPVMK | DENV4 | 1 |
| | YVDYMPAMK | DENV4 | 5 |
| | YVDYMPVMR | DENV4 | 2 |
| | YVDYMPVMK | DENV4 | 44 |
| E₆₃₁₋₆₃₉ | RLITVNPIV | DENV2 | 168 |
| | RVITANPIV | DENV1 | 7 |
| | RLVTANPIV | DENV1 | 11 |
| | RLITANPIV | DENV1 | 144 |
| | RLITVNPVV | DENV2 | 1 |
| | RLITVNPII | DENV2 | 1 |
| | RLITVNPIV | DENV2 | 168 |
| | RLTTVNPIV | DENV2 | 1 |
| | RLITANPIV | DENV3 | 11 |
| | RLITANPVV | DENV3 | 158 |
| | RVISATPLA | DENV4 | 11 |
| | RVISSTPLA | DENV4 | 15 |
| | RIISSTPLA | DENV4 | 9 |
| | RVISSTPFA | DENV4 | 1 |
| | RIISSTPFA | DENV4 | 16 |
| | RIISSIPFA | DENV4 | 1 |
| NS2B₁₃₅₅₋₁₃₆₃ | IMAVGMVSI | DENV2 | 157 |
| | IMAVGVVSI | DENV1 | 2 |
| | VMAVGIVSI | DENV1 | 1 |
| | IMAIGIVSI | DENV1 | 64 |
| | IMAVGIVSI | DENV1 | 95 |
| | VMAVGMVSI | DENV2 | 14 |
| | IMAVGMVSI | DENV2 | 157 |
| | VMAIGLVSI | DENV3 | 3 |
| | VMAVGLVSI | DENV3 | 166 |
| | MMAVGLVSL | DENV4 | 1 |
| | IMAVGLVSL | DENV4 | 52 |
| NS2B₁₃₈₃₋₁₃₉₁ | GLLTVCYVL | DENV2 | 170 |
| | GMLITCYVI | DENV1 | 1 |
| | GMLIACYVI | DENV1 | 161 |
| | GPLTVCYVL | DENV2 | 1 |
| | GLLTVCYVL | DENV2 | 170 |
| | GMLIACYVI | DENV3 | 2 |
| | GLLIACYVI | DENV3 | 167 |
| | GLLLAAYMM | DENV4 | 1 |
| | GLLLAAYVM | DENV4 | 52 |
| NS4A₂₀₇₄₋₂₀₈₃ | RIYSDPLALK | DENV2 | 153 |
| | RTYSDPQALR | DENV1 | 1 |
| | RTYSDPLALR | DENV1 | 161 |
| | RTYSDPLALK | DENV2 | 13 |
| | RIYSDPLTLK | DENV2 | 2 |
| | KIYSDPLALK | DENV2 | 2 |
| | RIYSEPRALK | DENV2 | 1 |
| | RIYSDPLALK | DENV2 | 153 |
| | RTYSDPLAPK | DENV3 | 1 |
| | RTYSDPLALK | DENV3 | 167 |
| | RIYSDPLALK | DENV3 | 1 |
| | RVYADPMALQ | DENV4 | 1 |
| | RVYADPMALK | DENV4 | 52 |
| NS4B₂₃₁₅₋₂₃₂₃ | ATVLMGLGK | DENV2 | 168 |
| | AAILMGLDK | DENV1 | 162 |
| | ATVLMGLGK | DENV2 | 168 |
| | ATVLMGLGR | DENV2 | 3 |
| | AVVLMGLNK | DENV3 | 1 |
| | AVVLMGLDK | DENV3 | 168 |
| | AAVLMGLGK | DENV4 | 53 |
| NS5₂₆₀₈₋₂₆₁₆ | STYGWNLVR | DENV2 | 171 |
| | AAYGWNLVK | DENV1 | 1 |
| | ATYGWNLVK | DENV1 | 161 |
| | STYGWNLVR | DENV2 | 171 |
| | STYGWNLVK | DENV3 | 3 |
| | STYGWNVVK | DENV3 | 1 |
| | STYGWNIVK | DENV3 | 165 |
| | ATYGWNLVK | DENV4 | 53 |
| NS5₃₀₇₉₋₃₀₈₇ | TVMDIISRR | DENV2 | 155 |
| | TVMDIISRR | DENV1 | 1 |
| | TVMDVISRR | DENV1 | 161 |
| | TVLDIISRR | DENV2 | 1 |
| | TVMDIISRK | DENV2 | 15 |
| | TVMDIISRR | DENV2 | 155 |
| | TVMDIISRK | DENV3 | 169 |
| | AVMDIISRK | DENV4 | 53 |
| NS5₃₁₁₂₋₃₂₉₁ | RQMEGEGVFK | DENV2 | 74 |
| | RQMESEEIFS | DENV1 | 1 |
| | RQMESEGIVS | DENV1 | 1 |
| | RQMESEGIFF | DENV1 | 5 |
| | RQMESEGIIL | DENV1 | 1 |
| | RQMESEGIFS | DENV1 | 87 |
| | RQMESEGIFL | DENV1 | 67 |
| | RQMEGEGVFR | DENV2 | 1 |
| | RQMEGEGIFR | DENV2 | 1 |
| | RQMEGEGLFK | DENV2 | 13 |
| | RQMEGEEVFK | DENV2 | 1 |
| | RQMEGEGVFK | DENV2 | 74 |
| | RQMEGEGIFK | DENV2 | 81 |
| | RQMEGEGVLT | DENV3 | 12 |
| | RQMEGEGVLS | DENV3 | 155 |
| | RQMEGEDVLS | DENV3 | 2 |
| | RQMEAEGVIT | DENV4 | 53 |
| NS5₃₂₈₃₋₃₂₉₁ | RTTWSIHAK | DENV2 | 111 |
| | RTTWSIHAH | DENV1 | 162 |
| | RTTWSIHAR | DENV2 | 8 |
| | RTTWSIHAT | DENV2 | 31 |
| | RTTWSIHAS | DENV2 | 21 |
| | RTTWSIHAK | DENV2 | 111 |
| | RTTWSIHAH | DENV3 | 169 |
| | RTTWSIHAH | DENV4 | 53 |
| NS2A₁₂₁₂₋₁₂₂₁ | RPTFAAGLLL | DENV2 | 158 |
| | RPMLAVGLLF | DENV1 | 1 |
| | RPMFAMGLLF | DENV1 | 1 |
| | RPMFAVGLLI | DENV1 | 4 |
| | RPMFAVGLLF | DENV1 | 156 |
| | RPTFAAGLFL | DENV2 | 1 |
| | RPTFAVGLVL | DENV2 | 1 |
| | RPTFAVGLLL | DENV2 | 11 |
| | RPTFAAGLLL | DENV2 | 158 |
| | QPFLALGFFM | DENV3 | 1 |
| | QPFLTLGFFL | DENV3 | 1 |
| | QPFLALGFFL | DENV3 | 167 |
| | SPRYVLGVFL | DENV4 | 1 |
| | SPGYVLGVFL | DENV4 | 46 |
| | SPGYVLGIFL | DENV4 | 6 |
| NS3₁₆₈₂₋₁₆₉₀ | LPAIVREAI | DENV2 | 171 |
| | LPAIIREAI | DENV1 | 1 |
| | LPAIVREAI | DENV1 | 158 |
| | LPAMVREAI | DENV1 | 3 |
| | LPAIVREAI | DENV2 | 171 |
| | LPTIVREAI | DENV3 | 2 |
| | LPAVVREAI | DENV3 | 1 |
| | LPAIVREAI | DENV3 | 163 |
| | LPAIIREAI | DENV3 | 3 |
| | LPSIVREAL | DENV4 | 53 |
| NS3₁₇₀₀₋₁₇₀₉ | APTRVVAAEM | DENV2 | 170 |
| | APTRVVASET | DENV1 | 1 |
| | APTRVVAAEM | DENV1 | 1 |
| | APTRVVASEM | DENV1 | 160 |
| | APPRVVPAEM | DENV2 | 1 |
| | APTRVVAAEM | DENV2 | 170 |
| | APTRVVAAEM | DENV3 | 169 |
| | APTRVVAAEM | DENV4 | 53 |
| NS3₁₇₅₃₋₁₇₆₁ | VPNYNLIIM | DENV2 | 171 |
| | VPNYNMIIV | DENV1 | 1 |
| | VPNYNMIIM | DENV1 | 160 |
| | VPNYNMIVM | DENV1 | 1 |
| | VPNYNLIIM | DENV2 | 171 |
| | VPNYNLIVM | DENV3 | 11 |
| | VPNYNLVVM | DENV3 | 1 |
| | VPNYNLVIM | DENV3 | 6 |
| | VSNYNLIIM | DENV3 | 1 |
| | VPNYNLIIM | DENV3 | 150 |
| | VPNYNLIVM | DENV4 | 53 |
| NS3₁₈₀₈₋₁₈₁₇ | APIMDEEREI | DENV2 | 131 |
| | AIIQDEERDI | DENV1 | 1 |
| | AVIQDEEKDI | DENV1 | 13 |
| | AAIQDEERDI | DENV1 | 3 |
| | AVIQDEERDI | DENV1 | 145 |
| | APIMDDEREI | DENV2 | 1 |
| | APIIDEEREI | DENV2 | 30 |
| | APIVDEEREI | DENV2 | 9 |
| | APIMDEEREI | DENV2 | 131 |
| | APIQDEEKDI | DENV3 | 2 |
| | SPIQDEERDI | DENV3 | 1 |
| | APIQDEERDI | DENV3 | 164 |
| | APIQDKERDI | DENV3 | 2 |
| | SPIEDIEREI | DENV4 | 53 |
| NS3₁₉₇₈₋₁₉₈₇ | TPEGIIPSMF | DENV2 | 170 |
| | TPEGIIPALY | DENV1 | 1 |
| | TPEGIIPALF | DENV1 | 161 |
| | TPEGIIPSLF | DENV2 | 1 |
| | TPEGIIPSMF | DENV2 | 170 |
| | TPEGIIPALF | DENV3 | 169 |
| | TPEGIIPTLF | DENV4 | 53 |
| NS5₂₉₆₆₋₂₉₈₀ | SRAIWYMWLGARFLE | DENV2 | 171 |
| | SRAIWYVWLGARFLE | DENV1 | 1 |
| | SRAIWYMWLGAAFLE | DENV1 | 1 |
| | SRAIWYMWLGARFLE | DENV1 | 160 |
| | SRAIWYMWLGARFLE | DENV2 | 171 |
| | SRAIWYMWLGARFLE | DENV3 | 5 |
| | SRAIWYMWLGVRYLE | DENV3 | 1 |
| | SRAIWYMWLGARYLE | DENV3 | 163 |
| | SRAIWYMWLGARFLE | DENV4 | 53 |
| NS2B₁₃₈₃₋₁₃₉₁ | LLVISGLFPV | DENV2 | 85 |
| | LLAISGVYPL | DENV1 | 1 |
| | LLAVSGMYPL | DENV1 | 5 |
| | LLAVSGVYPL | DENV1 | 49 |
| | LLVISGVYPM | DENV1 | 1 |
| | LLAVSGVYPI | DENV1 | 2 |
| | LLAASGVYPM | DENV1 | 1 |
| | LLAISGVYPM | DENV1 | 27 |
| | LLAVSGVYPM | DENV1 | 76 |
| | LLVVSGLFPV | DENV2 | 1 |
| | LLVISGLFPA | DENV2 | 1 |
| | LLVISGLFPI | DENV2 | 15 |
| | LLVISGVFPV | DENV2 | 69 |
| | LLVISGLFPV | DENV2 | 85 |
| | LLIVSGIFPC | DENV3 | 1 |
| | LLIVSGIFPY | DENV3 | 151 |
| | LLIVSGVFPY | DENV3 | 17 |
| | LITVSGLYPL | DENV4 | 53 |
| NS3₁₄₆₅₋₁₄₇₃ | AAAWYLWEV | DENV2 | 157 |
| | LFVWCFWQK | DENV1 | 1 |
| | LFLWYFWQK | DENV1 | 1 |
| | LFVWHFWQK | DENV1 | 6 |
| | FFVWYFWQK | DENV1 | 1 |
| | PFVWYFWQK | DENV1 | 1 |
| | LFVWYFWQK | DENV1 | 152 |
| | AAAWYLWET | DENV2 | 13 |
| | AAAWYLWEA | DENV2 | 1 |
| | AAAWYLWEV | DENV2 | 157 |
| | LLVWHAWQK | DENV3 | 1 |
| | MLVWHTWQK | DENV3 | 1 |
| | LLVWHTWQK | DENV3 | 167 |
| | MALWYIWQV | DENV4 | 9 |
| | MTLWYMWQV | DENV4 | 42 |
| | MALWYMWQV | DENV4 | 2 |
| NS3₁₆₈₁₋₁₆₈₉ | YLPAIVREA | DENV2 | 170 |
| | YLPAIIREA | DENV1 | 1 |
| | YLPAIVREA | DENV1 | 158 |
| | YLPAMVREA | DENV1 | 3 |
| | SLPAIVREA | DENV2 | 1 |
| | YLPAIVREA | DENV2 | 170 |
| | YLPTIVREA | DENV3 | 2 |
| | YLPAVVREA | DENV3 | 1 |
| | YLPAIVREA | DENV3 | 163 |
| | YLPAIIREA | DENV3 | 3 |
| | ILPSIVREA | DENV4 | 53 |
| NS3₂₀₁₃₋₂₀₂₂ | DLMRRGDLPV | DENV2 | 157 |
| | DLLRRGDLPV | DENV1 | 1 |
| | ELMRRGDLPV | DENV1 | 161 |
| | DLMKRGDLPV | DENV2 | 11 |
| | ELMRRGDLPV | DENV2 | 3 |
| | DLMRRGDLPV | DENV2 | 157 |
| | ELMRRGHLPV | DENV3 | 2 |
| | ELMRRGDLPV | DENV3 | 167 |
| | ELMKRGDLPV | DENV4 | 2 |
| | ELMRRGDLPV | DENV4 | 51 |
| NS4A₂₁₄₀₋₂₁₄₈ | ALSELPETL | DENV2 | 169 |
| | ALEELPDTI | DENV1 | 5 |
| | AVEELPDTI | DENV1 | 1 |
| | AMEELPDTI | DENV1 | 156 |
| | ALSELAETL | DENV2 | 1 |
| | ALGELPETL | DENV2 | 1 |
| | ALSELPETL | DENV2 | 169 |
| | AVEELPETM | DENV3 | 169 |
| | ALNELTESL | DENV4 | 1 |
| | ALNELPESL | DENV4 | 52 |
| NS4A₂₂₀₅₋₂₂₁₃ | IILEFFLIV | DENV2 | 170 |
| | IILKFFLMV | DENV1 | 1 |
| | IILEFLLMV | DENV1 | 1 |
| | IMLEFFLMV | DENV1 | 1 |
| | IILEFFLMV | DENV1 | 159 |
| | IILEFFLMV | DENV2 | 1 |
| | IILEFFLIV | DENV2 | 170 |
| | IILEFFMMV | DENV3 | 1 |
| | IVLEFFMMV | DENV3 | 168 |
| | IILEFFLMV | DENV4 | 53 |
| NS5₃₀₅₈₋₃₀₆₆ | KLAEAIFKL | DENV2 | 162 |
| | LLAKAIFKL | DENV1 | 15 |
| | QLAKSIFKL | DENV1 | 1 |
| | LLATSVFKL | DENV1 | 1 |
| | LLAKSIFKL | DENV1 | 26 |
| | LLATAIFKL | DENV1 | 1 |
| | LLATSIFKL | DENV1 | 117 |
| | LLASSIFKL | DENV1 | 1 |
| | KLAEAIFRL | DENV2 | 6 |
| | RLAEAIFKL | DENV2 | 2 |
| | KLAEAVFKL | DENV2 | 1 |
| | KLAEAIFKL | DENV2 | 162 |
| | QLASAIFKL | DENV3 | 6 |
| | LLANAIFKL | DENV3 | 1 |
| | RLANAIFKL | DENV3 | 2 |
| | QLANAIFKL | DENV3 | 160 |
| | TLAKAIFKL | DENV4 | 9 |
| | ILAKAIFKL | DENV4 | 44 |
| NS3₁₅₀₉₋₁₅₁₇ | SQIGAGVYK | DENV2 | 168 |
| | SQVGVGVFQ | DENV1 | 162 |
| | SQIGAGVYR | DENV2 | 1 |
| | SQIGTGVYK | DENV2 | 1 |
| | SQIGVGVYK | DENV2 | 1 |
| | SQIGAGVYK | DENV2 | 168 |
| | TQVGVGIQK | DENV3 | 3 |
| | TQVGVGVHK | DENV3 | 2 |
| | TQVGVGVQK | DENV3 | 164 |
| | TQVGVGIHI | DENV4 | 4 |
| | TQVGVGIHT | DENV4 | 1 |
| | TQVGVGIHM | DENV4 | 47 |
| | TQVGVGVHV | DENV4 | 1 |
| NS3₁₆₀₈₋₁₆₁₇ | GTSGSPIIDK | DENV2 | 49 |
| | GTSGSPIVSR | DENV1 | 1 |
| | GTSGSPIVNR | DENV1 | 161 |
| | GTSGSPIIDK | DENV2 | 49 |
| | GTSGSPIADK | DENV2 | 1 |
| | GTSGSPIVDR | DENV2 | 75 |
| | GTSGSPIVDK | DENV2 | 46 |
| | GTSGSPIINK | DENV3 | 1 |
| | GTSGSPIINR | DENV3 | 168 |
| | GSSGSPIINR | DENV4 | 1 |
| | GTSGSPIVNR | DENV4 | 1 |
| | GTSGSPIINK | DENV4 | 13 |
| | GTSGSPIINR | DENV4 | 38 |
| NS3₁₈₆₃₋₁₈₇₁ | KTFDSEYVK | DENV2 | 129 |
| | KTFDTEYQK | DENV1 | 162 |
| | KTFDTEYTK | DENV2 | 5 |
| | KTFDTEYIK | DENV2 | 7 |
| | KTFDFEYIK | DENV2 | 1 |
| | KTFDSEYIK | DENV2 | 26 |
| | KTFDSEYAK | DENV2 | 3 |
| | KTFDSEYVK | DENV2 | 129 |
| | KTFDTEYQR | DENV3 | 1 |
| | KTFNTEYQK | DENV3 | 1 |
| | KTFDTEYQK | DENV3 | 167 |
| | KTFDTEYPK | DENV4 | 53 |
| NS3₂₀₇₀₋₂₀₇₈ | KPRWLDARI | DENV2 | 155 |
| | RPRWLDART | DENV1 | 162 |
| | KPRWLDART | DENV2 | 13 |
| | KPRWLDAKI | DENV2 | 2 |
| | KPRWLDPRI | DENV2 | 1 |
| | KPRWLDARI | DENV2 | 155 |
| | RPRWLDART | DENV3 | 168 |
| | RPRWLDARI | DENV3 | 1 |
| | RPRWLDARV | DENV4 | 24 |
| | RPKWLDARV | DENV4 | 29 |
| NS4B₂₂₈₀₋₂₂₈₉ | RPASAWTLYA | DENV2 | 171 |
| | HPASAWTLYA | DENV1 | 102 |
| | RPASAWTLYA | DENV1 | 60 |
| | RPASAWTLYA | DENV2 | 171 |
| | HPASAWILYA | DENV3 | 1 |
| | HPASAWTLYA | DENV3 | 168 |
| | RPASAWTLYA | DENV4 | 53 |
| NS4B₂₂₉₆₋₂₃₀₃ | TPMLRHSI | DENV2 | 171 |
| | TPMLRHTI | DENV1 | 1 |
| | TPMMRHTI | DENV1 | 161 |
| | TPMLRHSI | DENV2 | 171 |
| | TPMLRHTI | DENV3 | 169 |
| | TPMLRHTI | DENV4 | 53 |
| NS5₂₆₄₆₋₂₆₅₅ | SPNPTVEAGR | DENV2 | 92 |
| | SPNPTIEEGR | DENV1 | 162 |
| | SPSPTVEAGR | DENV2 | 1 |
| | SPNPTVDAGR | DENV2 | 1 |
| | SPNPTVEAGP | DENV2 | 1 |
| | SPNPTIEAGR | DENV2 | 76 |
| | SPNPTVEAGR | DENV2 | 92 |
| | SPSPTVEEGR | DENV3 | 1 |
| | SPSLTVEESR | DENV3 | 1 |
| | SPSPIVEESR | DENV3 | 1 |
| | SPSPTVEESR | DENV3 | 166 |
| | SSNPTIEEGR | DENV4 | 53 |
| NS5₂₈₈₅₋₂₈₉₄ | TPRMCTREEF | DENV2 | 152 |
| | KPRICTREEF | DENV1 | 162 |
| | RPRICTRAEF | DENV2 | 1 |
| | KPRICTRAEF | DENV2 | 12 |
| | TRRMCTREEF | DENV2 | 1 |
| | TPRICTREEF | DENV2 | 3 |
| | IPRMCTREEF | DENV2 | 2 |
| | TPRMCTREEF | DENV2 | 152 |
| | KPRLCPREEF | DENV3 | 1 |
| | KPRLCTREEF | DENV3 | 88 |
| | RPRLCTREEF | DENV3 | 80 |
| | NPRLCTKEEF | DENV4 | 1 |
| | SPRLCTREEF | DENV4 | 6 |
| | TPRLCTREEF | DENV4 | 2 |
| | SPRLCTKEEF | DENV4 | 2 |
| | NPRLCTREEF | DENV4 | 41 |
| | KPRLCTREEF | DENV4 | 1 |
| NS5₃₀₇₇₋₃₀₈₅ | RPTPRGTVM | DENV2 | 166 |
| | RPVKNGTVM | DENV1 | 1 |
| | RPARNGTVM | DENV1 | 1 |
| | RPAKNGTVM | DENV1 | 147 |
| | RPAKSGTVM | DENV1 | 13 |
| | RPTPRGTVL | DENV2 | 1 |
| | RPTPKGTVM | DENV2 | 2 |
| | RPTPIGTVM | DENV2 | 2 |
| | RPTPRGTVM | DENV2 | 166 |
| | RPTPKGTVM | DENV3 | 89 |
| | RPTPTGTVM | DENV3 | 80 |
| | RPTPRGAVM | DENV4 | 35 |
| | RPTPKGAVM | DENV4 | 18 |
| C₅₃₋₆₇ | AFLRFLTIPPTAGIL | DENV2 | 169 |
| | AFLRFLAIPPTAGIV | DENV1 | 1 |
| | ALLRFLAIPPTAGIL | DENV1 | 2 |
| | AFLTFLAIPPTAGIL | DENV1 | 1 |
| | AFLRFLAIPPTAGIL | DENV1 | 158 |
| | AFLRFLTISPTAGIL | DENV2 | 1 |
| | AFLRFLTIPPTVGIL | DENV2 | 1 |
| | AFLRFLTIPPTAGIL | DENV2 | 169 |
| | AFLRFLAIPPTAGIL | DENV3 | 20 |
| | AFLRFLAIPPTAGVL | DENV3 | 149 |
| | TFLRVLSIPPTAGIL | DENV4 | 53 |
| NS2A₁₁₉₉₋₁₂₁₃ | GVTYLALLAAFKVRP | DENV2 | 156 |
| | GTTYLALMATFRMRP | DENV1 | 27 |
| | GMTYLALMATFKMRP | DENV1 | 1 |
| | GTTYLALMATLKMRP | DENV1 | 1 |
| | GTTHLALMATFKMRP | DENV1 | 2 |
| | GTTYLALMATFKMRP | DENV1 | 131 |
| | GVTYLALLATFKVRP | DENV2 | 1 |
| | GVTYLALLAAYKVRP | DENV2 | 2 |
| | GVTYLALLAAFRVRP | DENV2 | 12 |
| | GVTYLALLAAFKVRP | DENV2 | 156 |
| | GVTYLALIATFEIQP | DENV3 | 1 |
| | GVTCLALIATFKIQP | DENV3 | 1 |
| | GVTYLALIATFKVQP | DENV3 | 1 |
| | GVTYLALIATFKIQP | DENV3 | 166 |
| | GQTHLAIMAVFKMSP | DENV4 | 23 |
| | GQIHLAIMAVFKMSP | DENV4 | 24 |
| | GQTHLAIMIVFKMSP | DENV4 | 2 |
| | GQVHLAIMAVFKMSP | DENV4 | 3 |
| | GQIHLAIMTMFKMSP | DENV4 | 1 |
| NS3₁₃₅₆₋₁₃₇₀ | MAVGMVSILASSLLK | DENV2 | 171 |
| | MAVGVVSILLSSLLK | DENV1 | 2 |
| | MAIGIVSILLSSLLK | DENV1 | 64 |
| | MAVGIVSILLSSLLK | DENV1 | 96 |
| | MAVGMVSILASSLLK | DENV2 | 171 |
| | MAVGLVSILASSFLR | DENV3 | 11 |
| | MAIGLVSILASSLLR | DENV3 | 3 |
| | MAVGLVSILASSLLR | DENV3 | 155 |
| | MAVGLVSLLGSALLK | DENV4 | 53 |
| NS3₁₇₄₂₋₁₇₅₆ | TFTMRLLSPVRVPNY | DENV2 | 120 |
| | TFTMRLLSPVRVPNY | DENV1 | 162 |
| | PFTMRLLSPVRVPNY | DENV2 | 1 |
| | TFTMRLLSPIRVPNY | DENV2 | 50 |
| | TFTMRLLSPVRVPNY | DENV2 | 120 |
| | TFTMRLLSPVRVSNY | DENV3 | 1 |
| | PFTMRLLSPVRVPNY | DENV3 | 1 |
| | TFTMRLLSPVRVPNY | DENV3 | 167 |
| | TFTTKLLSSTRVPNY | DENV4 | 1 |
| | TFTTRLLSSTRVPNY | DENV4 | 52 |

### Example 22

This example includes a discussion of the foregoing data and conclusions based upon the data.

Wild-type mice are resistant to DENV-induced disease, and therefore, development of mouse models for DENV infection to date has been challenging and has had to rely on infection of immunocompromised mice, non-physiologic routes of infection, and mouse-human chimeras (Yauch, et al. Antiviral Res 80:87 (2008)). Due to the importance of the IFN system in the host antiviral response, mice lacking the IFNR-α/β support a productive infection. A mouse-passaged DENV2 strain, S221, is highly immunogenic and also replicates to high levels in IFNR-α/β−/− mice, thus allowing the study of CD4⁺ and CD8⁺ T cell responses in DENV infection. In this murine model, vaccination with T cell epitopes prior to S221 infection provided significant protection (Yauch, et al. J Immunol 185:5405 (2010); Yauch, et al. J Immunol 182:4865 (2009)). While significant differences exist between human and murine TCR repertoires and processing pathways, HLA transgenic mice are fairly accurate models of human immune responses, especially when peptide immunizations are utilized. Numerous studies to date show that these mice develop T cell responses that mirror the HLA restricted responses observed in humans in context of various pathogens (Gianfrani, et al. Hum Immunol 61:438 (2000); Wentworth, et al. Eur J Immunol 26:97 (1996); Shirai, et al. J Immunol 154:2733 (1995); Ressing, et al. J Immunol 154:5934 (1995); Vitiello, et al. J Exp Med 173:1007 (1991); Diamond, et al. Blood 90:1751 (1997); Firat, et al. Eur J Immunol 29:3112 (1999); Le, et al. J Immunol 142:1366 (1989); Man, et al. Int Immunol 7:597 (1995)).

The data disclosed herein demonstrate that HLA transgenic IFNRα/βR^{-/-} mice are a valuable model to identify DENV epitopes recognized in humans. Not only were a number of HLA-restricted T cell responses identified, but the genome wide screen provided further insight into the subproteins targeted by T cells during DENV infection. The majority of DENV responses (97%) were derived from the nonstructural proteins; more than half of the epitopes identified originate from the NS3 and NS5 protein. The data show the immunodominant role of the highly conserved NS3 protein (Rothman Adv Virus Res 60:397 (2003); Duangchinda, et al. Proc Natl Acad Sci USA 107:16922 (2010)), and also suggest NS5 as a major target of T cell responses. Interestingly, proteins previously described as antibody targets (prM, E and NS1) (Rothman J Clin Invest 113:946 (2004)) accounted for less than 5% of all responses, with only 3 epitopes identified from these proteins. The observation that T cell and B cell epitopes after primary DENV infection are not derived from the same proteins may factor in vaccine design, since immunizing with NS3 and NS5 T cell epitopes would induce a robust T cell response without the risk of antibody-dependent-enhancement (ADE).

Another unique challenge in vaccine development is the high degree of sequence variation in a pathogen, characteristically associated with RNA viruses. This is of particular relevance in the case of DENV infections, where it is well documented that prior exposure to a different serotype may lead to more severe disease and immunopathology (Sangkawibha, et al. Am J Epidemiol 120:653 (1984)). The fact that there is also significant genetic variation within each serotype adds to the complexity of successful vaccinations (Twiddy, et al. Virology 298:63 (2002); Holmes, et al. Trends Microbiol 8:74 (2000)). It is hypothesized that in certain cases, peptide variants derived from the original antigen in the primary infection, with substitutions at particular residues, can induce a response that is qualitatively different from the response induced by the original antigen (for example inducing a different pattern of lymphokine production; Partial agonism), or even actively suppressing the response (TCR antagonism). Variants associated with this phenotype are often collectively referred to as Altered Peptide Ligands (APLs) (Yachi, et al. Immunity 25:203 (2006)). During secondary infections, the T cell response directed at the APL may lead to altered or aberrant patterns of lymphokine production, and TCR antagonist mediated inhibition of T cell responses (Kast, et al. J Immunol 152:3904 (1994)). Therefore, immunity to all four serotypes would provide an optimal DENV vaccine. It is generally recognized that conserved protein sequences represent important functional domains (Valdar Proteins 48:227 (2002)), thus mutations at these important protein sites could be detrimental to the survival of the virus. T cell epitopes that target highly conserved regions of a protein are therefore likely to target the majority of genetic variants of a pathogen (Khan, et al. Cell Immunol 244:141 (2006)). Most interestingly in this context was that epitopes that are highly conserved within the DENV2 serotype are the major target for T cells. This data suggests, that immunizations with peptides from a given serotype would protect from the majority of genotypes within this serotype. In contrast, the DENV2 derived epitopes identified are not conserved in other serotypes. These findings point to an immunization strategy with a collection of multiple non-crossreactive epitopes derived from each of the major DENV serotypes. The induction of separate non-crossreactive responses would avoid issues arising from incomplete crossreaction and APL/TCR antagonism effects.

In addition to sequence variation, HLA polymorphism adds to the complexity of studying T cell responses to DENV. MHC molecules are extremely polymorphic, with several hundred different variants known in humans (Klein, Natural History of the Major Histocompatibility Complex (1986); Hughes, et al. Nature 355:402 (1992)). Therefore, selecting multiple peptides matching different MHC binding specificities will increase coverage of the patient population for diagnostic and vaccine applications alike. However, different MHC types are expressed at dramatically different frequencies in different ethnicities. To address this issue, IFNR-α/βR−/− mice were backcrossed with mice transgenic for HLA A*0101, A*0201, A*1101, B*0702 and DRB1*0101. These four MHC class I alleles were chosen as representatives of four supertypes (A1, A2, A3 and B7, respectively) and allow a combined coverage of approximately 90% of the worldwide human population (Sette, et al. Immunogenetics 50:201 (1999)), with more than 50% expressing the specific alleles. HLA supertypes are not limited to class I molecules. Several studies have demonstrated the existence of HLA class II supertypes (Doolan, et al. J Immunol 165:1123 (2000); Wilson, et al. J Virol 75:4195 (2001); Southwood, et al. J Immunol 160:3363 (1998)) and functional classification has revealed a surprising degree of repertoire sharing across supertypes (Greenbaum, et al. Immunogenetics 63:325 (2011)). This is in accordance with the data, since the DRB1*0101 restricted epitopes were identified in almost every donor, regardless if the donor was expressing the actual allele. Overall, the mouse model significantly reflects the response pattern observed in humans and that HLA B restricted responses seem to be dominant in B*0702 transgenic mice as well as in human donors, expressing the B*0702 allele (FIG. 26F).

The dominance of HLA B responses has been shown in context of several other viruses, such as HIV, EBV, CMV, and Influenza (Kiepiela, et al. Nature 432:769 (2004); Bihl, et al. J Immunol 176:4094 (2006); Boon, et al. J Immunol 172:4435 (2004); Lacey, et al. Hum Immunol 64:440 (2003)), suggesting that this observation is not limited to RNA viruses, and in fact, it has even been described for an intracellular bacterial pathogen, Mycobacterium Tuberculosis (Lewinsohn, et al. PLoS Pathog 3:1240 (2007); Axelsson-Robertson, et al. Immunology 129:496 (2010)). Furthermore, HLA B restricted T cell responses have been described to be of higher magnitude (Bihl, et al. J Immunol 176:4094 (2006)) and to influence infectious disease course and outcome. In case of DENV, one particular B07 epitope was reported to elicit higher responses in patients with DHF compared to patients suffering from DF only and could therefore be associated with disease outcome (Zivna, et al. J Immunol 168:5959 (2002)). Other reports suggest a role for HLA B44, B62, B76 and B77 alleles in protection against developing clinical disease after secondary DENV infection, whereas other alleles were associated with contribution to pathology (Stephens, et al. Tissue Antigens 60:309 (2002); Appanna, et al. PLoS One 5 (2010). Accordingly, HLA alleles appear to be associated with clinical outcome of exposure to dengue virus, in previously exposed and immunologically primed individuals. The fact that the stronger B*07 response occurs in our human samples as well as in our mouse model of DENV infection validates the relevance of this mouse model, since it even mimics patterns of immuno-dominance observed in humans.

### Example 23

This example includes a description of the identification of T cell responses against additional DENV-derived peptides in human donors.

Peripheral blood samples were obtained from healthy adult blood donors from the National Blood Center in Colombo, Sri Lanka. DENV-seropositivity was determined by ELISA. Those samples that are positive for DENV-specific IgM or IgG are further examined by the FACS based neutralization assay to determine whether the donor may have been exposed to single or multiple DENV serotypes. For MHC class I binding predictions all 9- and 10-mer peptides were predicted for their binding affinity to their respective alleles. Binding predictions were performed using the command-line version of the consensus prediction tool available on the IEDB web site. Peptides were selected if they were in the top 1% of binders.

As HLA typing and ELISA results were available, donor samples were tested such that predicted peptides for all four serotypes were tested against all appropriate and available HLA types expressed by the donor. DENV specific T cell responses were detected directly ex vivo from our Sri Lankan donor cohort, as measured by an IFNγELISPOT assay. All epitopes that have been identified in one or more donors are listed in Table 4.

TABLE 4
Human Donor Table and DENV Epitopes

| | Protein location | | | | | | | T cell | HLA- |
|---|---|---|---|---|---|---|---|---|---|
| | Start | End | | Super- | | | Sero- | response | Binding |
| # | position | position | Sequence | type | Allele | Length | type | [SFC] | [IC50] |
| 1 | 43 | 51 | GPMKLVMAF | B7 | B*0702 | 9 | DENV1 | 32 | 13 |
| 2 | 43 | 52 | GPMKLVMAFI | B7 | B*0702 | 10 | DENV1 | 62 | 86 |
| 3 | 49 | 57 | MAFIAFLRF | B7 | B*3501 | 9 | DENV1 | 82 | 3 |
| 4 | 75 | 83 | KSGAIKVLK | A3 | A*1101 | 9 | DENV3 | 823 | 151 |
| 5 | 104 | 113 | ITLLCLIPTV | A2 | A*0201 | 10 | DENV4 | 43 | 441 |
| 6 | 105 | 114 | CLMMMLPATL | A2 | A*0201 | 10 | DENV3 | 63 | 26 |
| 7 | 105 | 113 | TLLCLIPTV | A2 | A*0201 | 9 | DENV4 | 42 | 1 |
| 8 | 106 | 114 | LMMMLPATL | A2 | A*0201 | 9 | DENV3 | 78 | 22 |
| 9 | 106 | 115 | LMMMLPATLA | A2 | A*0201 | 10 | DENV3 | 50 | 14 |
| 10 | 107 | 115 | MMMLPATLA | A2 | A*0201 | 9 | DENV3 | 62 | 28 |
| 11 | 107 | 116 | MMMLPATLAF | B7 | B*3501 | 10 | DENV3 | 57 | 555 |
| 12 | 108 | 116 | MLIPTAMAF | B7 | B*3501 | 9 | DENV2 | 58 | 422 |
| 13 | 150 | 159 | TLMAMDLGEL | A2 | A*0201 | 10 | DENV2 | 67 | 15 |
| 14 | 164 | 172 | VTYECPLLV | A2 | A*0201 | 9 | DENV4 | 40 | 27 |
| 15 | 245 | 254 | HPGFTILALF | B7 | B*3501 | 10 | DENV3 | 63 | 118 |
| 16 | 248 | 257 | FTIMAAILAY | B7 | B*3501 | 10 | DENV2 | 53 | 4223 |
| 17 | 248 | 257 | FTILALFLAH | B7 | B*3501 | 10 | DENV3 | 32 | 24988 |
| 18 | 249 | 257 | TIMAAILAY | B7 | B*3501 | 9 | DENV2 | 123 | 82 |
| 19 | 274 | 282 | MLVTPSMTM | B7 | B*3501 | 9 | DENV3 | 115 | 3850 |
| 20 | 355 | 363 | CPTQGEATL | B7 | B*3501 | 9 | DENV1 | 143 | 26 |
| 21 | 355 | 363 | CPTQGEAVL | B7 | B*3501 | 9 | DENV3 | 135 | 19 |
| 22 | 363 | 371 | LPEEQDQNY | B7 | B*3501 | 9 | DENV3 | 28 | 1015 |
| 23 | 413 | 421 | YENLKYSVI | B44 | B*4402 | 9 | DENV1 | 37 | 90 |
| 24 | 537 | 545 | QEGAMHSAL | B44 | B*4001 | 9 | DENV4 | 22 | 16 |
| 25 | 537 | 545 | QEGAMHTAL | B44 | B*4001 | 9 | DENV1 | 120 | 5 |
| 26 | 578 | 586 | MSYTMCSGK | A3 | A*1101 | 9 | DENV4 | 48 | 27 |
| 27 | 578 | 587 | MSYSMCTGKF | B7 | B*3501 | 10 | DENV2 | 23 | 10625 |
| 28 | 612 | 621 | SPCKIPFEIM | B7 | B*3501 | 10 | DENV2 | 35 | 7486 |
| 29 | 616 | 625 | IPFEIMDLEK | B7 | B*3501 | 10 | DENV2 | 237 | 6012 |
| 30 | 664 | 673 | EPGQLKLNWF | B7 | B*3501 | 10 | DENV2 | 168 | 42066 |
| 31 | 721 | 729 | FGAIYGAAF | B7 | B*3501 | 9 | DENV2 | 28 | 7667 |
| 32 | 733 | 742 | SWMVRILIGF | A24 | A*2402 | 10 | DENV4 | 90 | 132 |
| 33 | 738 | 746 | IGIGILLTW | B58 | B*5801 | 9 | DENV1 | 23 | 3 |
| 34 | 814 | 823 | SPKRLATAIA | B7 | B*0702 | 10 | DENV3 | 102 | 34 |
| 35 | 845 | 853 | KQIANELNY | B62 | B*1501 | 9 | DENV3 | 22 | 9 |
| 36 | 950 | 959 | VYTQLCDHRL | A24 | A*2402 | 10 | DENV3 | 67 | 6 |
| 37 | 950 | 958 | VYTQLCDHR | A3 | A*3301 | 9 | DENV3 | 28 | 1902 |
| 38 | 968 | 977 | KAVHADMGYW | B58 | B*5801 | 10 | DENV1 | 85 | 1 |
| 39 | 990 | 999 | RASFIEVKTC | B58 | B*5801 | 10 | DENV1 | 138 | 54 |
| 40 | 1023 | 1032 | FAGPVSQHNY | B7 | B*3501 | 10 | DENV2 | 190 | 38 |
| 41 | 1033 | 1041 | RPGYHTQTA | B7 | B*0702 | 9 | DENV2 | 177 | 10 |
| 42 | 1042 | 1051 | GPWHLGKLEL | B7 | B*0702 | 10 | DENV1 | 53 | 18 |
| 43 | 1042 | 1051 | GPWHLGKLEM | B7 | B*3501 | 10 | DENV2 | 25 | 6069 |
| 44 | 1098 | 1107 | RYMGEDGCWY | A24 | A*2402 | 10 | DENV3 | 182 | 829 |
| 45 | 1136 | 1145 | FTMGVLCLAI | A2 | A*0201 | 10 | DENV3 | 33 | 18 |
| 46 | 1176 | 1185 | MSFRDLGRVM | B7 | B*3501 | 10 | DENV2 | 35 | 469 |
| 47 | 1201 | 1209 | TYLALIATF | A24 | A*2402 | 9 | DENV3 | 82 | 7 |
| 48 | 1211 | 1219 | IQPFLALGF | A24 | A*2402 | 9 | DENV3 | 27 | 268 |
| 49 | 1218 | 1227 | GFFLRKLTSR | A3 | A*3301 | 10 | DENV3 | 230 | 59 |
| 50 | 1230 | 1238 | MMATIGIAL | B7 | B*3501 | 9 | DENV2 | 38 | 1117 |
| 51 | 1298 | 1306 | MALSIVSLF | B7 | B*5101 | 9 | DENV1 | 340 | 605 |
| 52 | 1356 | 1364 | MAVGMVSIL | B7 | B*3501 | 9 | DENV2 | 172 | 10 |
| 53 | 1373 | 1382 | IPMTGPLVAG | B7 | B*3501 | 10 | DENV2 | 182 | 129 |
| 54 | 1377 | 1385 | GPLVAGGLL | B7 | B*0702 | 9 | DENV2 | 35 | 67 |
| 55 | 1418 | 1427 | SPILSITISE | B7 | B*3501 | 10 | DENV2 | 158 | 4189 |
| 56 | 1457 | 1466 | FPVSIPITAA | B7 | B*3501 | 10 | DENV2 | 35 | 14 |
| 57 | 1461 | 1469 | IPITAAAWY | B7 | B*3501 | 9 | DENV2 | 70 | 6 |
| 58 | 1519 | 1527 | MEGVFHTMW | B44 | B*4403 | 9 | DENV4 | 68 | 3 |
| 59 | 1519 | 1528 | MEGVFHTMWH | B44 | B*4403 | 10 | DENV4 | 107 | 73 |
| 60 | 1608 | 1616 | KPGTSGSPI | B7 | B*0702 | 9 | DENV1 | 350 | 2 |
| 61 | 1608 | 1617 | KPGTSGSPII | B7 | B*0702 | 10 | DENV3 | 365 | 35 |
| 62 | 1610 | 1619 | GTSGSPIIDK | A3 | A*1101 | 10 | DENV2 | 32 | 12 |
| 63 | 1614 | 1623 | SPIINREGKV | B7 | B*0702 | 10 | DENV3 | 105 | 313 |
| 64 | 1653 | 1661 | NPEIEDDIF | B7 | B*3501 | 9 | DENV2 | 110 | 518 |
| 65 | 1672 | 1681 | HPGAGKTKRY | B7 | B*3501 | 10 | DENV2 | 108 | 680 |
| 66 | 1682 | 1690 | LPAIVREAI | B7 | B*0702 | 9 | DENV1 | 137 | 7 |
| 67 | 1700 | 1709 | APTRVVAAEM | B7 | B*3501 | 10 | DENV2 | 135 | 20 |
| 68 | 1700 | 1709 | APTRVVASEM | B7 | B*0702 | 10 | DENV1 | 153 | 8 |
| 69 | 1700 | 1709 | APTRVVAAEM | B7 | B*0702 | 10 | DENV2 | 113 | 5 |
| 70 | 1707 | 1716 | SEMAEALKGM | B44 | B*4001 | 10 | DENV1 | 120 | 613 |
| 71 | 1716 | 1724 | LPIRYQTPA | B7 | B*0702 | 9 | DENV2 | 180 | 19 |
| 72 | 1716 | 1725 | LPIRYQTPAI | B7 | B*3501 | 10 | DENV2 | 195 | 52 |
| 73 | 1768 | 1777 | DPASIAARGY | B7 | B*3501 | 10 | DENV1 | 183 | 5623 |
| 74 | 1769 | 1778 | PASIAARGYI | B58 | B*5801 | 10 | DENV1 | 140 | 263 |
| 75 | 1795 | 1803 | TPPGSRDPF | B7 | B*3501 | 9 | DENV2 | 210 | 161 |
| 76 | 1803 | 1812 | FPQSNAPIMD | B7 | B*3501 | 10 | DENV2 | 107 | 1 |
| 77 | 1803 | 1811 | FPQSNAPIM | B7 | B*3501 | 9 | DENV2 | 127 | 13693 |
| 78 | 1813 | 1822 | EERDIPERSW | B44 | B*4403 | 10 | DENV1 | 190 | 410 |
| 79 | 1815 | 1824 | REIPERSWNT | B44 | B*4001 | 10 | DENV4 | 93 | 1488 |
| 80 | 1872 | 1881 | YPKTKLTDWD | B7 | B*3501 | 10 | DENV4 | 267 | 1317 |
| 81 | 1899 | 1908 | RVIDPRRCMK | A3 | A*1101 | 10 | DENV2 | 93 | 64 |
| 82 | 1899 | 1908 | RVIDPRRCLK | A3 | A*1101 | 10 | DENV1 | 117 | 58 |
| 83 | 1899 | 1908 | RVIDPRRCMK | A3 | A*3101 | 10 | DENV2 | 115 | 4 |
| 84 | 1899 | 1907 | RVIDPRRCL | B7 | B*0702 | 9 | DENV1 | 117 | 146 |
| 85 | 1899 | 1908 | RVIDPRRCMK | A3 | A*0301 | 10 | DENV2 | 160 | 13 |
| 86 | 1902 | 1910 | DPRRCLKPV | B7 | B*0702 | 9 | DENV1 | 115 | 225 |
| 87 | 1925 | 1933 | MPVTHSSAA | B7 | B*3501 | 9 | DENV2 | 60 | 73 |
| 88 | 1925 | 1934 | MPVTHSSAAQ | B7 | B*3501 | 10 | DENV2 | 25 | 933 |
| 89 | 1942 | 1950 | NPAQEDDQY | B7 | B*3501 | 9 | DENV4 | 118 | 136 |
| 90 | 1949 | 1957 | QYIFTGQPL | A24 | A*2402 | 9 | DENV3 | 78 | 271 |
| 91 | 1978 | 1986 | TPEGIIPSM | B7 | B*0702 | 9 | DENV2 | 108 | 254 |
| 92 | 1978 | 1987 | TPEGIIPSMF | B7 | B*0702 | 10 | DENV2 | 27 | 12953 |
| 93 | 1978 | 1986 | TPEGIIPAL | B7 | B*0702 | 9 | DENV1 | 57 | 1214 |
| 94 | 1978 | 1987 | TPEGIIPALF | B7 | B*0702 | 10 | DENV1 | 38 | 1392 |
| 95 | 1978 | 1986 | TPEGIIPSM | B7 | B*3501 | 9 | DENV2 | 295 | 8 |
| 96 | 1978 | 1987 | TPEGIIPSMF | B7 | B*3501 | 10 | DENV2 | 297 | 386 |
| 97 | 1978 | 1987 | TPEGIIPTLF | B7 | B*3501 | 10 | DENV4 | 90 | 94 |
| 98 | 1978 | 1987 | TPEGIIPALF | B7 | B*3501 | 10 | DENV1 | 20 | 160 |
| 99 | 1999 | 2008 | GEFRLRGEQR | B44 | B*4001 | 10 | DENV4 | 273 | 1407 |
| 100 | 2005 | 2014 | GEARKTFVEL | B44 | B*4001 | 10 | DENV1 | 95 | 7 |
| 101 | 2005 | 2014 | GEARKTFVDL | B44 | B*4001 | 10 | DENV2 | 87 | 5 |
| 102 | 2005 | 2014 | GESRKTFVEL | B44 | B*4001 | 10 | DENV3 | 92 | 4 |
| 103 | 2005 | 2014 | GEQRKTFVEL | B44 | B*4001 | 10 | DENV4 | 37 | 5 |
| 104 | 2013 | 2022 | ELMRRGDLPV | A2 | A*0201 | 10 | DENV1 | 28 | 22 |
| 105 | 2020 | 2029 | LPVWLAYKVA | B7 | B*3501 | 10 | DENV2 | 27 | 5097 |
| 106 | 2026 | 2035 | YKVASAGISY | B7 | B*3501 | 10 | DENV4 | 238 | 70 |
| 107 | 2038 | 2047 | REWCFTGERN | B44 | B*4001 | 10 | DENV4 | 48 | 502 |
| 108 | 2070 | 2078 | RPRWLDART | B7 | B*0702 | 9 | DENV1 | 113 | 2 |
| 109 | 2083 | 2091 | MALKDFKEF | B7 | B*3501 | 9 | DENV4 | 40 | 77 |
| 110 | 2087 | 2095 | EFKEFAAGR | A3 | A*3301 | 9 | DENV1 | 60 | 2 |
| 111 | 2091 | 2100 | FASGRKSITL | B58 | B*5801 | 10 | DENV4 | 72 | 5541 |
| 112 | 2109 | 2118 | LPTFMTQKAR | B7 | B*3501 | 10 | DENV2 | 53 | 176 |
| 113 | 2113 | 2121 | MTQKARNAL | B7 | B*0702 | 9 | DENV2 | 263 | 16 |
| 114 | 2129 | 2137 | TAEAGGRAY | B7 | B*3501 | 9 | DENV2 | 230 | 46 |
| 115 | 2144 | 2153 | LPETLETLLL | B7 | B*3501 | 10 | DENV2 | 512 | 1693 |
| 116 | 2148 | 2156 | LETLMLVAL | B44 | B*4001 | 9 | DENV4 | 112 | 3 |
| 117 | 2148 | 2157 | LETLMLVALL | B44 | B*4001 | 10 | DENV4 | 185 | 127 |
| 118 | 2150 | 2159 | TLMLLALIAV | A2 | A*0201 | 10 | DENV1 | 50 | 8 |
| 119 | 2151 | 2160 | LMLLALIAVL | A2 | A*0201 | 10 | DENV1 | 63 | 95 |
| 120 | 2152 | 2160 | MLLALIAVL | A2 | A*0201 | 9 | DENV1 | 85 | 9 |
| 121 | 2163 | 2172 | GAMLFLISGK | A3 | A*1101 | 10 | DENV3 | 212 | 43 |
| 122 | 2204 | 2213 | SIILEFFLMV | A2 | A*0201 | 10 | DENV1 | 737 | 10 |
| 123 | 2205 | 2213 | IILEFFLMV | A2 | A*0201 | 9 | DENV1 | 232 | 75 |
| 124 | 2205 | 2214 | IILEFFLMVL | A2 | A*0201 | 10 | DENV1 | 152 | 96 |
| 125 | 2210 | 2219 | FLMVLLIPEP | A2 | A*0201 | 10 | DENV1 | 98 | 31 |
| 126 | 2224 | 2233 | TPQDNQLAYV | B7 | B*0702 | 10 | DENV1 | 100 | 331 |
| 127 | 2224 | 2232 | TPQDNQLTY | B7 | B*3501 | 9 | DENV2 | 22 | 11 |
| 128 | 2254 | 2263 | TTKRDLGMSK | A3 | A*1101 | 10 | DENV3 | 75 | 116 |
| 129 | 2266 | 2279 | TETTILDVDL | B44 | B*4001 | 10 | DENV4 | 852 | 11 |
| 130 | 2280 | 2288 | RPASAWTLY | B7 | B*0702 | 9 | DENV1 | 118 | 159 |
| 131 | 2280 | 2289 | RPASAWTLYA | B7 | B*0702 | 10 | DENV1 | 115 | 7 |
| 132 | 2280 | 2288 | HPASAWTLY | B7 | B*3501 | 9 | DENV1 | 38 | 6 |
| 133 | 2281 | 2290 | PASAWTLYAV | B58 | B*5801 | 10 | DENV1 | 90 | 704 |
| 134 | 2290 | 2298 | VATTFVTPM | B7 | B*3501 | 9 | DENV2 | 268 | 205 |
| 135 | 2295 | 2303 | ITPMLRHTI | A24 | A*2402 | 9 | DENV3 | 193 | 138 |
| 136 | 2296 | 2305 | TPMLRHTIEN | B7 | B*0702 | 10 | DENV3 | 90 | 1037 |
| 137 | 2315 | 2323 | IANQATVLM | B7 | B*3501 | 9 | DENV2 | 220 | 16 |
| 138 | 2338 | 2346 | VPLLAIGCY | B7 | B*3501 | 9 | DENV2 | 213 | 168 |
| 139 | 2350 | 2358 | NPLTLTAAV | B7 | B*0702 | 9 | DENV1 | 92 | 32 |
| 140 | 2353 | 2362 | TLTAAVLLLV | A2 | A*0201 | 10 | DENV3 | 43 | 179 |
| 141 | 2356 | 2365 | AAVLLLVTHY | B58 | B*5801 | 10 | DENV3 | 102 | 4148 |
| 142 | 2358 | 2367 | VLLLVTHYAI | A2 | A*0201 | 10 | DENV3 | 260 | 219 |
| 143 | 2403 | 2411 | DPIPYDPKF | B7 | B*3501 | 9 | DENV2 | 77 | 166 |
| 144 | 2419 | 2428 | MLLILCVTQV | A2 | A*0201 | 10 | DENV2 | 103 | 4 |
| 145 | 2444 | 2452 | ATGPLTTLW | B58 | B*5801 | 9 | DENV1 | 350 | 7 |
| 146 | 2444 | 2452 | ATGPISTLW | B58 | B*5801 | 9 | DENV2 | 163 | 1 |
| 147 | 2444 | 2452 | ATGPITTLW | B58 | B*5801 | 9 | DENV3 | 110 | 5 |
| 148 | 2444 | 2452 | ATGPILTLW | B58 | B*5801 | 9 | DENV4 | 27 | 13 |
| 149 | 2444 | 2452 | ATGPVLTLW | B58 | B*5801 | 9 | DENV4 | 185 | 0 |
| 150 | 2451 | 2459 | LWEGSPGKF | A24 | A*2402 | 9 | DENV1 | 57 | 6165 |
| 151 | 2455 | 2464 | SPGKFWNTTI | B7 | B*0702 | 10 | DENV1 | 105 | 6 |
| 152 | 2464 | 2472 | IAVSMANIF | B7 | B*3501 | 9 | DENV1 | 118 | 143 |
| 153 | 2464 | 2472 | IAVSMANIF | B58 | B*5801 | 9 | DENV2 | 108 | 52 |
| 154 | 2464 | 2472 | IAVSTANIF | B58 | B*5801 | 9 | DENV4 | 135 | 196 |
| 155 | 2468 | 2476 | MANIFRGSY | B7 | B*3501 | 9 | DENV1 | 5982 | 553 |
| 156 | 2476 | 2484 | YLAGAGLAF | B7 | B*0702 | 9 | DENV1 | 72 | 98 |
| 157 | 2553 | 2562 | GSSKIRWIVE | B58 | B*5801 | 10 | DENV4 | 45 | 219 |
| 158 | 2602 | 2611 | GPGHEEPIPM | B7 | B*3501 | 10 | DENV1 | 53 | 1150 |
| 159 | 2609 | 2618 | IPMSTYGWNL | B7 | B*0702 | 10 | DENV2 | 203 | 59 |
| 160 | 2609 | 2618 | IPMATYGWNL | B7 | B*0702 | 10 | DENV1 | 450 | 20 |
| 161 | 2609 | 2618 | IPMSTYGWNL | B7 | B*3501 | 10 | DENV2 | 33 | 393 |
| 162 | 2611 | 2620 | MSTYGWNIVK | A3 | A*1101 | 10 | DENV3 | 30 | 146 |
| 163 | 2612 | 2620 | STYGWNIVK | A3 | A*1101 | 9 | DENV3 | 273 | 21 |
| 164 | 2622 | 2631 | QSGVDVFFTP | B58 | B*5801 | 10 | DENV2 | 387 | 2662 |
| 165 | 2658 | 2666 | RVLKMVEPW | B58 | B*5801 | 9 | DENV1 | 643 | 1 |
| 166 | 2676 | 2685 | KVLNPYMPSV | A2 | A*0201 | 10 | DENV2 | 48 | 8 |
| 167 | 2677 | 2685 | VLNPYMPSV | A2 | A*0201 | 9 | DENV2 | 987 | 1 |
| 168 | 2682 | 2691 | MPSVIEKMET | B7 | B*3501 | 10 | DENV2 | 1010 | 375 |
| 169 | 2724 | 2733 | VSSVNMVSRL | B58 | B*5801 | 10 | DENV3 | 820 | 95 |
| 170 | 2729 | 2737 | MVSRLLLNR | A3 | A*1101 | 9 | DENV3 | 992 | 50 |
| 171 | 2738 | 2747 | FTMRHKKATY | B7 | B*3501 | 10 | DENV2 | 103 | 7441 |
| 172 | 2787 | 2795 | WHYDQDHPY | B7 | B*3501 | 9 | DENV2 | 20 | 7598 |
| 173 | 2791 | 2800 | QENPYRTWAY | B44 | B*4001 | 10 | DENV4 | 992 | 1601 |
| 174 | 2798 | 2806 | WAYHGSYET | B7 | B*3501 | 9 | DENV2 | 265 | 873 |
| 175 | 2798 | 2806 | WAYHGSYEV | B7 | B*5101 | 9 | DENV1 | 97 | 11 |
| 176 | 2840 | 2848 | DTTPFGQQR | A3 | A*6801 | 9 | DENV1 | 40 | 91 |
| 177 | 2842 | 2850 | TPFGQQRVF | B7 | B*3501 | 9 | DENV1 | 48 | 47 |
| 178 | 2860 | 2869 | EPKEGTKKLM | B7 | B*3501 | 10 | DENV2 | 382 | 54438 |
| 179 | 2869 | 2877 | MEITAEWLW | B58 | B*5801 | 9 | DENV3 | 27 | 5 |
| 180 | 2885 | 2894 | KPRICTREEF | B7 | B*0702 | 10 | DENV1 | 133 | 72 |
| 181 | 2885 | 2894 | TPRMCTREEF | B7 | B*0702 | 10 | DENV2 | 60 | 13 |
| 182 | 2885 | 2894 | KPRLCTREEF | B7 | B*0702 | 10 | DENV3 | 48 | 13 |
| 183 | 2885 | 2894 | NPRLCTREEF | B7 | B*0702 | 10 | DENV4 | 25 | 45 |
| 184 | 2885 | 2894 | RPRLCTREEF | B7 | B*0702 | 10 | DENV3 | 102 | 7 |
| 185 | 2885 | 2894 | TPRMCTREEF | B7 | B*3501 | 10 | DENV2 | 38 | 2576 |
| 186 | 2918 | 2926 | RAAVEDEEF | B58 | B*5801 | 9 | DENV3 | 87 | 866 |
| 187 | 2919 | 2928 | EAVEDSRFWE | B58 | B*5801 | 10 | DENV2 | 140 | 1714 |
| 188 | 2964 | 2973 | KGSRAIWYMW | B58 | B*5801 | 10 | DENV1 | 335 | 2 |
| 189 | 2977 | 2986 | RYLEFEALGF | A24 | A*2402 | 10 | DENV3 | 130 | 38 |
| 190 | 2977 | 2986 | RFLEFEALGF | A24 | A*2402 | 10 | DENV1 | 37 | 14 |
| 191 | 2993 | 3002 | FSRENSLSGV | B7 | B*5101 | 10 | DENV1 | 103 | 7587 |
| 192 | 3004 | 3012 | GEGLHKLGY | B44 | B*4403 | 9 | DENV1 | 248 | 281 |
| 193 | 3057 | 3065 | RQLANAIFK | A3 | A*1101 | 9 | DENV3 | 277 | 89 |
| 194 | 3079 | 3088 | TPRGTVMDII | B7 | B*0702 | 10 | DENV2 | 505 | 6 |
| 195 | 3079 | 3088 | TPKGAVMDII | B7 | B*0702 | 10 | DENV4 | 422 | 127 |
| 196 | 3116 | 3124 | RQMEGEGIF | B62 | B*1501 | 9 | DENV2 | 583 | 6 |
| 197 | 3116 | 3124 | RQMEGEGVL | B62 | B*1501 | 9 | DENV3 | 382 | 19 |
| 198 | 3182 | 3190 | KVRKDIQQW | B58 | B*5701 | 9 | DENV2 | 115 | 15 |
| 199 | 3254 | 3262 | YAQMWSLMY | B62 | B*1501 | 9 | DENV2 | 27 | 6 |
| 200 | 3254 | 3263 | YAQMWSLMYF | B7 | B*3501 | 10 | DENV2 | 625 | 177 |
| 201 | 3275 | 3283 | ICSAVPVHW | B58 | B*5801 | 9 | DENV3 | 305 | 6 |
| 202 | 3291 | 3299 | WSIHAHHQW | B58 | B*5801 | 9 | DENV1 | 45 | 1 |
| 203 | 3317 | 3326 | NPNMIDKTPV | B7 | B*0702 | 10 | DENV4 | 207 | 403 |
| 204 | 3317 | 3326 | NPWMEDKTPV | B7 | B*0702 | 10 | DENV2 | 137 | 56 |
| 205 | 3332 | 3341 | VPYLGKREDQ | B7 | B*0702 | 10 | DENV1 | 425 | 1251 |
| 206 | 3338 | 3346 | REDLWCGSL | B44 | B*4001 | 9 | DENV4 | 503 | 2 |
| 207 | 3338 | 3346 | REDQWCGSL | B44 | B*4001 | 9 | DENV1 | 150 | 2 |
| 208 | 3379 | 3388 | MPSMKRFRRE | B7 | B*3501 | 10 | DENV2 | 208 | 30905 |
| 209 | 3387 | 3395 | APFESEGVL | B7 | B*0702 | 9 | DENV4 | 77 | 38 |

1. A method of eliciting, stimulating, inducing, promoting, increasing, or enhancing an anti-Dengue virus T cell response in a subject without eliciting or sensitizing the subject to severe Dengue virus disease upon a secondary or subsequent Dengue virus infection, comprising administering to the subject an amount of a Dengue virus protein or subsequence thereof sufficient to elicit, stimulate, induce, promote, increase or enhance an anti-Dengue virus T cell response in the subject.

2.-3. (canceled)

4. The method of claim 1, wherein the Dengue virus protein comprises or consists of a Dengue virus non-structural protein.

5. (canceled)

6. The method of claim 1, wherein the Dengue virus protein comprises or consists of a Dengue virus structural protein.

7. (canceled)

8. The method of claim 1, wherein the method elicits, stimulates, induces, promotes, increases, or enhances an anti-Dengue virus CD8+ T cell response.

9. The method of claim 8, wherein the anti-Dengue virus CD8+ T cell response is directed and/or protective against a plurality of different Dengue virus serotypes.

10. The method of claim 8, wherein the anti-Dengue virus CD8+ T cell response is directed and/or protective against at least two Dengue virus serotypes selected from DENV1, DENV2, DENV3 and DENV4.

11. The method of claim 1, wherein the protein administered consists of a single Dengue virus serotype.

12. (canceled)

13. The method of claim 1, wherein the protein administered comprises or consists of one or more Dengue virus serotype 1, 2, 3 or 4 proteins.

14.-28. (canceled)

29. The method of claim 1, wherein the severe Dengue virus disease comprises antibody-dependent enhancement of infection.

30. The method of claim 1, wherein the subject has not previously been infected with Dengue virus.

31. The method of claim 1, wherein the subject, prior to administration of the Dengue virus protein, produces antibodies against one or more Dengue virus serotypes.

32. The method of claim 1, wherein the subject has previously been infected with Dengue virus.

33. The method of claim 1, comprising reducing Dengue virus titer, increasing or stimulating Dengue virus clearance, reducing or inhibiting Dengue virus proliferation, reducing or inhibiting increases in Dengue virus titer or Dengue virus proliferation, reducing the amount of a Dengue virus protein or the amount of a Dengue virus nucleic acids, or reducing or inhibiting synthesis of a Dengue virus protein or a Dengue virus nucleic acid.

34. The method of claim 1, comprising preventing, reducing, improving or inhibiting one or more adverse physiological conditions, disorders, illnesses, diseases, symptoms or complications caused by or associated with Dengue virus infection or pathology.

35. The method of claim 1, comprising reducing or inhibiting susceptibility to Dengue virus infection or pathology.

36. The method of claim 1, wherein the Dengue virus protein or subsequence thereof is administered prior to exposure to or infection of the subject with the Dengue virus.

37. The method of claim 1, wherein the Dengue virus protein or subsequence thereof is administered substantially contemporaneously with or following exposure to or infection of the subject with the Dengue virus.

38.-41. (canceled)

42. A composition for use in eliciting, stimulating, inducing, promoting, increasing, or enhancing an anti-Dengue virus T cell response in a subject without sensitizing the subject to severe dengue disease upon a secondary or subsequent Dengue virus infection, the composition comprising an amount of a Dengue virus protein or subsequence thereof sufficient to elicit, stimulate, induce, promote, increase or enhance an anti-Dengue virus T cell response in the subject.

43. A composition for use in vaccinating or providing a subject with protection against a Dengue virus infection without sensitizing the subject to severe dengue disease upon a secondary or subsequent Dengue virus infection, the composition comprising an amount of a Dengue virus protein or subsequence thereof sufficient to vaccinate or provide the subject with protection against the Dengue virus infection.

44.-80. (canceled)


SEQUENCE LISTING (763 sequences)

SEQ ID NO: 1 - protein, 9 aa, Dengue virus
GPMKLVMAF

SEQ ID NO: 2 - protein, 10 aa, Dengue virus
GPMKLVMAFI

SEQ ID NO: 3 - protein, 9 aa, Dengue virus
MAFIAFLRF

SEQ ID NO: 4 - protein, 9 aa, Dengue virus
KSGAIKVLK

SEQ ID NO: 5 - protein, 10 aa, Dengue virus
ITLLCLIPTV

SEQ ID NO: 6 - protein, 10 aa, Dengue virus
CLMMMLPATL

SEQ ID NO: 7 - protein, 9 aa, Dengue virus
TLLCLIPTV

SEQ ID NO: 8 - protein, 9 aa, Dengue virus
LMMMLPATL

SEQ ID NO: 9 - protein, 10 aa, Dengue virus
LMMMLPATLA

SEQ ID NO: 10 - protein, 9 aa, Dengue virus
MMMLPATLA

SEQ ID NO: 11 - protein, 10 aa, Dengue virus
MMMLPATLAF

SEQ ID NO: 12 - protein, 9 aa, Dengue virus
MLIPTAMAF

SEQ ID NO: 13 - protein, 10 aa, Dengue virus
TLMAMDLGEL

SEQ ID NO: 14 - protein, 9 aa, Dengue virus
VTYECPLLV

SEQ ID NO: 15 - protein, 10 aa, Dengue virus
HPGFTILALF

SEQ ID NO: 16 - protein, 10 aa, Dengue virus
FTIMAAILAY

SEQ ID NO: 17 - protein, 10 aa, Dengue virus
FTILALFLAH

SEQ ID NO: 18 - protein, 9 aa, Dengue virus
TIMAAILAY

SEQ ID NO: 19 - protein, 9 aa, Dengue virus
MLVTPSMTM

SEQ ID NO: 20 - protein, 9 aa, Dengue virus
CPTQGEATL

SEQ ID NO: 21 - protein, 9 aa, Dengue virus
CPTQGEAVL

SEQ ID NO: 22 - protein, 9 aa, Dengue virus
LPEEQDQNY

SEQ ID NO: 23 - protein, 9 aa, Dengue virus
YENLKYSVI

SEQ ID NO: 24 - protein, 9 aa, Dengue virus
QEGAMHSAL

SEQ ID NO: 25 - protein, 9 aa, Dengue virus
QEGAMHTAL

SEQ ID NO: 26 - protein, 9 aa, Dengue virus
MSYTMCSGK

SEQ ID NO: 27 - protein, 10 aa, Dengue virus
MSYSMCTGKF

SEQ ID NO: 28 - protein, 10 aa, Dengue virus
SPCKIPFEIM

SEQ ID NO: 29 - protein, 10 aa, Dengue virus
IPFEIMDLEK

SEQ ID NO: 30 - protein, 10 aa, Dengue virus
EPGQLKLNWF

SEQ ID NO: 31 - protein, 9 aa, Dengue virus
FGAIYGAAF

SEQ ID NO: 32 - protein, 10 aa, Dengue virus
SWMVRILIGF

SEQ ID NO: 33 - protein, 9 aa, Dengue virus
IGIGILLTW

SEQ ID NO: 34 - protein, 10 aa, Dengue virus
SPKRLATAIA

SEQ ID NO: 35 - protein, 9 aa, Dengue virus
KQIANELNY

SEQ ID NO: 36 - protein, 10 aa, Dengue virus
VYTQLCDHRL

SEQ ID NO: 37 - protein, 9 aa, Dengue virus
VYTQLCDHR

SEQ ID NO: 38 - protein, 10 aa, Dengue virus
KAVHADMGYW

SEQ ID NO: 39 - protein, 10 aa, Dengue virus
RASFIEVKTC

SEQ ID NO: 40 - protein, 10 aa, Dengue virus
FAGPVSQHNY

SEQ ID NO: 41 - protein, 9 aa, Dengue virus
RPGYHTQTA

SEQ ID NO: 42 - protein, 10 aa, Dengue virus
GPWHLGKLEL

SEQ ID NO: 43 - protein, 10 aa, Dengue virus
GPWHLGKLEM

SEQ ID NO: 44 - protein, 10 aa, Dengue virus
RYMGEDGCWY

SEQ ID NO: 45 - protein, 10 aa, Dengue virus
FTMGVLCLAI

SEQ ID NO: 46 - protein, 10 aa, Dengue virus
MSFRDLGRVM

SEQ ID NO: 47 - protein, 9 aa, Dengue virus
TYLALIATF

SEQ ID NO: 48 - protein, 9 aa, Dengue virus
IQPFLALGF

SEQ ID NO: 49 - protein, 10 aa, Dengue virus
GFFLRKLTSR

SEQ ID NO: 50 - protein, 9 aa, Dengue virus
MMATIGIAL

SEQ ID NO: 51 - protein, 9 aa, Dengue virus
MALSIVSLF

SEQ ID NO: 52 - protein, 9 aa, Dengue virus
MAVGMVSIL

SEQ ID NO: 53 - protein, 10 aa, Dengue virus
IPMTGPLVAG

SEQ ID NO: 54 - protein, 9 aa, Dengue virus
GPLVAGGLL

SEQ ID NO: 55 - protein, 10 aa, Dengue virus
SPILSITISE

SEQ ID NO: 56 - protein, 10 aa, Dengue virus
FPVSIPITAA

SEQ ID NO: 57 - protein, 9 aa, Dengue virus
IPITAAAWY

SEQ ID NO: 58 - protein, 9 aa, Dengue virus
MEGVFHTMW

SEQ ID NO: 59 - protein, 10 aa, Dengue virus
MEGVFHTMWH

SEQ ID NO: 60 - protein, 9 aa, Dengue virus
KPGTSGSPI

SEQ ID NO: 61 - protein, 10 aa, Dengue virus
KPGTSGSPII

SEQ ID NO: 62 - protein, 10 aa, Dengue virus
GTSGSPIIDK

SEQ ID NO: 63 - protein, 10 aa, Dengue virus
SPIINREGKV

SEQ ID NO: 64 - protein, 9 aa, Dengue virus
NPEIEDDIF

SEQ ID NO: 65 - protein, 10 aa, Dengue virus
HPGAGKTKRY

SEQ ID NO: 66 - protein, 9 aa, Dengue virus
LPAIVREAI

SEQ ID NO: 67 - protein, 10 aa, Dengue virus
APTRVVAAEM

SEQ ID NO: 68 - protein, 10 aa, Dengue virus
APTRVVASEM

SEQ ID NO: 69 - protein, 10 aa, Dengue virus
APTRVVAAEM

SEQ ID NO: 70 - protein, 10 aa, Dengue virus
SEMAEALKGM

SEQ ID NO: 71 - protein, 9 aa, Dengue virus
LPIRYQTPA

SEQ ID NO: 72 - protein, 10 aa, Dengue virus
LPIRYQTPAI

SEQ ID NO: 73 - protein, 10 aa, Dengue virus
DPASIAARGY

SEQ ID NO: 74 - protein, 10 aa, Dengue virus
PASIAARGYI

SEQ ID NO: 75 - protein, 9 aa, Dengue virus
TPPGSRDPF

SEQ ID NO: 76 - protein, 10 aa, Dengue virus
FPQSNAPIMD

SEQ ID NO: 77 - protein, 9 aa, Dengue virus
FPQSNAPIM

SEQ ID NO: 78 - protein, 10 aa, Dengue virus
EERDIPERSW

SEQ ID NO: 79 - protein, 10 aa, Dengue virus
REIPERSWNT

SEQ ID NO: 80 - protein, 10 aa, Dengue virus
YPKTKLTDWD

SEQ ID NO: 81 - protein, 10 aa, Dengue virus
RVIDPRRCMK

SEQ ID NO: 82 - protein, 10 aa, Dengue virus
RVIDPRRCLK

SEQ ID NO: 83 - protein, 10 aa, Dengue virus
RVIDPRRCMK

SEQ ID NO: 84 - protein, 9 aa, Dengue virus
RVIDPRRCL

SEQ ID NO: 85 - protein, 10 aa, Dengue virus
RVIDPRRCMK

SEQ ID NO: 86 - protein, 9 aa, Dengue virus
DPRRCLKPV

SEQ ID NO: 87 - protein, 9 aa, Dengue virus
MPVTHSSAA

SEQ ID NO: 88 - protein, 10 aa, Dengue virus
MPVTHSSAAQ

SEQ ID NO: 89 - protein, 9 aa, Dengue virus
NPAQEDDQY

SEQ ID NO: 90 - protein, 9 aa, Dengue virus
QYIFTGQPL

SEQ ID NO: 91 - protein, 9 aa, Dengue virus
TPEGIIPSM

SEQ ID NO: 92 - protein, 10 aa, Dengue virus
TPEGIIPSMF

SEQ ID NO: 93 - protein, 9 aa, Dengue virus
TPEGIIPAL

SEQ ID NO: 94 - protein, 10 aa, Dengue virus
TPEGIIPALF

SEQ ID NO: 95 - protein, 9 aa, Dengue virus
TPEGIIPSM

SEQ ID NO: 96 - protein, 10 aa, Dengue virus
TPEGIIPSMF

SEQ ID NO: 97 - protein, 10 aa, Dengue virus
TPEGIIPTLF

SEQ ID NO: 98 - protein, 10 aa, Dengue virus
TPEGIIPALF

SEQ ID NO: 99 - protein, 10 aa, Dengue virus
GEFRLRGEQR

SEQ ID NO: 100 - protein, 10 aa, Dengue virus
GEARKTFVEL

SEQ ID NO: 101 - protein, 10 aa, Dengue virus
GEARKTFVDL

SEQ ID NO: 102 - protein, 10 aa, Dengue virus
GESRKTFVEL

SEQ ID NO: 103 - protein, 10 aa, Dengue virus
GEQRKTFVEL

SEQ ID NO: 104 - protein, 10 aa, Dengue virus
ELMRRGDLPV

SEQ ID NO: 105 - protein, 10 aa, Dengue virus
LPVWLAYKVA

SEQ ID NO: 106 - protein, 10 aa, Dengue virus
YKVASAGISY

SEQ ID NO: 107 - protein, 10 aa, Dengue virus
REWCFTGERN

SEQ ID NO: 108 - protein, 9 aa, Dengue virus
RPRWLDART

SEQ ID NO: 109 - protein, 9 aa, Dengue virus
MALKDFKEF

SEQ ID NO: 110 - protein, 9 aa, Dengue virus
EFKEFAAGR

SEQ ID NO: 111 - protein, 10 aa, Dengue virus
FASGRKSITL

SEQ ID NO: 112 - protein, 10 aa, Dengue virus
LPTFMTQKAR

SEQ ID NO: 113 - protein, 9 aa, Dengue virus
MTQKARNAL

SEQ ID NO: 114 - protein, 9 aa, Dengue virus
TAEAGGRAY

SEQ ID NO: 115 - protein, 10 aa, Dengue virus
LPETLETLLL

SEQ ID NO: 116 - protein, 9 aa, Dengue virus
LETLMLVAL

SEQ ID NO: 117 - protein, 10 aa, Dengue virus
LETLMLVALL

SEQ ID NO: 118 - protein, 10 aa, Dengue virus
TLMLLALIAV

SEQ ID NO: 119 - protein, 10 aa, Dengue virus
LMLLALIAVL

SEQ ID NO: 120 - protein, 9 aa, Dengue virus
MLLALIAVL

SEQ ID NO: 121 - protein, 10 aa, Dengue virus
GAMLFLISGK

SEQ ID NO: 122 - protein, 10 aa, Dengue virus
SIILEFFLMV

SEQ ID NO: 123 - protein, 9 aa, Dengue virus
IILEFFLMV

SEQ ID NO: 124 - protein, 10 aa, Dengue virus
IILEFFLMVL

SEQ ID NO: 125 - protein, 10 aa, Dengue virus
FLMVLLIPEP

SEQ ID NO: 126 - protein, 10 aa, Dengue virus
TPQDNQLAYV

SEQ ID NO: 127 - protein, 9 aa, Dengue virus
TPQDNQLTY

SEQ ID NO: 128 - protein, 10 aa, Dengue virus
TTKRDLGMSK

SEQ ID NO: 129 - protein, 10 aa, Dengue virus
TETTILDVDL

SEQ ID NO: 130 - protein, 9 aa, Dengue virus
RPASAWTLY

SEQ ID NO: 131 - protein, 10 aa, Dengue virus
RPASAWTLYA

SEQ ID NO: 132 - protein, 9 aa, Dengue virus
HPASAWTLY

SEQ ID NO: 133 - protein, 10 aa, Dengue virus
PASAWTLYAV

SEQ ID NO: 134 - protein, 9 aa, Dengue virus
VATTFVTPM

SEQ ID NO: 135 - protein, 9 aa, Dengue virus
ITPMLRHTI

SEQ ID NO: 136 - protein, 10 aa, Dengue virus
TPMLRHTIEN

SEQ ID NO: 137 - protein, 9 aa, Dengue virus
IANQATVLM

SEQ ID NO: 138 - protein, 9 aa, Dengue virus
VPLLAIGCY

SEQ ID NO: 139 - protein, 9 aa, Dengue virus
NPLTLTAAV

SEQ ID NO: 140 - protein, 10 aa, Dengue virus
TLTAAVLLLV

SEQ ID NO: 141 - protein, 10 aa, Dengue virus
AAVLLLVTHY

SEQ ID NO: 142 - protein, 10 aa, Dengue virus
VLLLVTHYAI

SEQ ID NO: 143 - protein, 9 aa, Dengue virus
DPIPYDPKF

SEQ ID NO: 144 - protein, 10 aa, Dengue virus
MLLILCVTQV

SEQ ID NO: 145 - protein, 9 aa, Dengue virus
ATGPLTTLW

SEQ ID NO: 146 - protein, 9 aa, Dengue virus
ATGPISTLW

SEQ ID NO: 147 - protein, 9 aa, Dengue virus
ATGPITTLW

SEQ ID NO: 148 - protein, 9 aa, Dengue virus
ATGPILTLW

SEQ ID NO: 149 - protein, 9 aa, Dengue virus
ATGPVLTLW

SEQ ID NO: 150 - protein, 9 aa, Dengue virus
LWEGSPGKF

SEQ ID NO: 151 - protein, 10 aa, Dengue virus
SPGKFWNTTI

SEQ ID NO: 152 - protein, 9 aa, Dengue virus
IAVSMANIF

SEQ ID NO: 153 - protein, 9 aa, Dengue virus
IAVSMANIF

SEQ ID NO: 154 - protein, 9 aa, Dengue virus
IAVSTANIF

SEQ ID NO: 155 - protein, 9 aa, Dengue virus
MANIFRGSY

SEQ ID NO: 156 - protein, 9 aa, Dengue virus
YLAGAGLAF

SEQ ID NO: 157 - protein, 10 aa, Dengue virus
GSSKIRWIVE

SEQ ID NO: 158 - protein, 10 aa, Dengue virus
GPGHEEPIPM

SEQ ID NO: 159 - protein, 10 aa, Dengue virus
IPMSTYGWNL

SEQ ID NO: 160 - protein, 10 aa, Dengue virus
IPMATYGWNL

SEQ ID NO: 161 - protein, 10 aa, Dengue virus
IPMSTYGWNL

SEQ ID NO: 162 - protein, 10 aa, Dengue virus
MSTYGWNIVK

SEQ ID NO: 163 - protein, 9 aa, Dengue virus
STYGWNIVK

SEQ ID NO: 164 - protein, 10 aa, Dengue virus
QSGVDVFFTP

SEQ ID NO: 165 - protein, 9 aa, Dengue virus
RVLKMVEPW

SEQ ID NO: 166 - protein, 10 aa, Dengue virus
KVLNPYMPSV

SEQ ID NO: 167 - protein, 9 aa, Dengue virus
VLNPYMPSV

SEQ ID NO: 168 - protein, 10 aa, Dengue virus
MPSVIEKMET

SEQ ID NO: 169 - protein, 10 aa, Dengue virus
VSSVNMVSRL

SEQ ID NO: 170 - protein, 9 aa, Dengue virus
MVSRLLLNR

SEQ ID NO: 171 - protein, 10 aa, Dengue virus
FTMRHKKATY

SEQ ID NO: 172 - protein, 9 aa, Dengue virus
WHYDQDHPY

SEQ ID NO: 173 - protein, 10 aa, Dengue virus
QENPYRTWAY

SEQ ID NO: 174 - protein, 9 aa, Dengue virus
WAYHGSYET

SEQ ID NO: 175 - protein, 9 aa, Dengue virus
WAYHGSYEV

SEQ ID NO: 176 - protein, 9 aa, Dengue virus
DTTPFGQQR

SEQ ID NO: 177 - protein, 9 aa, Dengue virus
TPFGQQRVF

SEQ ID NO: 178 - protein, 10 aa, Dengue virus
EPKEGTKKLM

SEQ ID NO: 179 - protein, 9 aa, Dengue virus
MEITAEWLW

SEQ ID NO: 180 - protein, 10 aa, Dengue virus
KPRICTREEF

SEQ ID NO: 181 - protein, 10 aa, Dengue virus
TPRMCTREEF

SEQ ID NO: 182 - protein, 10 aa, Dengue virus
KPRLCTREEF

SEQ ID NO: 183 - protein, 10 aa, Dengue virus
NPRLCTREEF

SEQ ID NO: 184 - protein, 10 aa, Dengue virus
RPRLCTREEF

SEQ ID NO: 185 - protein, 10 aa, Dengue virus
TPRMCTREEF

SEQ ID NO: 186 - protein, 9 aa, Dengue virus
RAAVEDEEF

SEQ ID NO: 187 - protein, 10 aa, Dengue virus
EAVEDSRFWE

SEQ ID NO: 188 - protein, 10 aa, Dengue virus
KGSRAIWYMW

SEQ ID NO: 189 - protein, 10 aa, Dengue virus
RYLEFEALGF

SEQ ID NO: 190 - protein, 10 aa, Dengue virus
RFLEFEALGF

SEQ ID NO: 191 - protein, 10 aa, Dengue virus
FSRENSLSGV

SEQ ID NO: 192 - protein, 9 aa, Dengue virus
GEGLHKLGY

SEQ ID NO: 193 - protein, 9 aa, Dengue virus
RQLANAIFK

SEQ ID NO: 194 - protein, 10 aa, Dengue virus
TPRGTVMDII

SEQ ID NO: 195 - protein, 10 aa, Dengue virus
TPKGAVMDII

SEQ ID NO: 196 - protein, 9 aa, Dengue virus
RQMEGEGIF

SEQ ID NO: 197 - protein, 9 aa, Dengue virus
RQMEGEGVL

SEQ ID NO: 198 - protein, 9 aa, Dengue virus
KVRKDIQQW

SEQ ID NO: 199 - protein, 9 aa, Dengue virus
YAQMWSLMY

SEQ ID NO: 200 - protein, 10 aa, Dengue virus
YAQMWSLMYF

SEQ ID NO: 201 - protein, 9 aa, Dengue virus
ICSAVPVHW

SEQ ID NO: 202 - protein, 9 aa, Dengue virus
WSIHAHHQW

SEQ ID NO: 203 - protein, 10 aa, Dengue virus
NPNMIDKTPV

SEQ ID NO: 204 - protein, 10 aa, Dengue virus
NPWMEDKTPV

SEQ ID NO: 205 - protein, 10 aa, Dengue virus
VPYLGKREDQ

SEQ ID NO: 206 - protein, 9 aa, Dengue virus
REDLWCGSL

SEQ ID NO: 207 - protein, 9 aa, Dengue virus
REDQWCGSL

SEQ ID NO: 208 - protein, 10 aa, Dengue virus
MPSMKRFRRE

SEQ ID NO: 209 - protein, 9 aa, Dengue virus
APFESEGVL

SEQ ID NO: 210 - protein, 114 aa, Dengue virus
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGMIIMLIPTVMA

SEQ ID NO: 211 - protein, 166 aa, Dengue virus
FHLTTRNGEPHMIVSRQEKGKSLLFKTGDGVNMCTLMAMDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 212 - protein, 495 aa, Dengue virus
MRCIGISNRDFVEGVSGGSWVDIVLEHGSCVTTMAKNKPTLDFELIKTEAKQSATLRKYCIEAKLTNTTTESRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMLETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 213 - protein, 353 aa, Dengue virus
ADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKSHTLWSNEVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTA

SEQ ID NO: 214 - protein, 218 aa, Dengue virus
GHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRKMEKYQLAVTIMAILCVPNAVILQNAWKVSCTILAVVSVSPLFLTSSQQKADWIPLALTIKGLNPTAIFLTTLSRTNKKR

SEQ ID NO: 215 - protein, 130 aa, Dengue virus
SWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSLPITAAAWYLWEVKKQR

SEQ ID NO: 216 - protein, 618 aa, Dengue virus
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDARIYSDPLALKEFKEFAAGRK

SEQ ID NO: 217 - protein, 150 aa, Dengue virus
SLTLSLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMA

SEQ ID NO: 218 - protein, 248 aa, Dengue virus
NEMGFLEKTKKDLGLGSITTQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRR

SEQ ID NO: 219 - protein, 900 aa, Dengue virus
GTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTVTEEIAVQNWLARVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTAEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 220 - protein, 15 aa, Dengue virus
GLFPVSLPITAAAWY

SEQ ID NO: 221 - protein, 15 aa, Dengue virus
GKTKRYLPAIVREAI

SEQ ID NO: 222 - protein, 15 aa, Dengue virus
GLPIRYQTPAIRAEH

SEQ ID NO: 223 - protein, 15 aa, Dengue virus
IGCYSQVNPITLTAA

SEQ ID NO: 224 - protein, 9 aa, Dengue virus
ITEAELTGY

SEQ ID NO: 225 - protein, 10 aa, Dengue virus
RSCTLPPLRY

SEQ ID NO: 226 - protein, 9 aa, Dengue virus
MTDDIGMGV

SEQ ID NO: 227 - protein, 9 aa, Dengue virus
LTDALALGM

SEQ ID NO: 228 - protein, 9 aa, Dengue virus
VIDLDPIPY

SEQ ID NO: 229 - protein, 9 aa, Dengue virus
YTDYMPSMK

SEQ ID NO: 230 - protein, 9 aa, Dengue virus
RLITVNPIV

SEQ ID NO: 231 - protein, 9 aa, Dengue virus
IMAVGMVSI

SEQ ID NO: 232 - protein, 9 aa, Dengue virus
GLLTVCYVL

SEQ ID NO: 233 - protein, 10 aa, Dengue virus
LLVISGLFPV

SEQ ID NO: 234 - protein, 9 aa, Dengue virus
AAAWYLWEV

SEQ ID NO: 235 - protein, 9 aa, Dengue virus
YLPAIVREA

SEQ ID NO: 236 - protein, 10 aa, Dengue virus
DLMRRGDLPV

SEQ ID NO: 237 - protein, 9 aa, Dengue virus
ALSELPETL

SEQ ID NO: 238 - protein, 9 aa, Dengue virus
IILEFFLIV

SEQ ID NO: 239 - protein, 9 aa, Dengue virus
KLAEAIFKL

SEQ ID NO: 240 - protein, 9 aa, Dengue virus
SQIGAGVYK

SEQ ID NO: 241 - protein, 10 aa, Dengue virus
GTSGSPIIDK

SEQ ID NO: 242 - protein, 9 aa, Dengue virus
KTFDSEYVK

SEQ ID NO: 243 - protein, 10 aa, Dengue virus
RIYSDPLALK

SEQ ID NO: 244 - protein, 9 aa, Dengue virus
ATVLMGLGK

SEQ ID NO: 245 - protein, 9 aa, Dengue virus
STYGWNLVR

SEQ ID NO: 246 - protein, 9 aa, Dengue virus
TVMDIISRR

SEQ ID NO: 247 - protein, 10 aa, Dengue virus
RQMEGEGVFK

SEQ ID NO: 248 - protein, 9 aa, Dengue virus
RTTWSIHAK

SEQ ID NO: 249 - protein, 10 aa, Dengue virus
RPTFAAGLLL

SEQ ID NO: 250 - protein, 9 aa, Dengue virus
LPAIVREAI

SEQ ID NO: 251 - protein, 10 aa, Dengue virus
APTRVVAAEM

SEQ ID NO: 252 - protein, 9 aa, Dengue virus
VPNYNLIIM

SEQ ID NO: 253 - protein, 10 aa, Dengue virus
APIMDEEREI

SEQ ID NO: 254 - protein, 10 aa, Dengue virus
TPEGIIPSMF

SEQ ID NO: 255 - protein, 9 aa, Dengue virus
KPRWLDARI

SEQ ID NO: 256 - protein, 10 aa, Dengue virus
RPASAWTLYA

SEQ ID NO: 257 - protein, 8 aa, Dengue virus
TPMLRHSI

SEQ ID NO: 258 - protein, 10 aa, Dengue virus
SPNPTVEAGR

SEQ ID NO: 259 - protein, 10 aa, Dengue virus
TPRMCTREEF

SEQ ID NO: 260 - protein, 9 aa, Dengue virus
RPTPRGTVM

SEQ ID NO: 261 - protein, 15 aa, Dengue virus
AFLRFLTIPPTAGIL

SEQ ID NO: 262 - protein, 15 aa, Dengue virus
GVTYLALLAAFKVRP

SEQ ID NO: 263 - protein, 15 aa, Dengue virus
MAVGMVSILASSLLK

SEQ ID NO: 264 - protein, 15 aa, Dengue virus
TFTMRLLSPVRVPNY

SEQ ID NO: 265 - protein, 15 aa, Dengue virus
SRAIWYMWLGARFLE

SEQ ID NO: 266 - protein, 9 aa, Dengue virus
ITEAELTGY

SEQ ID NO: 267 - protein, 9 aa, Dengue virus
STEIQLTDY

SEQ ID NO: 268 - protein, 9 aa, Dengue virus
TTEIQLTDY

SEQ ID NO: 269 - protein, 9 aa, Dengue virus
TSEIQLIDY

SEQ ID NO: 270 - protein, 9 aa, Dengue virus
TSEIQLTDY

SEQ ID NO: 271 - protein, 9 aa, Dengue virus
IAEAELTGY

SEQ ID NO: 272 - protein, 9 aa, Dengue virus
IAEAELTDY

SEQ ID NO: 273 - protein, 9 aa, Dengue virus
ITDAELTGY

SEQ ID NO: 274 - protein, 9 aa, Dengue virus
STEAELTGY

SEQ ID NO: 275 - protein, 9 aa, Dengue virus
TTEAELTGY

SEQ ID NO: 276 - protein, 9 aa, Dengue virus
ISEAELTDY

SEQ ID NO: 277 - protein, 9 aa, Dengue virus
ITEAELTGY

SEQ ID NO: 278 - protein, 9 aa, Dengue virus
TVEAVLLEY

SEQ ID NO: 279 - protein, 9 aa, Dengue virus
TVEAVLPEY

SEQ ID NO: 280 - protein, 9 aa, Dengue virus
TVEAILPEY

SEQ ID NO: 281 - protein, 9 aa, Dengue virus
TAEAILPEY

SEQ ID NO: 282 - protein, 9 aa, Dengue virus
THEALLPEY

SEQ ID NO: 283 - protein, 9 aa, Dengue virus
ITEAILPEY

SEQ ID NO: 284 - protein, 9 aa, Dengue virus
TTEVILPEY

SEQ ID NO: 285 - protein, 9 aa, Dengue virus
TTEAILPEY

SEQ ID NO: 286 - protein, 9 aa, Dengue virus
SVEVELPDY

SEQ ID NO: 287 - protein, 9 aa, Dengue virus
SVEVKLPDY

SEQ ID NO: 288 - protein, 10 aa, Dengue virus
RSCTLPPLRY

SEQ ID NO: 289 - protein, 10 aa, Dengue virus
RSCTLPPLRF

SEQ ID NO: 290 - protein, 10 aa, Dengue virus
RSCTLPPLRY

SEQ ID NO: 291 - protein, 10 aa, Dengue virus
RSCTLPPLRY

SEQ ID NO: 292 - protein, 10 aa, Dengue virus
RSCTMPPLRF

SEQ ID NO: 293 - protein, 9 aa, Dengue virus
MTDDIGMGV

SEQ ID NO: 294 - protein, 9 aa, Dengue virus
ASDRMGMGM

SEQ ID NO: 295 - protein, 9 aa, Dengue virus
ASDMMGMGT

SEQ ID NO: 296 - protein, 9 aa, Dengue virus
ASDKMGMGT

SEQ ID NO: 297 - protein, 9 aa, Dengue virus
ASDNMGMGT

SEQ ID NO: 298 - protein, 9 aa, Dengue virus
VSDRMGMGT

SEQ ID NO: 299 - protein, 9 aa, Dengue virus
ASDRMGMGT

SEQ ID NO: 300 - protein, 9 aa, Dengue virus
MADDIGMGV

SEQ ID NO: 301 - protein, 9 aa, Dengue virus
MTDEMGMGV

SEQ ID NO: 302 - protein, 9 aa, Dengue virus
ITDDIGMGV

SEQ ID NO: 303 - protein, 9 aa, Dengue virus
MTDDIGMGV

SEQ ID NO: 304 - protein, 9 aa, Dengue virus
ASDRTGMGV

SEQ ID NO: 305 - protein, 9 aa, Dengue virus
ASDKMGMGV

SEQ ID NO: 306 - protein, 9 aa, Dengue virus
ATDRMGMGV

SEQ ID NO: 307 - protein, 9 aa, Dengue virus
ASDRMGMGV

SEQ ID NO: 308 - protein, 9 aa, Dengue virus
LTDALALGM

SEQ ID NO: 309 - protein, 9 aa, Dengue virus
LGDGLAIGI

SEQ ID NO: 310 - protein, 9 aa, Dengue virus
LGDGFAMGI

SEQ ID NO: 311 - protein, 9 aa, Dengue virus
LGDGLAMGI

SEQ ID NO: 312 - protein, 9 aa, Dengue virus
LTDAIALGI

SEQ ID NO: 313 - protein, 9 aa, Dengue virus
LTDAWALGM

SEQ ID NO: 314 - protein, 9 aa, Dengue virus
LTDALALGI

SEQ ID NO: 315 - protein, 9 aa, Dengue virus
LTDALALGM

SEQ ID NO: 316 - protein, 9 aa, Dengue virus
MANGVALGL

SEQ ID NO: 317 - protein, 9 aa, Dengue virus
MANGIALGL

SEQ ID NO: 318 - protein, 9 aa, Dengue virus
LISGISLGL

SEQ ID NO: 319 - protein, 9 aa, Dengue virus
FIDGLSLGL

SEQ ID NO: 320 - protein, 9 aa, Dengue virus
LIDGISLGL

SEQ ID NO: 321 - protein, 9 aa, Dengue virus
LIDGIALGL

SEQ ID NO: 322 - protein, 9 aa, Dengue virus
FIDGISLGL

SEQ ID NO: 323 - protein, 9 aa, Dengue virus
VIDLDPIPY

SEQ ID NO: 324 - protein, 9 aa, Dengue virus
TIDLDPVVY

SEQ ID NO: 325 - protein, 9 aa, Dengue virus
AIDLDPVVY

SEQ ID NO: 326 - protein, 9 aa, Dengue virus
VIDLEPIPY

SEQ ID NO: 327 - protein, 9 aa, Dengue virus
VIDLDPIPY

SEQ ID NO: 328 - protein, 9 aa, Dengue virus
TIDLDSVIF

SEQ ID NO: 329 - protein, 9 aa, Dengue virus
TIDLDPVIY

SEQ ID NO: 330 - protein, 9 aa, Dengue virus
TIALDPVIY

SEQ ID NO: 331 - protein, 9 aa, Dengue virus
VIDLEPISY

SEQ ID NO: 332 - protein, 9 aa, Dengue virus
YTDYMPSMK

SEQ ID NO: 333 - protein, 9 aa, Dengue virus
YSDYMTSMK

SEQ ID NO: 334 - protein, 9 aa, Dengue virus
YLDYMASMK

SEQ ID NO: 335 - protein, 9 aa, Dengue virus
YIDYMTSMK

SEQ ID NO: 336 - protein, 9 aa, Dengue virus
YLDFMTSMK

SEQ ID NO: 337 - protein, 9 aa, Dengue virus
YLDYMTSMK

SEQ ID NO: 338 - protein, 9 aa, Dengue virus
YLDYMISMK

SEQ ID NO: 339 - protein, 9 aa, Dengue virus
YIDYMPSMK

SEQ ID NO: 340 - protein, 9 aa, Dengue virus
YMDYMPSMK

SEQ ID NO: 341 - protein, 9 aa, Dengue virus
YTDYMPSMK

SEQ ID NO: 342 - protein, 9 aa, Dengue virus
FLDYMPSMK

SEQ ID NO: 343 - protein, 9 aa, Dengue virus
YADYMPVMK

SEQ ID NO: 344 - protein, 9 aa, Dengue virus
YMDYMPVMK

SEQ ID NO: 345 - protein, 9 aa, Dengue virus
YVDYMPAMK

SEQ ID NO: 346 - protein, 9 aa, Dengue virus
YVDYMPVMR

SEQ ID NO: 347 - protein, 9 aa, Dengue virus
YVDYMPVMK

SEQ ID NO: 348 - protein, 9 aa, Dengue virus
RLITVNPIV

SEQ ID NO: 349 - protein, 9 aa, Dengue virus
RVITANPIV

SEQ ID NO: 350 - protein, 9 aa, Dengue virus
RLVTANPIV

SEQ ID NO: 351 - protein, 9 aa, Dengue virus
RLITANPIV

SEQ ID NO: 352 - protein, 9 aa, Dengue virus
RLITVNPVV

SEQ ID NO: 353 - protein, 9 aa, Dengue virus
RLITVNPII

SEQ ID NO: 354 - protein, 9 aa, Dengue virus
RLITVNPIV

SEQ ID NO: 355 - protein, 9 aa, Dengue virus
RLTTVNPIV

SEQ ID NO: 356 - protein, 9 aa, Dengue virus
RLITANPIV

SEQ ID NO: 357 - protein, 9 aa, Dengue virus
RLITANPVV

SEQ ID NO: 358 - protein, 9 aa, Dengue virus
RVISATPLA

SEQ ID NO: 359 - protein, 9 aa, Dengue virus
RVISSTPLA

SEQ ID NO: 360 - protein, 9 aa, Dengue virus
RIISSTPLA

SEQ ID NO: 361 - protein, 9 aa, Dengue virus
RVISSTPFA

SEQ ID NO: 362 - protein, 9 aa, Dengue virus
RIISSTPFA

SEQ ID NO: 363 - protein, 9 aa, Dengue virus
RIISSIPFA

SEQ ID NO: 364 - protein, 9 aa, Dengue virus
IMAVGMVSI

SEQ ID NO: 365 - protein, 9 aa, Dengue virus
IMAVGVVSI

SEQ ID NO: 366 - protein, 9 aa, Dengue virus
VMAVGIVSI

SEQ ID NO: 367 - protein, 9 aa, Dengue virus
IMAIGIVSI

SEQ ID NO: 368 - protein, 9 aa, Dengue virus
IMAVGIVSI

SEQ ID NO: 369 - protein, 9 aa, Dengue virus
VMAVGMVSI

SEQ ID NO: 370 - protein, 9 aa, Dengue virus
IMAVGMVSI

SEQ ID NO: 371 - protein, 9 aa, Dengue virus
VMAIGLVSI

SEQ ID NO: 372 - protein, 9 aa, Dengue virus
VMAVGLVSI

SEQ ID NO: 373 - protein, 9 aa, Dengue virus
MMAVGLVSL

SEQ ID NO: 374 - protein, 9 aa, Dengue virus
IMAVGLVSL

SEQ ID NO: 375 - protein, 9 aa, Dengue virus
GLLTVCYVL

SEQ ID NO: 376 - protein, 9 aa, Dengue virus
GMLITCYVI

SEQ ID NO: 377 - protein, 9 aa, Dengue virus
GMLIACYVI

SEQ ID NO: 378 - protein, 9 aa, Dengue virus
GPLTVCYVL

SEQ ID NO: 379 - protein, 9 aa, Dengue virus
GLLTVCYVL

SEQ ID NO: 380 - protein, 9 aa, Dengue virus
GMLIACYVI

SEQ ID NO: 381 - protein, 9 aa, Dengue virus
GLLIACYVI

SEQ ID NO: 382 - protein, 9 aa, Dengue virus
GLLLAAYMM

SEQ ID NO: 383 - protein, 9 aa, Dengue virus
GLLLAAYVM

SEQ ID NO: 384 - protein, 10 aa, Dengue virus
RIYSDPLALK

SEQ ID NO: 385 - protein, 10 aa, Dengue virus
RTYSDPQALR

SEQ ID NO: 386 - protein, 10 aa, Dengue virus
RTYSDPLALR

SEQ ID NO: 387 - protein, 10 aa, Dengue virus
RTYSDPLALK

SEQ ID NO: 388 - protein, 10 aa, Dengue virus
RIYSDPLTLK

SEQ ID NO: 389 - protein, 10 aa, Dengue virus
KIYSDPLALK

SEQ ID NO: 390 - protein, 10 aa, Dengue virus
RIYSEPRALK

SEQ ID NO: 391 - protein, 10 aa, Dengue virus
RIYSDPLALK

SEQ ID NO: 392 - protein, 10 aa, Dengue virus
RTYSDPLAPK

SEQ ID NO: 393 - protein, 10 aa, Dengue virus
RTYSDPLALK

SEQ ID NO: 394 - protein, 10 aa, Dengue virus
RIYSDPLALK

SEQ ID NO: 395 - protein, 10 aa, Dengue virus
RVYADPMALQ

SEQ ID NO: 396 - protein, 10 aa, Dengue virus
RVYADPMALK

SEQ ID NO: 397 - protein, 9 aa, Dengue virus
ATVLMGLGK

SEQ ID NO: 398 - protein, 9 aa, Dengue virus
AAILMGLDK

SEQ ID NO: 399 - protein, 9 aa, Dengue virus
ATVLMGLGK

SEQ ID NO: 400 - protein, 9 aa, Dengue virus
ATVLMGLGR

SEQ ID NO: 401 - protein, 9 aa, Dengue virus
AVVLMGLNK

SEQ ID NO: 402 - protein, 9 aa, Dengue virus
AVVLMGLDK

SEQ ID NO: 403 - protein, 9 aa, Dengue virus
AAVLMGLGK

SEQ ID NO: 404 - protein, 9 aa, Dengue virus
STYGWNLVR

SEQ ID NO: 405 - protein, 9 aa, Dengue virus
AAYGWNLVK

SEQ ID NO: 406 - protein, 9 aa, Dengue virus
ATYGWNLVK

SEQ ID NO: 407 - protein, 9 aa, Dengue virus
STYGWNLVR

SEQ ID NO: 408 - protein, 9 aa, Dengue virus
STYGWNLVK

SEQ ID NO: 409 - protein, 9 aa, Dengue virus
STYGWNVVK

SEQ ID NO: 410 - protein, 9 aa, Dengue virus
STYGWNIVK

SEQ ID NO: 411 - protein, 9 aa, Dengue virus
ATYGWNLVK

SEQ ID NO: 412 - protein, 9 aa, Dengue virus
TVMDIISRR

SEQ ID NO: 413 - protein, 9 aa, Dengue virus
TVMDIISRR

SEQ ID NO: 414 - protein, 9 aa, Dengue virus
TVMDVISRR

SEQ ID NO: 415 - protein, 9 aa, Dengue virus
TVLDIISRR

SEQ ID NO: 416 - protein, 9 aa, Dengue virus
TVMDIISRK

SEQ ID NO: 417 - protein, 9 aa, Dengue virus
TVMDIISRR

SEQ ID NO: 418 - protein, 9 aa, Dengue virus
TVMDIISRK

SEQ ID NO: 419 - protein, 9 aa, Dengue virus
AVMDIISRK

SEQ ID NO: 420 - protein, 10 aa, Dengue virus
RQMEGEGVFK

SEQ ID NO: 421 - protein, 10 aa, Dengue virus
RQMESEEIFS

SEQ ID NO: 422 - protein, 10 aa, Dengue virus
RQMESEGIVS

SEQ ID NO: 423 - protein, 10 aa, Dengue virus
RQMESEGIFF

SEQ ID NO: 424 - protein, 10 aa, Dengue virus
RQMESEGIIL

SEQ ID NO: 425 - protein, 10 aa, Dengue virus
RQMESEGIFS

SEQ ID NO: 426 - protein, 10 aa, Dengue virus
RQMESEGIFL

SEQ ID NO: 427 - protein, 10 aa, Dengue virus
RQMEGEGVFR

SEQ ID NO: 428 - protein, 10 aa, Dengue virus
RQMEGEGIFR

SEQ ID NO: 429 - protein, 10 aa, Dengue virus
RQMEGEGLFK

SEQ ID NO: 430 - protein, 10 aa, Dengue virus
RQMEGEEVFK

SEQ ID NO: 431 - protein, 10 aa, Dengue virus
RQMEGEGVFK

SEQ ID NO: 432 - protein, 10 aa, Dengue virus
RQMEGEGIFK

SEQ ID NO: 433 - protein, 10 aa, Dengue virus
RQMEGEGVLT

SEQ ID NO: 434 - protein, 10 aa, Dengue virus
RQMEGEGVLS

SEQ ID NO: 435 - protein, 10 aa, Dengue virus
RQMEGEDVLS

SEQ ID NO: 436 - protein, 10 aa, Dengue virus
RQMEAEGVIT

SEQ ID NO: 437 - protein, 9 aa, Dengue virus
RTTWSIHAK

SEQ ID NO: 438 - protein, 9 aa, Dengue virus
RTTWSIHAH

SEQ ID NO: 439 - protein, 9 aa, Dengue virus
RTTWSIHAR

SEQ ID NO: 440 - protein, 9 aa, Dengue virus
RTTWSIHAT

SEQ ID NO: 441 - protein, 9 aa, Dengue virus
RTTWSIHAS

SEQ ID NO: 442 - protein, 9 aa, Dengue virus
RTTWSIHAK

SEQ ID NO: 443 - protein, 9 aa, Dengue virus
RTTWSIHAH

SEQ ID NO: 444 - protein, 9 aa, Dengue virus
RTTWSIHAH

SEQ ID NO: 445 - protein, 10 aa, Dengue virus
RPTFAAGLLL

SEQ ID NO: 446 - protein, 10 aa, Dengue virus
RPMLAVGLLF

SEQ ID NO: 447 - protein, 10 aa, Dengue virus
RPMFAMGLLF

SEQ ID NO: 448 - protein, 10 aa, Dengue virus
RPMFAVGLLI

SEQ ID NO: 449 - protein, 10 aa, Dengue virus
RPMFAVGLLF

SEQ ID NO: 450 - protein, 10 aa, Dengue virus
RPTFAAGLFL

SEQ ID NO: 451 - protein, 10 aa, Dengue virus
RPTFAVGLVL

SEQ ID NO: 452 - protein, 10 aa, Dengue virus
RPTFAVGLLL

SEQ ID NO: 453 - protein, 10 aa, Dengue virus
RPTFAAGLLL

SEQ ID NO: 454 - protein, 10 aa, Dengue virus
QPFLALGFFM

SEQ ID NO: 455 - protein, 10 aa, Dengue virus
QPFLTLGFFL

SEQ ID NO: 456 - protein, 10 aa, Dengue virus
QPFLALGFFL

SEQ ID NO: 457 - protein, 10 aa, Dengue virus
SPRYVLGVFL

SEQ ID NO: 458 - protein, 10 aa, Dengue virus
SPGYVLGVFL

SEQ ID NO: 459 - protein, 10 aa, Dengue virus
SPGYVLGIFL

SEQ ID NO: 460 - protein, 9 aa, Dengue virus
LPAIVREAI

SEQ ID NO: 461 - protein, 9 aa, Dengue virus
LPAIIREAI

SEQ ID NO: 462 - protein, 9 aa, Dengue virus
LPAIVREAI

SEQ ID NO: 463 - protein, 9 aa, Dengue virus
LPAMVREAI

SEQ ID NO: 464 - protein, 9 aa, Dengue virus
LPAIVREAI

SEQ ID NO: 465 - protein, 9 aa, Dengue virus
LPTIVREAI

SEQ ID NO: 466 - protein, 9 aa, Dengue virus
LPAVVREAI

SEQ ID NO: 467 - protein, 9 aa, Dengue virus
LPAIVREAI

SEQ ID NO: 468 - protein, 9 aa, Dengue virus
LPAIIREAI

SEQ ID NO: 469 - protein, 9 aa, Dengue virus
LPSIVREAL

SEQ ID NO: 470 - protein, 10 aa, Dengue virus
APTRVVAAEM

SEQ ID NO: 471 - protein, 10 aa, Dengue virus
APTRVVASET

SEQ ID NO: 472 - protein, 10 aa, Dengue virus
APTRVVAAEM

SEQ ID NO: 473 - protein, 10 aa, Dengue virus
APTRVVASEM

SEQ ID NO: 474 - protein, 10 aa, Dengue virus
APPRVVPAEM

SEQ ID NO: 475 - protein, 10 aa, Dengue virus
APTRVVAAEM

SEQ ID NO: 476 - protein, 10 aa, Dengue virus
APTRVVAAEM

SEQ ID NO: 477 - protein, 10 aa, Dengue virus
APTRVVAAEM

SEQ ID NO: 478 - protein, 9 aa, Dengue virus
VPNYNLIIM

SEQ ID NO: 479 - protein, 9 aa, Dengue virus
VPNYNMIIV

SEQ ID NO: 480 - protein, 9 aa, Dengue virus
VPNYNMIIM

SEQ ID NO: 481 - protein, 9 aa, Dengue virus
VPNYNMIVM

SEQ ID NO: 482 - protein, 9 aa, Dengue virus
VPNYNLIIM

SEQ ID NO: 483 - protein, 9 aa, Dengue virus
VPNYNLIVM

SEQ ID NO: 484 - protein, 9 aa, Dengue virus
VPNYNLVVM

SEQ ID NO: 485 - protein, 9 aa, Dengue virus
VPNYNLVIM

SEQ ID NO: 486 - protein, 9 aa, Dengue virus
VSNYNLIIM

SEQ ID NO: 487 - protein, 9 aa, Dengue virus
VPNYNLIIM

SEQ ID NO: 488 - protein, 9 aa, Dengue virus
VPNYNLIVM

SEQ ID NO: 489 - protein, 10 aa, Dengue virus
APIMDEEREI

SEQ ID NO: 490 - protein, 10 aa, Dengue virus
AIIQDEERDI

SEQ ID NO: 491 - protein, 10 aa, Dengue virus
AVIQDEEKDI

SEQ ID NO: 492 - protein, 10 aa, Dengue virus
AAIQDEERDI

SEQ ID NO: 493 - protein, 10 aa, Dengue virus
AVIQDEERDI

SEQ ID NO: 494 - protein, 10 aa, Dengue virus
APIMDDEREI

SEQ ID NO: 495 - protein, 10 aa, Dengue virus
APIIDEEREI

SEQ ID NO: 496 - protein, 10 aa, Dengue virus
APIVDEEREI

SEQ ID NO: 497 - protein, 10 aa, Dengue virus
APIMDEEREI

SEQ ID NO: 498 - protein, 10 aa, Dengue virus
APIQDEEKDI

SEQ ID NO: 499 - protein, 10 aa, Dengue virus
SPIQDEERDI

SEQ ID NO: 500 - protein, 10 aa, Dengue virus
APIQDEERDI

SEQ ID NO: 501 - protein, 10 aa, Dengue virus
APIQDKERDI

SEQ ID NO: 502 - protein, 10 aa, Dengue virus
SPIEDIEREI

SEQ ID NO: 503 - protein, 10 aa, Dengue virus
TPEGIIPSMF

SEQ ID NO: 504 - protein, 10 aa, Dengue virus
TPEGIIPALY

SEQ ID NO: 505 - protein, 10 aa, Dengue virus
TPEGIIPALF

SEQ ID NO: 506 - protein, 10 aa, Dengue virus
TPEGIIPSLF

SEQ ID NO: 507 - protein, 10 aa, Dengue virus
TPEGIIPSMF

SEQ ID NO: 508 - protein, 10 aa, Dengue virus
TPEGIIPALF

SEQ ID NO: 509 - protein, 10 aa, Dengue virus
TPEGIIPTLF

SEQ ID NO: 510 - protein, 10 aa, Dengue virus
LLVISGLFPV

SEQ ID NO: 511 - protein, 10 aa, Dengue virus
LLAISGVYPL

SEQ ID NO: 512 - protein, 10 aa, Dengue virus
LLAVSGMYPL

SEQ ID NO: 513 - protein, 10 aa, Dengue virus
LLAVSGVYPL

SEQ ID NO: 514 - protein, 10 aa, Dengue virus
LLVISGVYPM

SEQ ID NO: 515 - protein, 10 aa, Dengue virus
LLAVSGVYPI

SEQ ID NO: 516 - protein, 10 aa, Dengue virus
LLAASGVYPM

SEQ ID NO: 517 - protein, 10 aa, Dengue virus
LLAISGVYPM

SEQ ID NO: 518 - protein, 10 aa, Dengue virus
LLAVSGVYPM

SEQ ID NO: 519 - protein, 10 aa, Dengue virus
LLVVSGLFPV

SEQ ID NO: 520 - protein, 10 aa, Dengue virus
LLVISGLFPA

SEQ ID NO: 521 - protein, 10 aa, Dengue virus
LLVISGLFPI

SEQ ID NO: 522 - protein, 10 aa, Dengue virus
LLVISGVFPV

SEQ ID NO: 523 - protein, 10 aa, Dengue virus
LLVISGLFPV

SEQ ID NO: 524 - protein, 10 aa, Dengue virus
LLIVSGIFPC

SEQ ID NO: 525 - protein, 10 aa, Dengue virus
LLIVSGIFPY

SEQ ID NO: 526 - protein, 10 aa, Dengue virus
LLIVSGVFPY

SEQ ID NO: 527 - protein, 10 aa, Dengue virus
LITVSGLYPL

SEQ ID NO: 528 - protein, 9 aa, Dengue virus
AAAWYLWEV

SEQ ID NO: 529 - protein, 9 aa, Dengue virus
LFVWCFWQK

SEQ ID NO: 530 - protein, 9 aa, Dengue virus
LFLWYFWQK

SEQ ID NO: 531 - protein, 9 aa, Dengue virus
LFVWHFWQK

SEQ ID NO: 532 - protein, 9 aa, Dengue virus
FFVWYFWQK

SEQ ID NO: 533 - protein, 9 aa, Dengue virus
PFVWYFWQK

SEQ ID NO: 534 - protein, 9 aa, Dengue virus
LFVWYFWQK

SEQ ID NO: 535 - protein, 9 aa, Dengue virus
AAAWYLWET

SEQ ID NO: 536 - protein, 9 aa, Dengue virus
AAAWYLWEA

SEQ ID NO: 537 - protein, 9 aa, Dengue virus
AAAWYLWEV

SEQ ID NO: 538 - protein, 9 aa, Dengue virus
LLVWHAWQK

SEQ ID NO: 539 - protein, 9 aa, Dengue virus
MLVWHTWQK

SEQ ID NO: 540 - protein, 9 aa, Dengue virus
LLVWHTWQK

SEQ ID NO: 541 - protein, 9 aa, Dengue virus
MALWYIWQV

SEQ ID NO: 542 - protein, 9 aa, Dengue virus
MTLWYMWQV

SEQ ID NO: 543 - protein, 9 aa, Dengue virus
MALWYMWQV

SEQ ID NO: 544 - protein, 9 aa, Dengue virus
YLPAIVREA

SEQ ID NO: 545 - protein, 9 aa, Dengue virus
YLPAIIREA

SEQ ID NO: 546 - protein, 9 aa, Dengue virus
YLPAIVREA

SEQ ID NO: 547 - protein, 9 aa, Dengue virus
YLPAMVREA

SEQ ID NO: 548 - protein, 9 aa, Dengue virus
SLPAIVREA

SEQ ID NO: 549 - protein, 9 aa, Dengue virus
YLPAIVREA

SEQ ID NO: 550 - protein, 9 aa, Dengue virus
YLPTIVREA

SEQ ID NO: 551 - protein, 9 aa, Dengue virus
YLPAVVREA

SEQ ID NO: 552 - protein, 9 aa, Dengue virus
YLPAIVREA

SEQ ID NO: 553 - protein, 9 aa, Dengue virus
YLPAIIREA

SEQ ID NO: 554 - protein, 9 aa, Dengue virus
ILPSIVREA

SEQ ID NO: 555 - protein, 10 aa, Dengue virus
DLMRRGDLPV

SEQ ID NO: 556 - protein, 10 aa, Dengue virus
DLLRRGDLPV

SEQ ID NO: 557 - protein, 10 aa, Dengue virus
ELMRRGDLPV

SEQ ID NO: 558 - protein, 10 aa, Dengue virus
DLMKRGDLPV

SEQ ID NO: 559 - protein, 10 aa, Dengue virus
ELMRRGDLPV

SEQ ID NO: 560 - protein, 10 aa, Dengue virus
DLMRRGDLPV

SEQ ID NO: 561 - protein, 10 aa, Dengue virus
ELMRRGHLPV

SEQ ID NO: 562 - protein, 10 aa, Dengue virus
ELMRRGDLPV

SEQ ID NO: 563 - protein, 10 aa, Dengue virus
ELMKRGDLPV

SEQ ID NO: 564 - protein, 10 aa, Dengue virus
ELMRRGDLPV

SEQ ID NO: 565 - protein, 9 aa, Dengue virus
ALSELPETL

SEQ ID NO: 566 - protein, 9 aa, Dengue virus
ALEELPDTI

SEQ ID NO: 567 - protein, 9 aa, Dengue virus
AVEELPDTI

SEQ ID NO: 568 - protein, 9 aa, Dengue virus
AMEELPDTI

SEQ ID NO: 569 - protein, 9 aa, Dengue virus
ALSELAETL

SEQ ID NO: 570 - protein, 9 aa, Dengue virus
ALGELPETL

SEQ ID NO: 571 - protein, 9 aa, Dengue virus
ALSELPETL

SEQ ID NO: 572 - protein, 9 aa, Dengue virus
AVEELPETM

SEQ ID NO: 573 - protein, 9 aa, Dengue virus
ALNELTESL

SEQ ID NO: 574 - protein, 9 aa, Dengue virus
ALNELPESL

SEQ ID NO: 575 - protein, 9 aa, Dengue virus
IILEFFLIV

SEQ ID NO: 576 - protein, 9 aa, Dengue virus
IILKFFLMV

SEQ ID NO: 577 - protein, 9 aa, Dengue virus
IILEFLLMV

SEQ ID NO: 578 - protein, 9 aa, Dengue virus
IMLEFFLMV

SEQ ID NO: 579 - protein, 9 aa, Dengue virus
IILEFFLMV

SEQ ID NO: 580 - protein, 9 aa, Dengue virus
IILEFFLMV

SEQ ID NO: 581 - protein, 9 aa, Dengue virus
IILEFFLIV

SEQ ID NO: 582 - protein, 9 aa, Dengue virus
IILEFFMMV

SEQ ID NO: 583 - protein, 9 aa, Dengue virus
IVLEFFMMV

SEQ ID NO: 584 - protein, 9 aa, Dengue virus
IILEFFLMV

SEQ ID NO: 585 - protein, 9 aa, Dengue virus
KLAEAIFKL

SEQ ID NO: 586 - protein, 9 aa, Dengue virus
LLAKAIFKL

SEQ ID NO: 587 - protein, 9 aa, Dengue virus
QLAKSIFKL

SEQ ID NO: 588 - protein, 9 aa, Dengue virus
LLATSVFKL

SEQ ID NO: 589 - protein, 9 aa, Dengue virus
LLAKSIFKL

SEQ ID NO: 590 - protein, 9 aa, Dengue virus
LLATAIFKL

SEQ ID NO: 591 - protein, 9 aa, Dengue virus
LLATSIFKL

SEQ ID NO: 592 - protein, 9 aa, Dengue virus
LLASSIFKL

SEQ ID NO: 593 - protein, 9 aa, Dengue virus
KLAEAIFRL

SEQ ID NO: 594 - protein, 9 aa, Dengue virus
RLAEAIFKL

SEQ ID NO: 595 - protein, 9 aa, Dengue virus
KLAEAVFKL

SEQ ID NO: 596 - protein, 9 aa, Dengue virus
KLAEAIFKL

SEQ ID NO: 597 - protein, 9 aa, Dengue virus
QLASAIFKL

SEQ ID NO: 598 - protein, 9 aa, Dengue virus
LLANAIFKL

SEQ ID NO: 599 - protein, 9 aa, Dengue virus
RLANAIFKL

SEQ ID NO: 600 - protein, 9 aa, Dengue virus
QLANAIFKL

SEQ ID NO: 601 - protein, 9 aa, Dengue virus
TLAKAIFKL

SEQ ID NO: 602 - protein, 9 aa, Dengue virus
ILAKAIFKL

SEQ ID NO: 603 - protein, 9 aa, Dengue virus
SQIGAGVYK

SEQ ID NO: 604 - protein, 9 aa, Dengue virus
SQVGVGVFQ

SEQ ID NO: 605 - protein, 9 aa, Dengue virus
SQIGAGVYR

SEQ ID NO: 606 - protein, 9 aa, Dengue virus
SQIGTGVYK

SEQ ID NO: 607 - protein, 9 aa, Dengue virus
SQIGVGVYK

SEQ ID NO: 608 - protein, 9 aa, Dengue virus
SQIGAGVYK

SEQ ID NO: 609 - protein, 9 aa, Dengue virus
TQVGVGIQK

SEQ ID NO: 610 - protein, 9 aa, Dengue virus
TQVGVGVHK

SEQ ID NO: 611 - protein, 9 aa, Dengue virus
TQVGVGVQK

SEQ ID NO: 612 - protein, 9 aa, Dengue virus
TQVGVGIHI

SEQ ID NO: 613 - protein, 9 aa, Dengue virus
TQVGVGIHT

SEQ ID NO: 614 - protein, 9 aa, Dengue virus
TQVGVGIHM

SEQ ID NO: 615 - protein, 9 aa, Dengue virus
TQVGVGVHV

SEQ ID NO: 616 - protein, 10 aa, Dengue virus
GTSGSPIIDK

SEQ ID NO: 617 - protein, 10 aa, Dengue virus
GTSGSPIVSR

SEQ ID NO: 618 - protein, 10 aa, Dengue virus
GTSGSPIVNR

SEQ ID NO: 619 - protein, 10 aa, Dengue virus
GTSGSPIIDK

SEQ ID NO: 620 - protein, 10 aa, Dengue virus
GTSGSPIADK

SEQ ID NO: 621 - protein, 10 aa, Dengue virus
GTSGSPIVDR

SEQ ID NO: 622 - protein, 10 aa, Dengue virus
GTSGSPIVDK

SEQ ID NO: 623 - protein, 10 aa, Dengue virus
GTSGSPIINK

SEQ ID NO: 624 - protein, 10 aa, Dengue virus
GTSGSPIINR

SEQ ID NO: 625 - protein, 10 aa, Dengue virus
GSSGSPIINR

SEQ ID NO: 626 - protein, 10 aa, Dengue virus
GTSGSPIVNR

SEQ ID NO: 627 - protein, 10 aa, Dengue virus
GTSGSPIINK

SEQ ID NO: 628 - protein, 10 aa, Dengue virus
GTSGSPIINR

SEQ ID NO: 629 - protein, 9 aa, Dengue virus
KTFDSEYVK

SEQ ID NO: 630 - protein, 9 aa, Dengue virus
KTFDTEYQK

SEQ ID NO: 631 - protein, 9 aa, Dengue virus
KTFDTEYTK

SEQ ID NO: 632 - protein, 9 aa, Dengue virus
KTFDTEYIK

SEQ ID NO: 633 - protein, 9 aa, Dengue virus
KTFDFEYIK

SEQ ID NO: 634 - protein, 9 aa, Dengue virus
KTFDSEYIK

SEQ ID NO: 635 - protein, 9 aa, Dengue virus
KTFDSEYAK

SEQ ID NO: 636 - protein, 9 aa, Dengue virus
KTFDSEYVK

SEQ ID NO: 637 - protein, 9 aa, Dengue virus
KTFDTEYQR

SEQ ID NO: 638 - protein, 9 aa, Dengue virus
KTFNTEYQK

SEQ ID NO: 639 - protein, 9 aa, Dengue virus
KTFDTEYQK

SEQ ID NO: 640 - protein, 9 aa, Dengue virus
KTFDTEYPK

SEQ ID NO: 641 - protein, 9 aa, Dengue virus
KPRWLDARI

SEQ ID NO: 642 - protein, 9 aa, Dengue virus
RPRWLDART

SEQ ID NO: 643 - protein, 9 aa, Dengue virus
KPRWLDART

SEQ ID NO: 644 - protein, 9 aa, Dengue virus
KPRWLDAKI

SEQ ID NO: 645 - protein, 9 aa, Dengue virus
KPRWLDPRI

SEQ ID NO: 646 - protein, 9 aa, Dengue virus
KPRWLDARI

SEQ ID NO: 647 - protein, 9 aa, Dengue virus
RPRWLDART

SEQ ID NO: 648 - protein, 9 aa, Dengue virus
RPRWLDARI

SEQ ID NO: 649 - protein, 9 aa, Dengue virus
RPRWLDARV

SEQ ID NO: 650 - protein, 9 aa, Dengue virus
RPKWLDARV

SEQ ID NO: 651 - protein, 10 aa, Dengue virus
RPASAWTLYA

SEQ ID NO: 652 - protein, 10 aa, Dengue virus
HPASAWTLYA

SEQ ID NO: 653 - protein, 10 aa, Dengue virus
RPASAWTLYA

SEQ ID NO: 654 - protein, 10 aa, Dengue virus
RPASAWTLYA

SEQ ID NO: 655 - protein, 10 aa, Dengue virus
HPASAWILYA

SEQ ID NO: 656 - protein, 10 aa, Dengue virus
HPASAWTLYA

SEQ ID NO: 657 - protein, 10 aa, Dengue virus
RPASAWTLYA

SEQ ID NO: 658 - protein, 8 aa, Dengue virus
TPMLRHSI

SEQ ID NO: 659 - protein, 8 aa, Dengue virus
TPMLRHTI

SEQ ID NO: 660 - protein, 8 aa, Dengue virus
TPMMRHTI

SEQ ID NO: 661 - protein, 8 aa, Dengue virus
TPMLRHSI

SEQ ID NO: 662 - protein, 8 aa, Dengue virus
TPMLRHTI

SEQ ID NO: 663 - protein, 8 aa, Dengue virus
TPMLRHTI

SEQ ID NO: 664 - protein, 10 aa, Dengue virus
SPNPTVEAGR

SEQ ID NO: 665 - protein, 10 aa, Dengue virus
SPNPTIEEGR

SEQ ID NO: 666 - protein, 10 aa, Dengue virus
SPSPTVEAGR

SEQ ID NO: 667 - protein, 10 aa, Dengue virus
SPNPTVDAGR

SEQ ID NO: 668 - protein, 10 aa, Dengue virus
SPNPTVEAGP

SEQ ID NO: 669 - protein, 10 aa, Dengue virus
SPNPTIEAGR

SEQ ID NO: 670 - protein, 10 aa, Dengue virus
SPNPTVEAGR

SEQ ID NO: 671 - protein, 10 aa, Dengue virus
SPSPTVEEGR

SEQ ID NO: 672 - protein, 10 aa, Dengue virus
SPSLTVEESR

SEQ ID NO: 673 - protein, 10 aa, Dengue virus
SPSPIVEESR

SEQ ID NO: 674 - protein, 10 aa, Dengue virus
SPSPTVEESR

SEQ ID NO: 675 - protein, 10 aa, Dengue virus
SSNPTIEEGR

SEQ ID NO: 676 - protein, 10 aa, Dengue virus
TPRMCTREEF

SEQ ID NO: 677 - protein, 10 aa, Dengue virus
KPRICTREEF

SEQ ID NO: 678 - protein, 10 aa, Dengue virus
RPRICTRAEF

SEQ ID NO: 679 - protein, 10 aa, Dengue virus
KPRICTRAEF

SEQ ID NO: 680 - protein, 10 aa, Dengue virus
TRRMCTREEF

SEQ ID NO: 681 - protein, 10 aa, Dengue virus
TPRICTREEF

SEQ ID NO: 682 - protein, 10 aa, Dengue virus
IPRMCTREEF

SEQ ID NO: 683 - protein, 10 aa, Dengue virus
TPRMCTREEF

SEQ ID NO: 684 - protein, 10 aa, Dengue virus
KPRLCPREEF

SEQ ID NO: 685 - protein, 10 aa, Dengue virus
KPRLCTREEF

SEQ ID NO: 686 - protein, 10 aa, Dengue virus
RPRLCTREEF

SEQ ID NO: 687 - protein, 10 aa, Dengue virus
NPRLCTKEEF

SEQ ID NO: 688 - protein, 10 aa, Dengue virus
SPRLCTREEF

SEQ ID NO: 689 - protein, 10 aa, Dengue virus
TPRLCTREEF

SEQ ID NO: 690 - protein, 10 aa, Dengue virus
SPRLCTKEEF

SEQ ID NO: 691 - protein, 10 aa, Dengue virus
NPRLCTREEF

SEQ ID NO: 692 - protein, 10 aa, Dengue virus
KPRLCTREEF

SEQ ID NO: 693 - protein, 9 aa, Dengue virus
RPTPRGTVM

SEQ ID NO: 694 - protein, 9 aa, Dengue virus
RPVKNGTVM

SEQ ID NO: 695 - protein, 9 aa, Dengue virus
RPARNGTVM

SEQ ID NO: 696 - protein, 9 aa, Dengue virus
RPAKNGTVM

SEQ ID NO: 697 - protein, 9 aa, Dengue virus
RPAKSGTVM

SEQ ID NO: 698 - protein, 9 aa, Dengue virus
RPTPRGTVL

SEQ ID NO: 699 - protein, 9 aa, Dengue virus
RPTPKGTVM

SEQ ID NO: 700 - protein, 9 aa, Dengue virus
RPTPIGTVM

SEQ ID NO: 701 - protein, 9 aa, Dengue virus
RPTPRGTVM

SEQ ID NO: 702 - protein, 9 aa, Dengue virus
RPTPKGTVM

SEQ ID NO: 703 - protein, 9 aa, Dengue virus
RPTPTGTVM

SEQ ID NO: 704 - protein, 9 aa, Dengue virus
RPTPRGAVM

SEQ ID NO: 705 - protein, 9 aa, Dengue virus
RPTPKGAVM

SEQ ID NO: 706 - protein, 15 aa, Dengue virus
AFLRFLTIPPTAGIL

SEQ ID NO: 707 - protein, 15 aa, Dengue virus
AFLRFLAIPPTAGIV

SEQ ID NO: 708 - protein, 15 aa, Dengue virus
ALLRFLAIPPTAGIL

SEQ ID NO: 709 - protein, 15 aa, Dengue virus
AFLTFLAIPPTAGIL

SEQ ID NO: 710 - protein, 15 aa, Dengue virus
AFLRFLAIPPTAGIL

SEQ ID NO: 711 - protein, 15 aa, Dengue virus
AFLRFLTISPTAGIL

SEQ ID NO: 712 - protein, 15 aa, Dengue virus
AFLRFLTIPPTVGIL

SEQ ID NO: 713 - protein, 15 aa, Dengue virus
AFLRFLTIPPTAGIL

SEQ ID NO: 714 - protein, 15 aa, Dengue virus
AFLRFLAIPPTAGIL

SEQ ID NO: 715 - protein, 15 aa, Dengue virus
AFLRFLAIPPTAGVL

SEQ ID NO: 716 - protein, 15 aa, Dengue virus
TFLRVLSIPPTAGIL

SEQ ID NO: 717 - protein, 15 aa, Dengue virus
GVTYLALLAAFKVRP

SEQ ID NO: 718 - protein, 15 aa, Dengue virus
GTTYLALMATFRMRP

SEQ ID NO: 719 - protein, 15 aa, Dengue virus
GMTYLALMATFKMRP

SEQ ID NO: 720 - protein, 15 aa, Dengue virus
GTTYLALMATLKMRP

SEQ ID NO: 721 - protein, 15 aa, Dengue virus
GTTHLALMATFKMRP

SEQ ID NO: 722 - protein, 15 aa, Dengue virus
GTTYLALMATFKMRP

SEQ ID NO: 723 - protein, 15 aa, Dengue virus
GVTYLALLATFKVRP

SEQ ID NO: 724 - protein, 15 aa, Dengue virus
GVTYLALLAAYKVRP

SEQ ID NO: 725 - protein, 15 aa, Dengue virus
GVTYLALLAAFRVRP

SEQ ID NO: 726 - protein, 15 aa, Dengue virus
GVTYLALLAAFKVRP

SEQ ID NO: 727 - protein, 15 aa, Dengue virus
GVTYLALIATFEIQP

SEQ ID NO: 728 - protein, 15 aa, Dengue virus
GVTCLALIATFKIQP

SEQ ID NO: 729 - protein, 15 aa, Dengue virus
GVTYLALIATFKVQP

SEQ ID NO: 730 - protein, 15 aa, Dengue virus
GVTYLALIATFKIQP

SEQ ID NO: 731 - protein, 15 aa, Dengue virus
GQTHLAIMAVFKMSP

SEQ ID NO: 732 - protein, 15 aa, Dengue virus
GQIHLAIMAVFKMSP

SEQ ID NO: 733 - protein, 15 aa, Dengue virus
GQTHLAIMIVFKMSP

SEQ ID NO: 734 - protein, 15 aa, Dengue virus
GQVHLAIMAVFKMSP

SEQ ID NO: 735 - protein, 15 aa, Dengue virus
GQIHLAIMTMFKMSP

SEQ ID NO: 736 - protein, 15 aa, Dengue virus
MAVGMVSILASSLLK

SEQ ID NO: 737 - protein, 15 aa, Dengue virus
MAVGVVSILLSSLLK

SEQ ID NO: 738 - protein, 15 aa, Dengue virus
MAIGIVSILLSSLLK

SEQ ID NO: 739 - protein, 15 aa, Dengue virus
MAVGIVSILLSSLLK

SEQ ID NO: 740 - protein, 15 aa, Dengue virus
MAVGMVSILASSLLK

SEQ ID NO: 741 - protein, 15 aa, Dengue virus
MAVGLVSILASSFLR

SEQ ID NO: 742 - protein, 15 aa, Dengue virus
MAIGLVSILASSLLR

SEQ ID NO: 743 - protein, 15 aa, Dengue virus
MAVGLVSILASSLLR

SEQ ID NO: 744 - protein, 15 aa, Dengue virus
MAVGLVSLLGSALLK

SEQ ID NO: 745 - protein, 15 aa, Dengue virus
TFTMRLLSPVRVPNY

SEQ ID NO: 746 - protein, 15 aa, Dengue virus
TFTMRLLSPVRVPNY

SEQ ID NO: 747 - protein, 15 aa, Dengue virus
PFTMRLLSPVRVPNY

SEQ ID NO: 748 - protein, 15 aa, Dengue virus
TFTMRLLSPIRVPNY

SEQ ID NO: 749 - protein, 15 aa, Dengue virus
TFTMRLLSPVRVPNY

SEQ ID NO: 750 - protein, 15 aa, Dengue virus
TFTMRLLSPVRVSNY

SEQ ID NO: 751 - protein, 15 aa, Dengue virus
PFTMRLLSPVRVPNY

SEQ ID NO: 752 - protein, 15 aa, Dengue virus
TFTMRLLSPVRVPNY

SEQ ID NO: 753 - protein, 15 aa, Dengue virus
TFTTKLLSSTRVPNY

SEQ ID NO: 754 - protein, 15 aa, Dengue virus
TFTTRLLSSTRVPNY

SEQ ID NO: 755 - protein, 15 aa, Dengue virus
SRAIWYMWLGARFLE

SEQ ID NO: 756 - protein, 15 aa, Dengue virus
SRAIWYVWLGARFLE

SEQ ID NO: 757 - protein, 15 aa, Dengue virus
SRAIWYMWLGAAFLE

SEQ ID NO: 758 - protein, 15 aa, Dengue virus
SRAIWYMWLGARFLE

SEQ ID NO: 759 - protein, 15 aa, Dengue virus
SRAIWYMWLGARFLE

SEQ ID NO: 760 - protein, 15 aa, Dengue virus
SRAIWYMWLGARFLE

SEQ ID NO: 761 - protein, 15 aa, Dengue virus
SRAIWYMWLGVRYLE

SEQ ID NO: 762 - protein, 15 aa, Dengue virus
SRAIWYMWLGARYLE

SEQ ID NO: 763 - protein, 15 aa, Dengue virus
SRAIWYMWLGARFLE